(19) United States
(12) Patent Application Publication
(10) Pub. No.: US 2022/0281848 A1
(43) Pub. Date: Sep. 8, 2022

(54) PYRAZOLE-SUBSTITUTED PYRROLIDINONES AS HERBICIDES

(71) Applicant: FMC Corporation, Philadelphia, PA (US)

(72) Inventors: James Alan Morris, Berkshire (GB); Travis Chandler McMahon, Middletown, DE (US); Thomas Martin Stevenson, Newark, DE (US); Matthew James Campbell, Flemington, NJ (US); Sean Ng, Berkshire (GB); Sally Elizabeth Russell, Berkshire (GB)

(21) Appl. No.: 17/614,073

(22) PCT Filed: May 22, 2020
(86) PCT No.: PCT/US20/34232

§ 371 Date: Nov. 24, 2021

Related U.S. Application Data
(60) Provisional application No. 62/852,971, filed on May 24, 2019.

(51) Int. Cl. C07D 403/04; A01N 43/56; A01P 13/00

(52) U.S. Cl. CPC C07D 403/04; A01N 43/56; A01P 13/00

(57) ABSTRACT

Disclosed are compounds of Formula 1, including all stereoisomers, N-oxides, and salts thereof,

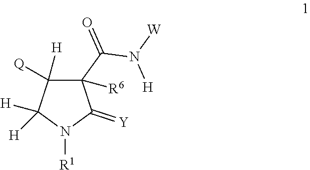

wherein Q is selected from the group consisting of

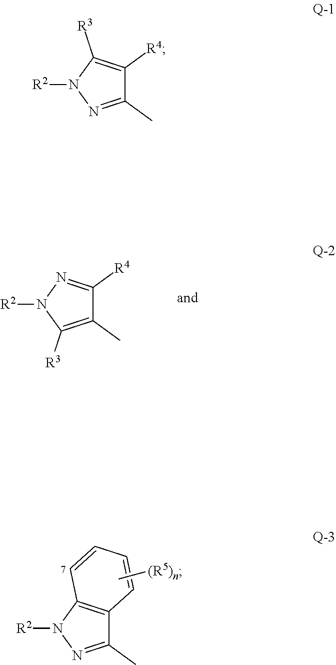

and R¹, R², R³, Y, R⁴, R⁵, n, R⁶, W and R⁹ are disclosed herein.
Also disclosed are compositions containing the compounds of Formula 1 and methods for controlling undesired vegetation comprising contacting the undesired vegetation or its environment with an effective amount of a compound or a composition of the invention.

### FIELD OF THE INVENTION

This invention relates to certain pyrazole-substituted pyrrolidinones, their N-oxides, salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action.

WO 2015/084796 and WO 2016/196593 disclose certain substituted cyclic amides and their use as herbicides. The pyrazole-substituted pyrrolidinones of the present invention are not disclosed in these publications.

### SUMMARY OF THE INVENTION

This invention is directed to a compound of Formula 1 including all stereoisomers, N-oxides, and salts thereof, agricultural compositions containing them and their use as herbicides:

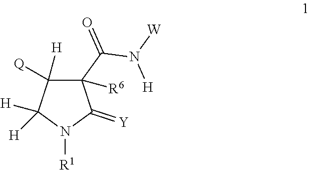

wherein Q is selected from the group consisting of

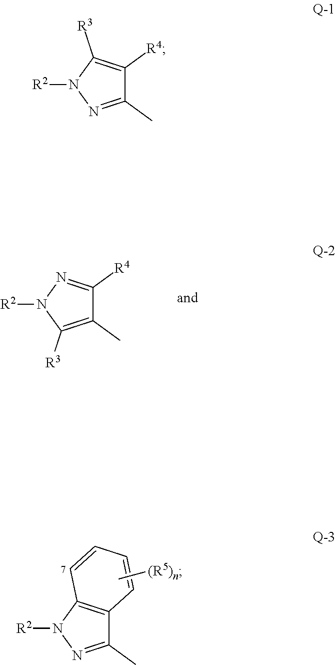

R¹ is H, C₁-C₆ alkyl, C₁-C₆ haloalkyl, C₃-C₇ cycloalkyl or C₄-C₈ cycloalkylalkyl;
R² is C₁-C₆ alkyl or C₁-C₆ haloalkyl;
R³ is halogen, C₁-C₆ alkyl, C₁-C₆ haloalkyl, C₁-C₆ alkoxy or C₁—C haloalkoxy;
Y is O or S;
R⁴ is H, halogen, C₁-C₄ alkyl or C₁-C₄ haloalkyl;
R⁵ is halogen, C₁-C₄ alkyl or C₁-C₄ haloalkyl;
n is 1, 2, 3 or 4;
R⁶ is H, halogen, hydroxy, C₁-C₄ alkoxy, C₁-C₄ haloalkyl or C₁-C₄ alkyl;
W is phenyl or pyridyl, each phenyl or pyridyl optionally substituted with up to 5 R⁹; and
each R⁹ is independently halogen, cyano, nitro, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ nitroalkyl, C₂-C₄ nitroalkenyl, C₂-C₄ alkoxyalkyl, C₂-C₄ haloalkoxyalkyl, C₃-C₄ cycloalkyl, C₃-C₄ halocycloalkyl, cyclopropylmethyl, methylcyclopropyl, C₁-C₄ alkoxy, C₁-C₄ haloalkoxy, C₂-C₄ alkenyloxy, C₂-C₄ haloalkenyloxy, C₃-C₄ alkynyloxy, C₃-C₄ haloalkynyloxy, C₃-C₄ cycloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, hydroxy, formyl, C₂-C₄ alkylcarbonyl, C₂-C₄ alkylcarbonyloxy, C₁-C₄ alkylsulfonyloxy, C₁-C₄ haloalkylsulfonyloxy, amino, C₁-C₄ alkylamino, C₂-C₄ dialkylamino, formylamino, C₂-C₄ alkylcarbonylamino, —SF₅, —SCN, C₃-C₄ trialkylsilyl, trimethylsilylmethyl or trimethylsilylmethoxy;
provided the compound is other than a compound of Formula 1 wherein Q is Q-1; R¹ is H; R² is CH₃; R³ is C(CH₃)₃; R⁴ is H; R⁶ is H; Y is O, W is phenyl substituted with R⁹ at the 2-position; and R⁹ is F.

More particularly, this invention pertains to a compound of Formula 1 (including all stereoisomers), an N-oxide or a salt thereof. This invention also relates to a herbicidal composition comprising a compound of the invention (i.e. in a herbicidally effective amount) and at least one component selected from the group consisting of surfactants, solid diluents and liquid diluents. This invention further relates to a method for controlling the growth of undesired vegetation comprising contacting the vegetation or its environment with a herbicidally effective amount of a compound of the invention (e.g., as a composition described herein).

This invention also includes a herbicidal mixture comprising (a) a compound selected from Formula 1, N-oxides, and salts thereof, and (b) at least one additional active ingredient selected from (b1) through (b16); and salts of compounds of (b1) through (b16), as described below.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention.

The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals specified for R³ and R⁴.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,3-butadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkenyloxy” includes straight-chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC═CCH₂O, CH₃C═CCH₂O and CH₃C═CCH₂CH₂O. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylamino”, “dialkylamino”, and the like, are defined analogously to the above examples.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkoxy” denotes cycloalkyl linked through an oxygen atom such as cyclopentyloxy and cyclohexyloxy. “Cycloalkylalkoxy” denotes cycloalkylalkyl linked through an oxygen atom attached to the alkyl chain. Examples of “cycloalkylalkoxy” include cyclopropylmethoxy, cyclopentylethoxy, and other cycloalkyl moieties bonded to straight-chain or branched alkoxy groups.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “halocycloalkyl”, “haloalkoxy”, “haloalkylthio”, “haloalkylsulfinyl”, “haloalkylsulfonyl”, “haloalkenyl”, “haloalkynyl”, and the like, are defined analogously to the term “haloalkyl”. Examples of “halocycloalkyl” include 2-chlorocyclopropyl and 2-bromocycbutyl. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HC═CCHCl—, CF₃C═C—, CCl₃C═C— and FCH₂C═CCH₂—.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents, e.g., ([R⁽⁵⁾)ₙ], n is 1, 2, 3 or 4). When a group contains a substituent which can be hydrogen, for example (R¹ or R⁴), then when this substituent is taken as hydrogen, it is recognized that this is equivalent to said group being unsubstituted. The term “optionally substituted” in connection with the phenyl or pyridyl, this refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

As noted above, W can be phenyl or pyridyl, each phenyl or pyridyl optionally substituted with up to 5 R⁹. Examples of phenyl or pyridyl include the following:

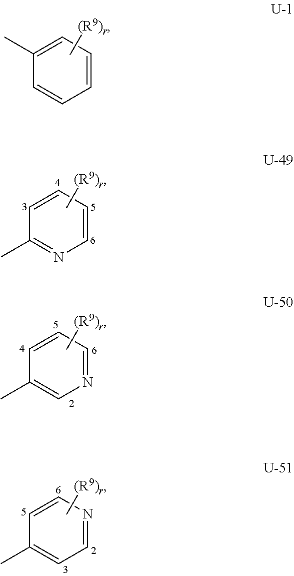

Although R⁹ groups are shown in the structures U-1, U-49, U-50 and U-51, it is noted that they do not need to be present since they are optional substituents. Note that when the attachment point between (R⁹)ᵣ and the U group is illustrated as floating, (R⁹)ᵣ can be attached to any available carbon atom of the U group.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

For example, as shown in the following, the C(O)NH(W) moiety bonded to the carbon at the 3-position of the pyrrolidinone ring and Q bonded to the carbon at the 4-position of the ring are generally found in the trans configuration. These two carbon atoms both possess a chiral center. The most prevalent pair of enantiomers are depicted as Formula 1′ and Formula 1″. While this invention pertains to all stereoisomers, the preferred enantiomer for biological operability is identified as Formula 1′ (also referred to herein as (3S,4R) or “S,R” in the “Stereo (3,4)” column of Index Table A) where the amide (i.e. the C(O)NH(W)) moiety is projecting toward the viewer and the Q moiety is projecting away from the viewer. For a comprehensive discussion of all aspects of stereoisomerism, see Ernest L. Eliel and Samuel H. Wilen, Stereochemistry of Organic Compounds, John Wiley & Sons, 1994.

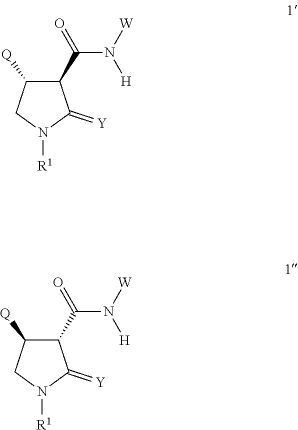

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

This invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric (>99% ee) excess of the more active isomer. Of particular note are essentially enantiomerically pure embodiments of the more active isomer.

Compounds of Formula 1 can comprise additional chiral centers. For example, substituents and other molecular constituents such as R² and R³ may themselves contain chiral centers. This invention comprises racemic mixtures as well as enriched and essentially pure stereoconfigurations at these additional chiral centers.

Compounds of this invention can exist as one or more conformational isomers due to restricted rotation about the amide bond (e.g., C(O)NH(W)) in Formula 1. This invention comprises mixtures of conformational isomers. In addition, this invention includes compounds that are enriched in one conformer relative to others.

Compounds of Formula 1 typically exist in more than one form, and Formula 1 thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a specific polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Embodiments of the present invention as described in the Summary of the Invention include (i.e. where Formula 1 as used in the following Embodiments includes N-oxides and salts thereof):
Embodiment 1. The compound of Formula 1 as described in the Summary of the Invention.
Embodiment 2. The compound of Embodiment 1 wherein Q is selected from the group consisting of Q-1 and Q-2.
Embodiment 3. The compound of Embodiment 1 wherein Q is Q-1.
Embodiment 4. The compound of any of Embodiments 1 through 3 wherein Y is O.
Embodiment 5. The compound of any of Embodiments 1 through 4 wherein R¹ is H, C₁-C₆ alkyl, C₁-C₆ haloalkyl or C₄-C₈ cycloalkylalkyl.
Embodiment 6. The compound of Embodiment 5 wherein R¹ is H, C₁-C₄ alkyl, or C₄-C₅ cycloalkylalkyl.
Embodiment 7. The compound of Embodiment 6 wherein R¹ is H, CH₃ or cyclopropylmethyl.
Embodiment 8. The compound of Embodiment 7 wherein R¹ is H or CH₃.
Embodiment 9. The compound of Embodiment 8 wherein R¹ is CH₃.
Embodiment 10. The compound of any of Embodiments 1 through 9 wherein R² is C₁-C₃ alkyl or C₁-C₃ haloalkyl.
Embodiment 11. The compound of any of Embodiments 1 through 10 wherein R² is C₁-C₂ alkyl or C₁-C₂ haloalkyl.
Embodiment 12. The compound of Embodiment 11 wherein R² is CH₃ or CH₂CF₃.
Embodiment 13. The compound of Embodiment 12 wherein R² is CH₃.
Embodiment 14. The compound of any of Embodiments 1 through 13 wherein R³ is halogen, C₁-C₆ alkyl or C₁-C₆ haloalkyl.
Embodiment 15. The compound of Embodiment 14 wherein R³ is halogen, C₁-C₃ alkyl or C₁-C₃ haloalkyl.
Embodiment 16. The compound of Embodiment 15 wherein R³ is Cl, CH₃ or CF₃.
Embodiment 17. The compound of Embodiment 16 wherein R³ is CH₃ or CF₃.
Embodiment 18. The compound of Embodiment 17 wherein R³ is CF₃.
Embodiment 19. The compound of any of Embodiments 1 through 18 wherein R⁴ is H or Cl.
Embodiment 20. The compound of Embodiment 19 wherein R⁴ is H.
Embodiment 21. The compound of any of Embodiments 1 through 20 wherein R⁵ is F, Cl or Br.
Embodiment 22. The compound of Embodiment 21 wherein R⁵ is F or Cl.
Embodiment 23. The compound of Embodiment 22 wherein R⁵ is F.
Embodiment 24. The compound of any of Embodiments 1 through 23 wherein R⁶ is H, halogen, hydroxy, C₁-C₄ alkoxy or C₁-C₄ alkyl.
Embodiment 25. The compound of Embodiment 24 wherein R⁶ is H, Cl, hydroxy, OCH₃ or CH₃.
Embodiment 26. The compound of Embodiment 25 wherein R⁶ is H.
Embodiment 27. The compound of Embodiment 25 wherein R⁶ is Cl.
Embodiment 28. The compound of Embodiment 25 wherein R⁶ is hydroxy.
Embodiment 29. The compound of Embodiment 25 wherein R⁶ is OCH₃.
Embodiment 30. The compound of Embodiment 25 wherein R⁶ is CH₃.
Embodiment 31. The compound of any of Embodiments 21 through 30 wherein n is 1, 2 or 3.
Embodiment 32. The compound of Embodiment 31 wherein n is 1 or 2.
Embodiment 33. The compound of Embodiment 32 wherein n is 1.
Embodiment 34. The compound of any of Embodiments 1 through 33 wherein W is phenyl, 3-pyridyl or 4-pyridyl, each phenyl, 3-pyridyl or 4-pyridyl optionally substituted with up to 4 R⁹.
Embodiment 35. The compound of Embodiment 34 wherein W is phenyl or 3-pyridyl, each phenyl or 3-pyridyl optionally substituted with up to 3 R⁹.
Embodiment 36. The compound of Embodiment 35 wherein W is phenyl substituted with up to 3 R⁹.
Embodiment 37. The compound of any of Embodiments 1 through 36 wherein each R⁹ is independently halogen, cyano, nitro, C₁-C₄ alkyl, C₁-C₄ haloalkyl, C₂-C₄ alkoxyalkyl, C₂-C₄ haloalkoxyalkyl, C₃-C₄ cycloalkyl, C₃-C₄ halocycloalkyl, cyclopropylmethyl, methylcyclopropyl, C₁-C₄ alkoxy, C₁-C₄ haloalkoxy, C₃-C₄ cycloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, hydroxy, formyl, C₂-C₄ alkylcarbonyl, C₂-C₄ alkylcarbonyloxy, C₁-C₄ alkylsulfonyloxy, C₁-C₄ haloalkylsulfonyloxy.
Embodiment 38. The compound of Embodiment 37 wherein each R⁹ is independently halogen, cyano, nitro, C₁-C₄ alkyl, C₁-C₄ haloalkyl, C₁-C₄ alkoxy, C₁-C₄ alkylthio, C₁-C₄ alkylsulfinyl or C₁-C₄ alkylsulfonyl.
Embodiment 39. The compound of Embodiment 38 wherein each R⁹ is independently halogen, C₁-C₄ alkyl, C₁-C₄ haloalkyl or C₁-C₄ alkylsulfonyl.
Embodiment 40. The compound of Embodiment 39 wherein each R⁹ is independently F, Cl, CH₃, CF₃ or SO₂CH₃.
Embodiment 41. The compound of Embodiment 40 wherein each R⁹ is independently F or CF₃.
Embodiment 42. The compound of any of Embodiments 34 through 41 wherein R⁹ is at the ortho, meta, or para position of W (relative to the connection to the remainder of Formula 1).
Embodiment 43. The compound of any of Embodiments 34 through 41 wherein R⁹ is at the ortho and meta position of W (relative to the connection to the remainder of Formula 1).
Embodiment 44. The compound of the Summary of the Invention wherein when R¹ is C₁-C₆ alkyl, R² is C₁-C₆ haloalkyl.

A specific embodiment of the present invention is a compound of Formula 1 wherein Y is O; R¹ is CH₃; Q is Q-1; R² is CH₃; R³ is CF₃; R⁴ is H; W is phenyl substituted with 1 R⁹ at the 2-position and 1 R⁹ at the 3-position; both R⁹ are independently F; and R⁶ is H.

This invention also includes a herbicidal mixture comprising (a) a compound selected from Formula 1, N-oxides, and salts thereof (as described in the Summary of the Invention), and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransfererase (HST) inhibitors, (b14) cellulose biosynthesis inhibitors, (b15) other herbicides including mitotic disruptors, organic arsenicals, asulam, bromobutide, cinmethylin, cumyluron, dazomet, 2-[(2,5-dichlorophenyl)methyl]-4,4-dimethyl-3-isoxazolidinone, difenzoquat, dymron, etobenzanid, flurenol, fosamine, fosamine-ammonium, hydantocidin, metam, methyldymron, oleic acid, oxaziclomefone, pelargonic acid and pyributicarb, (b16) herbicide safeners and salts of compounds of (b1) through (b16).

Embodiments of this invention, including Embodiments 1 through 44 as well as any other Embodiments described herein, can be combined in any manner, and the descriptions of variables in the embodiments pertain not only to the compounds of Formula 1 but also to the intermediate compounds useful for preparing the compounds of Formula 1. Combinations of Embodiments 1 through 44 are illustrated as follows: Embodiment A. A compound of Formula 1 wherein
R¹ is H, C₁-C₆ alkyl, C₁-C₆ haloalkyl or C₄-C₈ cycloalkylalkyl;
R² is C₁-C₃ alkyl or C₁-C₃ haloalkyl;
R³ is halogen, C₁-C₆ alkyl or C₁-C₆ haloalkyl;
Y is O;
R⁴ is H or Cl;
R⁵ is F, Cl or Br;
n is 1, 2 or 3;
R⁶ is H, halogen, hydroxy, C₁-C₄ alkoxy or C₁-C₄ alkyl;
W is phenyl, 3-pyridyl or 4-pyridyl, each phenyl, 3-pyridyl or 4-pyridyl optionally substituted with up to 4 R⁹; and
each R⁹ is independently halogen, cyano, nitro, C₁-C₄ alkyl, C₁-C₄ haloalkyl, C₂-C₄ alkoxyalkyl, C₂-C₄ haloalkoxyalkyl, C₃-C₄ cycloalkyl, C₃-C₄ halocycloalkyl, cyclopropylmethyl, methylcyclopropyl, C₁-C₄ alkoxy, C₁-C₄ haloalkoxy, C₃-C₄ cycloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, hydroxy, formyl, C₂-C₄ alkylcarbonyl, C₂-C₄ alkylcarbonyloxy, C₁-C₄ alkylsulfonyloxy, C₁-C₄ haloalkylsulfonyloxy.
Embodiment B. The compound of Embodiment A wherein
   Q is selected from the group consisting of Q-1 and Q-2;
   R¹ is H, C₄-C₅ cycloalkylalkyl or C₁-C₄ alkyl;
   R² is C₁-C₂ alkyl or C₁-C₂ haloalkyl;
   R³ is halogen, C₁-C₃ alkyl or C₁-C₃ haloalkyl;
   R⁴ is H;
   n is 1 or 2;
   R⁶ is H, Cl, hydroxy, OCH₃ or CH₃;
   W is phenyl or 3-pyridyl, each phenyl or 3-pyridyl optionally substituted with up to 3 R⁹; and
   each R⁹ is independently halogen, cyano, nitro, C₁-C₄ alkyl, C₁-C₄ haloalkyl, C₁-C₄ alkoxy, C₁-C₄ alkylthio, C₁-C₄ alkylsulfinyl or C₁-C₄ alkylsulfonyl.
Embodiment C. The compound of Embodiment B wherein
   Q is Q-1;
   R¹ is H, cyclopropylmethyl or CH₃;
   R² is CH₃ or CH₂CF₃;
   R³ is Cl, CH₃ or CF₃;
   R⁶ is H;
   W is phenyl substituted with up to 3 R⁹; and
   each R⁹ is independently halogen, C₁-C₄ alkyl, C₁-C₄ haloalkyl or C₁-C₄ alkylsulfonyl.
Embodiment D. The compound Embodiment C wherein
   R¹ is H or CH₃;
   R² is CH₃;
   R³ is CH₃ or CF₃;
   R⁶ is H;
   each R⁹ is independently F or CF₃; and
   R⁹ is at the ortho, meta, or para position of W (relative to the connection to the remainder of Formula 1).

A specific embodiment is the compound of Formula 1 that is:
(3S,4R)—N-(2,3-difluorophenyl)-1-methyl-4-[1-methyl-5-(trifluoromethyl)-1H-pyrazol-3-yl]-2-oxo-3-pyrrolidinecarboxamide.

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Compounds of the invention are particularly useful for selective control of weeds in crops such as wheat, barley, maize, soybean, sunflower, cotton, oilseed rape and rice, and specialty crops such as sugarcane, citrus, fruit and nut crops.

Also noteworthy as embodiments are herbicidal compositions of the present invention comprising the compounds of embodiments described above.

This invention also includes a herbicidal mixture comprising (a) a compound selected from Formula 1, N-oxides, and salts thereof, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransfererase (HST) inhibitors, (b14) cellulose biosynthesis inhibitors, (b15) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b16) herbicide safeners, and salts of compounds of (b1) through (b16).

“Photosystem II inhibitors” (b1) are chemical compounds that bind to the D-1 protein at the Q_{B}-binding niche and thus block electron transport from Q_{A} to Q_{B} in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The Q_{B}-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem II inhibitors include ametryn, amicarbazone, atrazine, bentazon, bromacil, bromofenoxim, bromoxynil, chlorbromuron, chloridazon, chlorotoluron, chloroxuron, cumyluron, cyanazine, daimuron, desmedipham, desmetryn, dimefuron, dimethametryn, diuron, ethidimuron, fenuron, fluometuron, hexazinone, ioxynil, isoproturon, isouron, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryn, tebuthiuron, terbacil, terbumeton, terbuthylazine, terbutryn and trietazine.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl, bispyribac-sodium, cloransulam-methyl, chlorimuron-ethyl, chlorsulfuron, cinosulfuron, cyclosulfamuron, diclosulam, ethametsulfuron-methyl, ethoxysulfuron, flazasulfuron, florasulam, flucarbazone-sodium, flumetsulam, flupyrsulfuron-methyl, flupyrsulfuron-sodium, foramsulfuron, halosulfuron-methyl, imazamethabenz-methyl, imazamox, imazapic, imazapyr, imazaquin, imazethapyr, imazosulfuron, iodosulfuron-methyl (including sodium salt), iofensulfuron (2-iodo-N-[[(4-methoxy-6-methyl-1,3,5-triazin-2-yl)amino]carbonyl]benzenesulfonamide), mesosulfuron-methyl, metazosulfuron (3-chloro-4-(5,6-dihydro-5-methyl-1,4,2-dioxazin-3-yl)-N-[[(4,6-dimethoxy-2-pyrimidinyl)amino]carbonyl]-1-methyl-1H-pyrazole-5-sulfonamide), metosulam, metsulfuron-methyl, nicosulfuron, oxasulfuron, penoxsulam, primisulfuron-methyl, propoxycarbazone-sodium, propyrisulfuron (2-chloro-N-[[(4,6-dimethoxy-2-pyrimidinyl)amino]carbonyl]-6-propylimidazo [1,2-b]pyridazine-3-sulfonamide), prosulfuron, pyrazosulfuron-ethyl, pyribenzoxim, pyriftalid, pyriminobac-methyl, pyrithiobac-sodium, rimsulfuron, sulfometuron-methyl, sulfosulfuron, thiencarbazone, thifensulfuron-methyl, triafamone (N-[2-[(4,6-dimethoxy-1,3,5-triazin-2-yl)carbonyl]-6-fluorophenyl]-1,1-difluoro-N-methylmethanesulfonamide), triasulfuron, tribenuron-methyl, trifloxysulfuron (including sodium salt), triflusulfuron-methyl and tritosulfuron.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl CoA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include alloxydim, butroxydim, clethodim, clodinafop, cycloxydim, cyhalofop, diclofop, fenoxaprop, fluazifop, haloxyfop, pinoxaden, profoxydim, propaquizafop, quizalofop, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b4) are chemical compounds mimicking the plant growth hormone auxin, thus causing uncontrolled and disorganized growth leading to plant death in susceptible species. Examples of auxin mimics include aminocyclopyrachlor (6-amino-5-chloro-2-cyclopropyl-4-pyrimidinecarboxylic acid) and its methyl and ethyl esters and its sodium and potassium salts, aminopyralid, benazolin-ethyl, chloramben, clacyfos, clomeprop, clopyralid, dicamba, 2,4-D, 2,4-DB, dichlorprop, fluroxypyr, halauxifen (4-amino-3-chloro-6-(4-chloro-2-fluoro-3-methoxyphenyl)-2-pyridinecarboxylic acid), halauxifen-methyl (methyl 4-amino-3-chloro-6-(4-chloro-2-fluoro-3-methoxyphenyl)-2-pyridinecarboxylate), MCPA, MCPB, mecoprop, picloram, quinclorac, quinmerac, 2,3,6-TBA, triclopyr, and methyl 4-amino-3-chloro-6-(4-chloro-2-fluoro-3-methoxyphenyl)-5-fluoro-2-pyridinecarboxylate.

“EPSP synthase inhibitors” (b5) are chemical compounds that inhibit the enzyme 5-enol-pyruvylshikimate-3-phosphate synthase, which is involved in the synthesis of aromatic amino acids such as tyrosine, tryptophan and phenylalanine. EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosystem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include diquat and paraquat.

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, azafenidin, benzfendizone, bifenox, butafenacil, carfentrazone, carfentrazone-ethyl, chlomethoxyfen, cinidon-ethyl, fluazolate, flufenpyr-ethyl, flumiclorac-pentyl, flumioxazin, fluoroglycofen-ethyl, fluthiacet-methyl, fomesafen, halosafen, lactofen, oxadiargyl, oxadiazon, oxyfluorfen, pentoxazone, profluazol, pyraclonil, pyraflufen-ethyl, saflufenacil, sulfentrazone, thidiazimin, trifludimoxazin (dihydro-1,5-dimehyl-6-thioxo-3-[2,2,7-trifluoro-3,4-dihydro-3-oxo-4-(2-propyn-1-yl)-2H-1,4-benzoxazin-6-yl]-1,3,5-triazine-2,4(1H,3H)-dione) and tiafenacil (methyl N-[2-[[2-chloro-5-[3,6-dihydro-3-methyl-2,6-dioxo-4-(trifluoromethyl)-1(2H)-pyrimidinyl]-4-fluorophenyl]thio]-1-oxopropyl]-β-alaninate).

“GS inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P ((2S)-2-amino-4-(hydroxymethylphosphinyl)butanoic acid) and bilanaphos.

“very long chain fatty acid (VLCFA) elongase inhibitors” (b9) are herbicides having a wide variety of chemical structures, which inhibit the elongase. Elongase is one of the enzymes located in or near chloroplasts which are involved in biosynthesis of very long chain fatty acids. In plants, very-long-chain fatty acids are the main constituents of hydrophobic polymers that prevent desiccation at the leaf surface and provide stability to pollen grains. Such herbicides include acetochlor, alachlor, anilofos, butachlor, cafenstrole, dimethachlor, dimethenamid, diphenamid, fenoxasulfone (3-[[(2,5-dichloro-4-ethoxyphenyl)methyl]sulfonyl]-4,5-dihydro-5,5-dimethylisoxazole), fentrazamide, flufenacet, indanofan, mefenacet, metazachlor, metolachlor, naproanilide, napropamide, napropamide-M ((2R)—N,N-diethyl-2-(1-naphthalenyloxy)propanamide), pethoxamid, piperophos, pretilachlor, propachlor, propisochlor, pyroxasulfone, and thenylchlor, including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include diflufenzopyr, naptalam (also known as N-(1-naphthyl)phthalamic acid and 2-[(1-naphthalenylamino)carbonyl]benzoic acid).

“PDS inhibitors” (b11) are chemical compounds that inhibit carotenoid biosynthesis pathway at the phytoene desaturase step. Examples of PDS inhibitors include beflubutamid, beflubutamid-M, diflufenican, fluridone, fluorochloridone, flurtamone, norflurazon and picolinafen.

“HPPD inhibitors” (b12) are chemical substances that inhibit the biosynthesis of synthesis of 4-hydroxyphenyl-pyruvate dioxygenase. Examples of HPPD inhibitors include benzobicyclon, benzofenap, bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one), fenquinotrione (2-[[8-chloro-3,4-dihydro-4-(4-methoxyphenyl)-3-oxo-2-quinoxalinyl]carbonyl]-1,3-cyclohexanedione), isoxachlortole, isoxaflutole, mesotrione, pyrasulfotole, pyrazolynate, pyrazoxyfen, sulcotrione, tefuryltrione, tembotrione, tolpyralate (1-[[1-ethyl-4-[3-(2-methoxyethoxy)-2-methyl-4-(methylsulfonyl)benzoyl]-1H-pyrazol-5-yl]oxy]ethyl methyl carbonate), topramezone, 5-chloro-3-[(2-hydroxy-6-oxo-1-cyclohexen-1-yl)carbonyl]-1-(4-methoxyphenyl)-2(1H)-quinoxalinone, 4-(2,6-diethyl-4-methylphenyl)-5-hydroxy-2,6-dimethyl-3(2H)-pyridazinone, 4-(4-fluorophenyl)-6-[(2-hydroxy-6-oxo-1-cyclohexen-1-yl)carbonyl]-2-methyl-1,2,4-triazine-3,5(2H,4H)-dione, 5-[(2-hydroxy-6-oxo-1-cyclohexen-1-yl)carbonyl]-2-(3-methoxyphenyl)-3-(3-methoxypropyl)-4(3H)-pyrimidinone, 2-methyl-N-(4-methyl-1,2,5-oxadiazol-3-yl)-3-(methylsulfinyl)-4-(trifluoromethyl)benzamide and 2-methyl-3-(methylsulfonyl)-N-(1-methyl-1H-tetrazol-5-yl)-4-(trifluoromethyl)benzamide.

“HST inhibitors” (b13) disrupt a plant's ability to convert homogentisate to 2-methyl-6-solanyl-1,4-benzoquinone, thereby disrupting carotenoid biosynthesis. Examples of HST inhibitors include haloxydine, pyriclor, 3-(2-chloro-3,6-difluorophenyl)-4-hydroxy-1-methyl-1,5-naphthyridin-2(1H)-one, 7-(3,5-dichloro-4-pyridinyl)-5-(2,2-difluoroethyl)-8-hydroxypyrido[2,3-b]pyrazin-6(5H)-one and 4-(2,6-diethyl-4-methylphenyl)-5-hydroxy-2,6-dimethyl-3(2H)-pyridazinone.

HST inhibitors also include compounds of Formulae A and B.

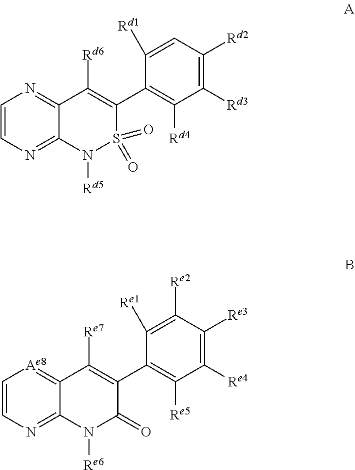

wherein R^{d1} is H, Cl or CF₃; R^{d2} is H, Cl or Br; R^{d3} is H or Cl; R^{d4} is H, Cl or CF₃; R^{d5} is CH₃, CH₂CH₃ or CH₂CHF₂; and R^{d6} is OH, or —OC(═O)-i-Pr; and R^{e1} is H, F, Cl, CH₃ or CH₂CH₃; R^{e2} is H or CF₃; R^{e3} is H, CH₃ or CH₂CH₃; R^{e4} is H, F or Br; R^{e5} is Cl, CH₃, CF₃, OCF₃ or CH₂CH₃; R^{e6} is H, CH₃, CH₂CHF₂ or C═CH; R^{e7} is OH, —OC(═O)Et, —OC(═O)-i-Pr or —OC(═O)-t-Bu; and A^{e8} is N or CH.

“Cellulose biosynthesis inhibitors” (b14) inhibit the biosynthesis of cellulose in certain plants. They are most effective when applied preemergence or early postemergence on young or rapidly growing plants. Examples of cellulose biosynthesis inhibitors include chlorthiamid, dichlobenil, flupoxam, indaziflam (N²-[(1R,2S)-2,3-dihydro-2,6-dimethyl-1H-inden-1-yl]-6-(1-fluoroethyl)-1,3,5-triazine-2,4-diamine), isoxaben and triaziflam.

“Other herbicides” (b15) include herbicides that act through a variety of different modes of action such as mitotic disruptors (e.g., flamprop-M-methyl and flamprop-M-isopropyl), organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b14) or act through a combination of modes of action listed above. Examples of other herbicides include aclonifen, asulam, amitrole, bixlozone, bromobutide, cinmethylin, clomazone, cumyluron, cyclopyrimorate (6-chloro-3-(2-cyclopropyl-6-methylphenoxy)-4-pyridazinyl 4-morpholinecarboxylate), daimuron, difenzoquat, etobenzanid, fluometuron, flurenol, fosamine, fosamine-ammonium, dazomet, dymron, ipfencarbazone (1-(2,4-dichlorophenyl)-N-(2,4-difluorophenyl)-1,5-dihydro-N-(1-methylethyl)-5-oxo-4H-1,2,4-triazole-4-carboxamide), metam, methyldymron, oleic acid, oxaziclomefone, pelargonic acid, pyributicarb and 5-[[(2,6-difluorophenyl)methoxy]methyl]-4,5-dihydro-5-methyl-3-(3-methyl-2-thienyl)isoxazole.

“Herbicide safeners” (b16) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide and N-(aminocarbonyl)-2-fluorobenzenesulfonamide, 1-bromo-4-[(chloromethyl)sulfonyl]benzene, 2-(dichloromethyl)-2-methyl-1,3-dioxolane (MG 191), 4-(dichloroacetyl)-1-oxa-4-azospiro[4.5]decane (MON 4660), 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

The compounds of Formula 1 can be prepared by general methods known in the art of synthetic organic chemistry. One or more of the following methods and variations as described in Schemes 1-19 can be used to prepare the compounds of Formula 1. The definitions of Q, R¹, R², R³, Y, R⁴, R⁵, n, R⁶, W and R⁹ in the compounds of Formulae 1-28 below are as defined above in the Summary of the Invention unless otherwise noted. Compounds of Formulae 1A, 1B, 1C, 4A, 5A, 5A′, 5A″, 5C and 8A are subsets of the compounds of Formula 1, and all substituents for Formulae 1A, 1B, 1C, 4A, 5A, 5A′, 5A″, 5C and 8A are as defined above for Formula 1 unless otherwise noted.

As shown in Scheme 1 a compound of Formula 1A (i.e. a compound of Formula 1 wherein Y is O) can be prepared by reaction of acids of Formula 2 with an amine of Formula 3 in the presence of a dehydrative coupling reagent such as propylphosphonic anhydride, dicyclohexylcarbodiimide, N-(3-dimethylaminopropyl)-N-ethylcarbodiimide, N,N-carbonyldiimidazole, 2-chloro-1,3-dimethylimidazolium chloride or 2-chloro-1-methylpyridinium iodide. Polymer-supported reagents, such as polymer-supported cyclohexylcarbodiimide, are also suitable. These reactions are typically run at temperatures ranging from 0-60° C. in a solvent such as dichloromethane, acetonitrile, N,N-dimethylformamide or ethyl acetate in the presence of a base such as triethylamine, N,N-diisopropylamine, or 1,8-diazabicyclo[5.4.0]undec-7-ene. See Organic Process Research & Development 2009, 13, 900-906 for coupling conditions employing propylphosphonic anhydride.

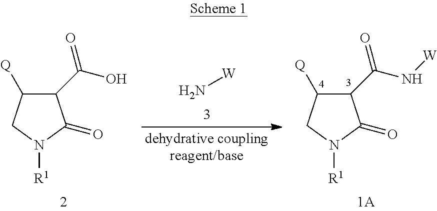

As shown in Scheme 2, compounds of Formula 2 can be prepared by hydrolysis of esters of Formula 4 by methods known to those skilled in the art. Hydrolysis is carried out with aqueous base or aqueous acid, typically in the presence of a co-solvent. Suitable bases for the reaction include, but are not limited to, hydroxides such as sodium and potassium hydroxide and carbonates such as sodium and potassium carbonate. Suitable acids for the reaction include, but are not limited to, inorganic acids such as hydrochloric acid, hydrobromic acid and sulfuric acid, and organic acids such as acetic acid and trifluoroacetic acid. A wide variety of co-solvents are suitable for the reaction including, but not limited to, methanol, ethanol and tetrahydrofuran. The reaction is conducted at temperatures ranging from −20° C. to the boiling point of the solvent, and typically from 0 to 100° C. Additionally, compounds of Formula 2 where R¹ is H can be further converted to compounds of Formula 2 where R¹ is alkyl, haloalkyl, cycloalkyl, cycloalkylalkyl by an alkylation reaction. A variety of bases and alkylating agents are possible, but a preferred method is by treating the compound of Formula 2 (where R¹ is H) with excess potassium-tert-butoxide in tetrahydrofuran at 0° C. and adding the alkylating reagent.

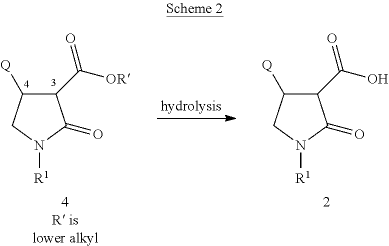

As shown in Scheme 3, a compound of Formula 4A (a compound of Formula 4 wherein R¹ is H) can be obtained by reduction of a compound of Formula 5 and subsequent in situ cyclization of the resulting intermediate amine. A wide variety of methods for reduction of the aliphatic nitro group in compounds of Formula 5 are known in the literature. Methods known to those skilled in the art include catalytic hydrogenation in the presence of palladium on carbon or Raney nickel, iron or zinc metal in acidic medium (see, for example, Berichte der Deutschen Chemischen Gesellschaft 1904, 37, 3520-3525), and lithium aluminum hydride. Reduction of an aliphatic nitro group can also be achieved with samarium(II) iodide in the presence of a proton source such as methanol (see for example, Tetrahedron Letters 1991, 32 (14), 1699-1702). Alternatively sodium borohydride in the presence of a nickel catalyst such as nickel(II) acetate or nickel(II) chloride can be used (see for example, Tetrahedron Letters 1985, 26 (52), 6413-6416).

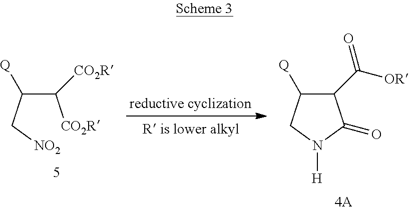

As shown in Scheme 4, a compound of Formula 5 can be prepared by reacting diesters of Formula 6 with nitroalkanes of Formula 7, typically in the presence of a base. Suitable bases for the reaction include alkali metal lower alkoxides such as sodium methoxide in methanol or sodium ethoxide in ethanol. Preferrably the diester compound of Formula 6 and the lower alkoxide bases are derived from the same alcohol. Compounds of Formula 6 can be prepared by methods known to those skilled in the art, e.g., by Knoevenagel condensation of aldehydes and malonates (see, for example, G. Jones, Organic Reactions Volume 15, John Wiley and Sons, 1967).

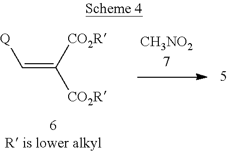

Compounds of Formula 5A can be prepared by reacting compounds of Formula 8 with malonates of Formula 9 in the presence of a base as shown in Scheme 5. Suitable bases for this reaction include, but are not limited to, alkali metal lower alkoxides such as sodium methoxide in methanol or sodium ethoxide in ethanol, or bases such as lithium bis(trimethylsilyl)amide, sodium bis(trimethylsilyl)amide and lithium diisopropylamide in solvents such as tetrahydrofuran. Typically, the reaction is carried out in the range of from −78° C. to 23° C. See Synthesis 2005, 2239-2245 for conditions for effecting this transformation. Conditions for effecting this transformation in refluxing water in the absence of a catalyst are reported in Synthetic Communications 2013, 43, 744-748.

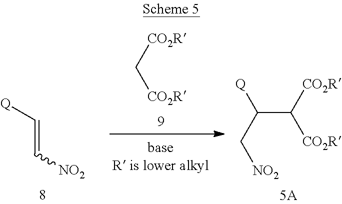

Compounds of Formula 6 can be prepared by Knoevenagel condensation of aldehydes of Formula 14 and malonates of Formula 9 as shown in Scheme 6. Also shown in Scheme 6, a compound of Formula 8 can be prepared by Knoevenagel condensation of aldehydes of Formula 14 and nitromethane.

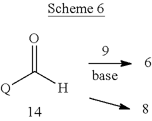

Compounds of Formulae 5A′ and 5A″ can be prepared stereoselectively by reacting nitroalkanes of Formula 8 with malonates of Formula 9 in the presence of a chiral catalyst and optionally in the presence of a suitable base as shown in Scheme 7. Suitable catalysts include, but are not limited to Ni(II) with vicinal diamine ligands such as Ni(II) bis[(R,R)—N,N′-dibenzylcyclohexane-1,2-diamine]dibromide, Ni(II) bis[(S,S)—N,N-dibenzylcyclohexane-1,2-diamine]dibromide or nickel(II) bromide with chiral 1,1′-bi(tetrahydroisoquinoline) type diamines. Suitable organic bases for this reaction include, but are not limited to, piperidine, morpholine, triethylamine, 4-methylmorpholine or N,N-diisopropylethylamine. This transformation can be accomplished neat or in solvents such as tetrahydrofuran, toluene or dichloromethane. Typically, the reaction is carried out in the range of from −78° C. to 80° C. using 0 to 1 equivalent of catalyst and optionally 0 to 1 equivalent of a base. Conditions for effecting this transformation have been reported in J. Am. Chem. Soc. 2005, 9958-9959 or Eur. J. Org. Chem. 2011, 5441-5446 for conditions. Nitroalkenes of Formula 8 can be prepared from aldehydes and nitromethane by methods known to those skilled in the art.

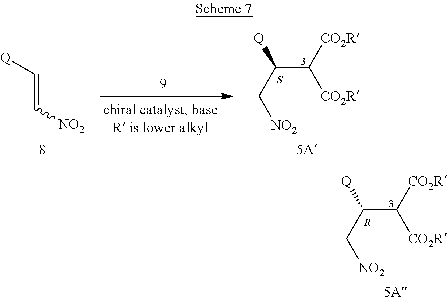

As shown in Scheme 8, a compound of Formula 10 can be prepared by reacting a compound of Formula 11 with a compound of Formula 7 in a solvent, in the presence of a base analogous to the method described in Scheme 4.

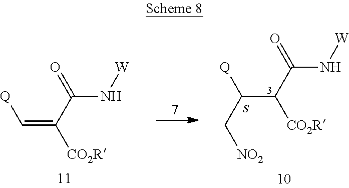

As shown in Scheme 9, a compound of Formula 10 can be prepared, analogous to the method of Scheme 5, by reacting a nitroalkane of Formula 8 with a compound of Formula 12.

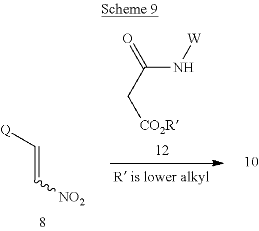

As shown in Scheme 10, a compound of Formula 11A can be prepared by reaction of malonic amide of Formula 12 with an aldehyde of Formula 14 by methods known to those skilled in the art. Also shown in Scheme 10, malonic amides of Formula 12 can readily be prepared from lower alkyl malonyl chlorides of Formula 13 such as methyl malonyl chloride and amines of Formula 3 by methods known to those skilled in the art.

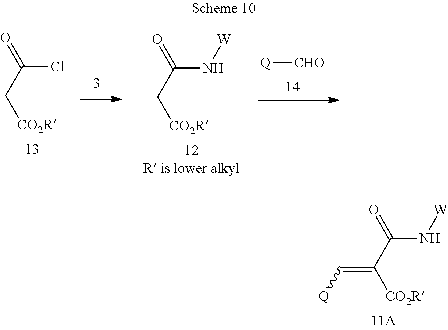

As shown in Scheme 11, a compound of Formula 1B can be produced by reaction of a compound of Formula 15 with an isocyanate of Formula 16 in the presence of a base. Examples of a base which can be used for the present process include those listed for the method of Scheme 4. The reaction temperature can be selected from the range of from −78° C. to the boiling point of the inert solvent used. Typically, the reaction is carried out at temperatures ranging from −78° C. to 100° C. in solvents such as toluene.

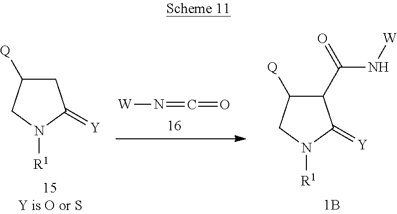

As shown in Scheme 12, a compound of Formula 15 can be prepared by reaction of a compound of Formula 17 with corresponding electrophiles of Formula 18 in the presence of base. In Formula 18, G denotes a leaving group, i.e. a nucleofuge. Depending upon the selection of R¹, suitable electrophiles for the reaction can include alkyl halides such as chlorides, bromides and iodides, alkylsulfonates. Suitable bases for the reaction include inorganic bases such as alkali or alkaline earth metal (e.g., lithium, sodium, potassium and cesium) hydroxides, alkoxides, carbonates, and phosphates, and organic bases such as triethylamine, N,N-diisopropylethylamine and 1,8-diazabicyclo[5.4.0]undec-7-ene. A wide variety of solvents are suitable for the reaction including, but not limited to, tetrahydrofuran, dichloromethane, N,N-dimethylformamide, N,N-dimethylacetamide, N-methylpyrrolidinone, acetonitrile, C₂-C₆ alcohols and acetone as well as mixtures of these solvents. This reaction is conducted at temperatures ranging from −20 to 200° C., and typically between 0 and 50° C.

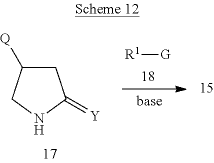

As shown in Scheme 13, a compound of Formula 17 can be prepared by decarboxylation of an acid of Formula 2 by methods known to those skilled in the art. Decarboxylation is carried out by heating a compound of Formula 2 in a solvent, typically in the presence of an acid. Suitable acids for the reaction include, but are not limited to, p-toluenesulfonic acid. A wide variety of co-solvents are suitable for the reaction including, but not limited to, toluene, isopropyl acetate and methyl isobutylketone. The reaction is conducted at temperatures ranging from −20° C. to the boiling point of the solvent, and typically from 0 to 150° C.

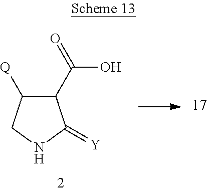

As shown in Scheme 14, a compound of Formula 1C (i.e. a compound of Formula 1 wherein R¹ is H, and Y is S) can be prepared by reacting a compound of Formula 1A with at least one equivalent of a thionation reagent such as Lawesson's reagent, tetraphosphorus decasulfide or diphosphorus pentasulfide in a solvent such as tetrahydrofuran or toluene. Typically, the reaction is carried out at temperatures ranging from 0 to 115° C.

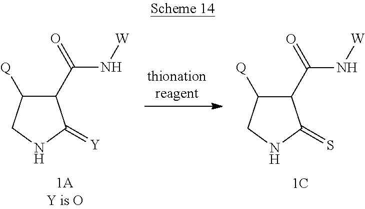

Compounds of Formula 8 can also be prepared from the reaction of nitroenamines of Formula 19 with Grignard or lithium reagents of Formula 20 as detailed in Scheme 15. See Severin in Chem. Ber. 1969, 102, 2966-71 for examples of this reaction and conditions. The Grignard and lithium reagents may be made by halogen metal exchange reactions on known or commercially available bromides and iodides.

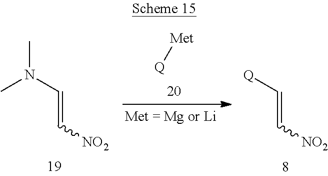

Aldehydes of Formula 14 and halides (precursors to prepare a compound of Formula 20) used as starting materials for compounds of Formulae 6 and 8 are commercially available or known to those skilled in the art. A useful method for the synthesis of starting compounds when Q is Q-1 and where R⁴ is alkyl or haloalkyl is found in J. Heterocyclic Chem. 1989, 26, 895-98. A review of methods for the synthesis of pyrazole starting materials wherein Q is Q-1 or Q-2 is found in Chem. Rev. 2011, 111, 6984-7034 and references cited therein. Useful methods for synthesis of indazole compounds wherein Q is Q-3 can be found in World Patent Applications WO 2011/050245 and WO 2018/177781 and in Molecules 2018, 23(11), 2783 and references cited therein. Metallation reactions of pyrazoles to functionalize them on the 3-, 4-, and 5-positions have been detailed in the thesis of Christina Despotopoulou, University of Munich (LMU), 2009 and references cited therein.

Synthesis of a compound of Formula 14 wherein Q is Q-1 and R³ is alkyl or haloalkyl is shown in Scheme 16. A compound of Formula 21 may be reacted with a compound of Formula 22 in the presence of base to afford intermediates of Formula 23. A compound of Formula 23 may in turn be cyclized by reaction with alkyl hydrazines to form pyrazolines (when R³ is haloalkyl) and pyrazoles (when R³ is alkyl) which may be hydrolyzed and dehydrated in one step using aqueous acid to provide compounds of Formula 14 wherein Q is Q-1. The base used in the cyclization may be, but not limited to alkali hydroxides and lower alkoxides such as sodium methoxide, potassium and sodium tert-butoxide, alkali hydrides such as sodium hydride, sodium hexamethyldisilazide, potassium hexamethyldisilazide, and lithium hexamethyldisilazide. Aqueous acids such as but not limited to hydrochloric, sulfuric, acetic and trifluoroacetic acids are suitable for the hydrolysis/dehydration step.

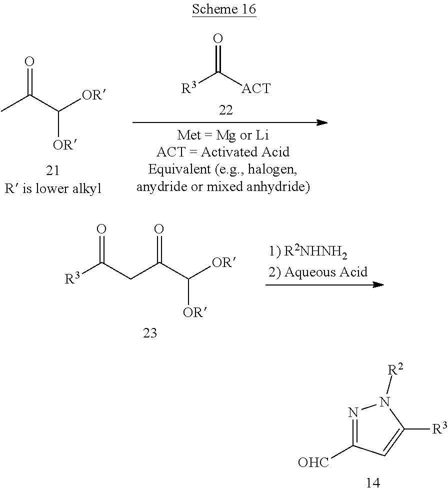

An alternative route to the synthesis of a compound of Formula 4 is shown in Scheme 17. Cycloaddition of compounds of Formula 24 with acrylates of Formula 25 leads to the pyrrolidinone ring system with a protected thiocarbonyl (e.g., a compound of Formula 26). The thiocarbonyl can be hydrolyzed to the carbonyl under oxidizing conditions with reagents such as oxone and hydrogen peroxide. See Fishwick, Tet. Lett. 1995, 36, 9409-9412 and citing documents such as Eur. J. Org. Chem. 2001, 3533-3544 for conditions to carry out these cycloadditions and methods to synthesize a compound of Formula 24. Acrylates of Formula 25 can readily be made by Wittig or Horner-Emmons Wadsworth reactions of aldehydes of Formula 14 or by the Heck reactions of the corresponding halogenated materials.

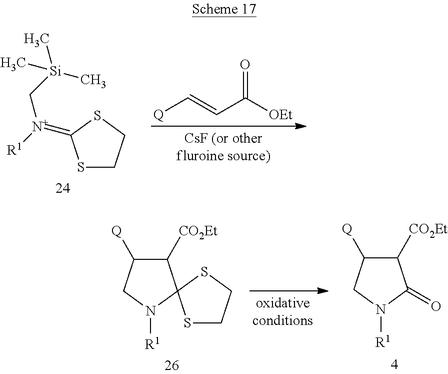

Another useful method for synthesis of a compound of Formula 1 is shown in Scheme 18. Rhodium catalyzed addition of boronic acids of Formula 27 to unsaturated pyrrolidinones of Formula 28 directly leads to a compound of Formula 1 in the presence of a rhodium catalyst (Hayashi Reaction). A wide variety of conditions and catalysts for carrying out Hayashi reactions are provided by Frost et al. in Chem. Soc. Rev. 2010, 39, 2093-2105 and references therein. This review article also describes conditions for providing enantiomerically pure products. The unsaturated pyrrolidinones of Formula 28 can be made from well-known saturated pyrrolidinones by many types of dehydrogenation conditions for example phenylselenide elimination.

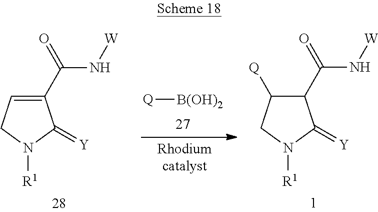

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “br” means broad, and “br s” means broad singlet. Mass spectra (MS) are reported as the molecular weight of the highest isotopic abundance parent ion (M+1) formed by addition of H+ (molecular weight of 1) to the molecule, or (M−1) formed by the loss of H+(molecular weight of 1) from the molecule, observed by using liquid chromatography coupled to a mass spectrometer (LCMS) using either atmospheric pressure chemical ionization (AP+) where “amu” stands for unified atomic mass units.

### Synthesis Example 1

### Preparation of (3S,4R)—N-(2,3-difluorophenyl)-1-methyl-4-[1-methyl-5-(trifluoromethyl)-1H-pyrazol-3-yl]-2-oxo-3-pyrrolidinecarboxamide (Compound 13)

### Step A: Preparation of 4,4-diethoxy-1,1,1-trifluoro-3-buten-2-one

To a solution of triethyl orthoacetate (450 g, 2.77 mol) and pyridine (550 g, 6.9 mol) in dichloromethane (3500 mL) at 0° C. was added trifluoroacetic anhydride (1135 g, 5.54 mol) dropwise. The reaction mixture was stirred at ambient temperature overnight, then quenched with cold saturated NaHCO₃ solution, then washed with water. The organic layer was dried over sodium sulfate concentrated under reduced pressure and dried under vacuum to afford the title compound as an oil (500 g, 85%).

¹H NMR (300 MHz, CDCl₃) δ 4.94 (s, 1H), 4.37 (q, 2H), 4.15 (q, 2H), 1.46 (t, 3H), 1.42 (t, 3H). LC-MS (ESI) m/z 213 (M+H)⁺.

### Step B: Preparation of (3E)-4-amino-4-ethoxy-1,1,1-trifluoro-3-buten-2-one

To a solution of 4,4-diethoxy-1,1,1-trifluoro-3-buten-2-one (i.e. the product obtained in Step A, 500 g, 2.35 mol) in acetonitrile (2500 mL) at room temperature was added dropwise 28% solution of NH₄OH in water (500 mL). The reaction mixture was stirred at ambient temperature overnight. The solvent was removed under reduced pressure, and dichloromethane was added, then washed with water. The organic layer was dried over sodium sulfate and concentrated under reduced pressure to afford the title compound as a solid (325 g, 75%).

¹H NMR (300 MHz, CDCl₃) δ 9.79 (br, 1H), 5.66 br, 1H), 5.13 (s, 1H), 4.15 (q, 2H), 1.38 (t, 3H). LC-MS (ESI) m/z 184 (M+H)⁺.

### Step C: Preparation of 1-methyl-5-(trifluoromethyl)-1H-pyrazol-3-amine

To a suspension of (3E)-4-amino-4-ethoxy-1,1,1-trifluoro-3-buten-2-one (i.e. the product obtained in Step B, 325 g, 1.76 mol) and methylhydrazine sulfate (1:1, 299 g, 2.11 mol) in ethanol (1500 mL) was added triethylamine (285 g, 2.8 mol) at ambient temperature. The reaction mixture was heated and stirred at 95° C. for 7 h. The reaction mixture was concentrated under reduced pressure and the resulting residue was diluted with water and made basic with NaHCO₃ solution and extracted with dichloromethane. The organic extracts were dried over sodium sulphate then concentrated under reduced pressure. The resulting crude product was purified by silica gel chromatography eluting with 5 to 25% ethyl acetate/hexane as eluents to afford to afford the title compound as a brown low-melting solid (75 g, 25%).

¹H NMR (300 MHz, CDCl₃) δ 5.94 (s, 1H), 3.78 (s, 3H), 3.67 (br, 2H). LC-MS (ESI) m/z 166 (M+H)⁺.

### Step D: Preparation of 3-iodo-1-methyl-5-(trifluoromethyl)-1H-pyrazole

To a solution of 1-methyl-5-(trifluoromethyl)-1H-pyrazol-3-amine (75 g, 0.454 mol) in hydrochloric acid (concentrated, 750 mL) was added dropwise a solution of sodium nitrite (38 g, 0.545 mol) dissolved in water (50 mL) at −10° C. The reaction mixture was stirred at −10° C. for 30 min then a solution of potassium iodide (162 g, 0.98 mol) in water (200 mL) was added dropwise at −10° C. The reaction mass was slowly brought to ambient temperature during over 1 h. The reaction mixture was diluted with water and dichloromethane. A saturated solution of sodium thiosulfate was added which resulted in a clear solution. The organic layer was separated, dried over sodium sulfate and concentrated under reduced pressure. The crude product was purified by silica gel chromatography eluting with 5 to 10% ethyl acetate in hexanes to provide the title compound as a light yellow liquid (70 g, 55%).

¹H NMR (400 MHz, CDCl₃) δ 4.01 (d, J=0.61 Hz, 3H) 6.76 (s, 1H). LC-MS (ESI) m/z 277 (M+H)⁺.

### Step E: Preparation of 1-methyl-3-[(1E)-2-nitroethenyl]-5-(trifluoromethyl)-1H-pyrazole

Isopropylmagnesium chloride-lithium chloride complex (1.3 M solution in tetrahydrofuran, 293 mL, 0.382 mmol) was added dropwise to 3-iodo-1-methyl-5-(trifluoromethyl)-1H-pyrazole (i.e. the product of Step D, 70 g, 0.254 mol) in tetrahydrofuran (700 mL) at −20° C. and stirred for 2 h at the same temperature. A solution of 1-(dimethylamino)-2-nitroethylene (44.5 g 0.382 mol) in tetrahydrofuran (200 mL) was added and the reaction was slowly warmed to room temp over a period of 1 h. The reaction was carefully quenched with aqueous hydrochloric acid (2 M), then stirred for 1 h and extracted with ethyl acetate. The organic layers were washed with brine, dried over sodium sulfate, filtered and concentrated onto silica gel for purification by silica gel chromatography eluting with 10% ethyl acetate/petroleum ether to provide the title compound as a yellow oil (35 g, 62%).

¹H NMR (400 MHz, CDCl₃) δ 7.89 (d, J=13.7 Hz, 1H), 7.63 (d, J=13.7 Hz, 1H), 6.88 (s, 1H), 4.05 (d, J=0.6 Hz, 3H). LC-MS (ESI) m/z 222 (M+H)⁺.

### Step F: Preparation of 1,3-diethyl 2-[(1S)-1-[1-methyl-5-(trifluoromethyl)-1H-pyrazol-3-yl]-2-nitroethyl]propanedioate

To a solution of 1-methyl-3-[(1E)-2-nitroethenyl]-5-(trifluoromethyl)-1H-pyrazole (i.e. the product obtained in Step E, 35 g, 0.158 mol) in toluene (100 mL) was added diethyl malonate (32.8 g 0.205 mol) followed by nickel bis[(1R,2R)—N1,N2-Bis(phenylmethyl)-1,2-cyclohexanediamine-N1,N2]dibromo-(OC-6-12)-2 (0.02 eq, 2.5 g, 3.16 mmol), and the mixture was stirred at ambient temp for 16 h. The reaction mixture was then concentrated under reduced pressure and the resulting residue was purified by column chromatography eluting with 25% ethyl acetate/petroleum ether to get title compound as pale pinkish-yellow oil (55 g, 92%).

¹H NMR: (400 MHz, CDCl₃) δ 6.53 (s, 1H), 5.01 (dd, 1H), 4.88 (dd, J=4.3, 13.9 Hz, 1H), 4.35 (dd, J=4.4, 7.7, 9.0 Hz, 1H), 4.22 (q, 2H), 4.16 (q, J=7.1 Hz, 2H), 3.90 (s, 3H), 3.89 (d, 1H), 1.26 (t, 3H), 1.20 (t, J=7.2 Hz, 3H). LC-MS (ESI) m/z 382 (M+H)⁺.

### Step G: Preparation of ethyl (3R,4R)-4-[1-methyl-5-(trifluoromethyl)-1H-pyrazol-3-yl]-2-oxo-3-pyrrolidinecarboxylate

To a solution of 1,3-diethyl 2-[(1S)-1-[1-methyl-5-(trifluoromethyl)-1H-pyrazol-3-yl]-2-nitroethyl]propanedioate (i.e the product obtained in Step F, 55 g, 21.9 mmol) in ethanol (500 mL) at 0-5° C. (ice bath) under nitrogen was added nickel(II) chloride hexahydrate (5.45 g, 22.9 mmol). Then sodium borohydride (2.5 g, 65.7 mmol) was then added portionwise (with effervescence) to the pale greenish-blue solution. The reaction mixture turned black as soon as the first portion of sodium borohydride was added. After 30 min the cooling was removed and the reaction mixture was allowed to warm to ambient temperature. The reaction mixture was stirred at ambient temperature for a further 3 h. The reaction mixture was cooled to around 5-10° C. in an ice-water bath, and slowly quenched with NH₄Cl solution. The mixture was diluted with ethyl acetate (2000 mL) and filtered through a bed of Celite® diatomaceous earth filter aid, washing through with portions of water and ethyl acetate. The organic layer was separated, washed with water, saturated brine solution, dried over sodium sulfate and concentrated under reduced pressure. The residue was purified by column chromatography eluting with 50% ethyl acetate/petroleum ether to get the title compound as a yellow oil (23 g, 52%).

¹H NMR: (400 MHz, CDCl₃) δ 6.91 (br s, 1H), 6.47 (s, 1H), 4.28 (q, J=7.2 Hz, 2H), 4.14 (q, 1H), 3.94 (d, 3H), 3.80 (d, J=1.0, 9.0 Hz, 1H), 3.63 (d, J=9.3 Hz, 1H), 3.52 (dd, J=8.2, 9.5 Hz, 1H), 1.32 (t, J=7.2 Hz, 3H). LC-MS (ESI) m/z 306 (M+H)⁺.

### Step H: Preparation of (3R,4R)-4-[1-methyl-5-(trifluoromethyl)-1H-pyrazol-3-yl]-2-oxo-3-pyrrolidinecarboxylic acid

To a solution of ethyl (3R,4R)-4-[1-methyl-5-(trifluoromethyl)-1H-pyrazol-3-yl]-2-oxo-3-pyrrolidinecarboxylate (i.e. the product obtained in Step G, 20 g, 65.5 mmol) in a mixture of methanol and tetrahydrofuran (1:1, 150 mL) was added lithium hydroxide (3.73 g, 98.3 mmol) in 50 mL of water at 0° C. The reaction mass was then stirred at ambient temperature for 2 h, then diluted with water and washed with methyl tert-butyl ether. The aqueous layer was acidified with aqueous hydrochloric acid (1.5 N) and extracted with ethyl acetate. the ethyl acetate layer was washed with saturated brine solution, dried over sodium sulfate and concentrated under reduced pressure to provide the title compound as a pale yellow liquid 14 g (77%).

¹H NMR: (400 MHz, CDCl₃) δ 6.59 (s, 1H), 4.09 (q, 1H), 3.94 (s, 3H), 3.85-3.77 (m, 1H), 3.72 (d, J=10.0 Hz, 1H), 3.66-3.58 (m, 1H). LC-MS (ESI) m/z 278 (M+H)⁺.

### Step I: Preparation of (3R,4R)-1-methyl-4-[1-methyl-5-(trifluoromethyl)-1H-pyrazol-3-yl]-2-oxo-3-pyrrolidinecarboxylic acid

A solution of (3R,4R)-4-[1-methyl-5-(trifluoromethyl)-1H-pyrazol-3-yl]-2-oxo-3-pyrrolidinecarboxylic acid (i.e. the product obtained in Step H, 14 g, 50.5 mmol) in dry tetrahydrofuran (75 mL) was added to a solution of potassium tert butoxide (1 M solution in tetrahydrofuran, 126 mL) in tetrahydrofuran (75 mL) at 0° C. Iodomethane (7.2 g 101 mmol) was added dropwise at 0° C. The reaction mixture was stirred at ambient temperature for 2 h, then diluted with water, acidified with aqueous hydrochloric acid (1.5 N) and extracted with ethyl acetate. The organic layer was washed with saturated brine solution, dried over sodium sulfate and concentrated under reduced pressure to provide 10 g of the title compound as pale yellow liquid (68%).

¹H NMR: (400 MHz, CDCl₃) δ 6.68 (s, 1H), 3.97 (q, 1H), 3.94 (s, 3H), 3.76-3.68 (m, 3H), 2.99 (s, 3H). LC-MS (ESI) m/z 292 (M+H)⁺.

### Step J: Preparation of (3S,4R)—N-(2,3-difluorophenyl)-1-methyl-4-[1-methyl-5-(trifluoromethyl)-1H-pyrazol-3-yl]-2-oxo-3-pyrrolidinecarboxamide

To a solution of (3R,4R)-1-methyl-4-[1-methyl-5-(trifluoromethyl)-1H-pyrazol-3-yl]-2-oxo-3-pyrrolidinecarboxylic acid (10 g, 34.3 mmol) in ethyl acetate (100 mL) was added triethylamine (10.41 g, 103 mmol) at ambient temperature. T3P (50% solution in ethyl acetate, 32.7 g, 51.5 mmol) was added dropwise at 0° C. The reaction mass was stirred at ambient temperature for 12 h, then washed with water, saturated brine solution, dried over sodium sulfate and concentrated under reduced pressure. The residue obtained was purified by column chromatography eluting with 50% ethyl acetate/petroleum ether to provide the title compound as light pink solid (10 g, 68%).

¹H NMR: (400 MHz, CDCl₃) δ 10.16 (br s, 1H), 8.08-8.01 (m, 1H), 7.02 (ddt, J=2.1, 5.9, 8.3 Hz, 1H), 6.93-6.84 (m, 1H), 6.69 (s, 1H), 4.09 (q, 1H), 3.94 (s, 3H), 3.78 (d, J=9.5 Hz, 1H), 3.76-3.65 (m, 2H), 2.98 (s, 3H). LC-MS (ESI) m/z 403 (M+H)⁺.

### Synthesis Example 2

### Alternate Preparation of (3S,4R)—N-(2,3-difluorophenyl)-1-methyl-4-[1-methyl-5-(trifluoromethyl)-1H-pyrazol-3-yl]-2-oxo-3-pyrrolidinecarboxamide (Compound 13)

### Step A: Preparation of 1-methyl-5-(trifluoromethyl)-1H-pyrazole-3-carboxaldehyde

A stirred solution of 1-methyl-5-(trifluoromethyl)-1H-pyrazole-3-methanol (6.7 g) in dichloromethane (60 mL) was cooled with an ice bath. Pyridinium chlorochromate (9.6 g) was added in portions over 5 minutes. Celite® diatomaceous earth filter aid (15 g) was added and the reaction was stirred at ambient temperature for 2.5 h. The reaction was filtered through a plug of Celite® diatomaceous earth filter aid then concentrated under reduced pressure at 16° C. The resulting black oil was taken up in diethyl ether and passed through a plug of silica, then concentrated under reduced pressure at 16° C. to afford the title compound as a clear oil (4.8 g).

¹H NMR (500 MHz, CDCl₃) δ 9.95 (s, 1H), 7.14 (s, 1H), 4.11 (m, 3H).

### Alternate Preparation of 1-methyl-5-(trifluoromethyl)-1H-pyrazole-3-carboxaldehyde

### Step A1: Preparation of 5,5,5-trifluoro-4-hydroxy-1,1-dimethoxy-3-penten-2-one

A mixture of methylglyoxal 1,1-dimethyl acetal (17.7 g) and ethyl trifluoroacetate (27 g) in diethyl ether (90 mL) was added dropwise over 30 min to a stirred solution of sodium methoxide (30 wt % in methanol, 40 g) in diethyl ether (210 mL) under nitrogen at −5° C. The reaction was stirred at a temperature between −5° C. and 4° C. for 2 h, then was poured into a stirring slurry of concentrated hydrochloric acid (30 mL) and ice chips (150 g). The layers were separated and the aqueous phase was extracted with diethyl ether and methyl tert-butyl ether, then the combined organic extracts were washed with saturated aqueous ammonium chloride solution, dried over sodium sulfate and concentrated under reduced pressure to afford the title compound as a light orange oil (30.5 g) which was used in the next step without further purification.

¹H NMR (500 MHz, CDCl₃, enol tautomer) δ 6.33 (s, 1H), 4.82 (s, 1H), 3.43 (s, 6H).

### Step A2: Preparation of 3-(dimethoxymethyl)-4,5-dihydro-1-methyl-5-(trifluoromethyl)-1H-pyrazol-5-ol

A solution of methyl hydrazine (6.9 g) in methanol (75 mL) was added dropwise over 15 min to a stirred solution of 5,5,5-trifluoro-4-hydroxy-1,1-dimethoxy-3-penten-2-one (i.e. the product of Step A, 30.5 g) in methanol (150 mL) under nitrogen at −5° C. The reaction mixture was stirred at a temperature between −5° C. and 4° C. for 1 h, then was concentrated under reduced pressure to afford an orange oil (34 g). The oil was chromatographed on silica gel, eluting with 0-30% methyl tert-butyl ether in dichloromethane, to provide the title compound as a light yellow oil (23.7 g).

¹H NMR (500 MHz, CDCl₃) δ 4.92 (s, 1H), 3.43 (s, 3H), 3.37 (s, 3H), 3.25-3.21 (m, 1H), 2.99 (s, 3H), 2.95-2.92 (m, 1H).

### Step A3: Preparation of 1-methyl-5-(trifluoromethyl)-1H-pyrazole-3-carboxaldehyde

Concentrated hydrochloric acid (100 mL) was added dropwise over 10 min to a stirred solution to a solution of 3-(dimethoxymethyl)-4,5-dihydro-1-methyl-5-(trifluoromethyl)-1H-pyrazol-5-ol (i.e. the product of Step A2, 31.1 g) in diethyl ether (300 mL) at −36° C. The reaction mixture was then allowed to warm to room temperature and was stirred vigorously for 2 h. The layers were then separated and the aqueous phase was extracted twice with diethyl ether. The combined organic extracts were washed with saturated aqueous ammonium chloride solution (1×), dried over sodium sulfate and concentrated under reduced pressure below 25° C. to afford the title compound as a light yellow oil (18.9 g).

¹H NMR (500 MHz, CDCl₃) δ 9.95 (s, 1H), 7.14 (s, 1H), 4.11 (br s, 3H).

### Step B: Preparation of 1-methyl-3-[(1E)-2-nitroethenyl]-5-(trifluoromethyl)-1H-pyrazole

To a stirred solution of 1-methyl-5-(trifluoromethyl)-1H-pyrazole-3-carboxaldehyde (i.e. the product of Step A or A3, 4.8 g) in methanol (50 mL) was added nitromethane (1.5 mL). The mixture was cooled to −5° C. and aqueous sodium hydroxide (50 wt %, 2.3 g) diluted with water (10 mL) was added dropwise over 15 min, maintaining the temperature below 0° C. Stirring was continued for an additional 3 h between 0° C. and 5° C., then the reaction mixture was poured into 1 N aqueous hydrochloric acid (50 mL). The reaction mixture was transferred to a separatory funnel and the aqueous phase was extracted with ethyl acetate (3×50 mL). The combined organic extracts were washed with brine, dried over magnesium sulfate and concentrated under reduced pressure to afford a yellow oil (5.5 g).

The intermediate thus obtained (5.5 g) was taken up in toluene (50 mL). The solution was cooled to −10° C. with a dry ice/acetone bath and methanesulfonyl chloride (2.0 mL) was added via syringe. Triethylamine (7.3 mL) was then added dropwise over 15 min, maintaining the temperature at or below 0° C. The resulting solution was then stirred for 2 h at the same temperature. The reaction mixture was poured into 1 N aqueous hydrochloric acid (60 mL) and transferred to a separatory funnel. The aqueous phase was extracted with ethyl acetate (3×50 mL) then the combined organic extracts were washed with brine, dried over magnesium sulfate and concentrated under reduced pressure. The crude material was chromatographed on silica gel, eluting with a gradient of 0-10% ethyl acetate in hexanes, to afford the title compound as an amber oil (3.2 g).

¹H NMR (500 MHz, CDCl₃) δ 7.90-7.87 (m, 1H), 7.64-7.62 (m, 1H), 6.88 (s, 1H), 4.06 (m, 3H).

### Step C: Preparation of 1,3-diethyl 2-[(1S)-1-[1-methyl-5-(trifluoromethyl)-1H-pyrazol-3-yl]-2-nitroethyl]propanedioate

To a stirred mixture of 1-methyl-3-[(1E)-2-nitroethenyl]-5-(trifluoromethyl)-1H-pyrazole (i.e. the product of Step B, 3.2 g) and diethyl malonate (3.3 mL) in toluene (25 mL) was added Ni(II) bis[(R,R)—N,N′-dibenzylcyclohexane-1,2-diamine]bromide (prepared as described in J. Am. Chem. Soc. 2005, 127, 9958-9959; 0.232 g). The resulting solution was stirred at ambient temperature for 16 h. The volatiles were then removed under reduced pressure to afford the title compound as an amber oil (5.7 g) which was used without purification.

¹H NMR (500 MHz, CDCl₃) δ 6.53 (s, 1H), 5.03-4.98 (m, 1H), 4.90-4.86 (m, 1H), 4.37-4.33 (m, 1H), 4.25-4.14 (m, 4H), 3.90-3.88 (m, 4H), 1.28-1.19 (m, 6H).

### Step D: Preparation of ethyl (3R,4R)-4-[1-methyl-5-(trifluoromethyl)-1H-pyrazol-3-yl]-2-oxo-3-pyrrolidinecarboxylate

A stirred mixture of 1,3-diethyl 2-[(1S)-1-[1-methyl-5-(trifluoromethyl)-1H-pyrazol-3-yl]-2-nitroethyl]propanedioate (i.e. the product of Step C, 5.7 g), nickel(II) chloride hexahydrate (3.55 g) and ethanol (60 mL) was cooled in an ice bath and treated with sodium borohydride (1.7 g) portionwise over 10 min. The resulting mixture was stirred at ambient temperature for 18 h. Saturated aqueous ammonium chloride solution (100 mL) and ethyl acetate (100 mL) were then added and the mixture was stirred for 2 h. The layers were separated and the aqueous layer was extracted with ethyl acetate (2×100 mL). The combined organic extracts were washed with saturated ammonium chloride solution (100 mL) and brine (100 mL), dried over magnesium sulfate and concentrated under reduced pressure to afford the title compound as a viscous green tinted oil (5.2 g) which was used without purification.

¹H NMR (500 MHz, CDCl₃) δ 6.67 (br s, 1H), 6.47 (s, 1H), 4.30-4.25 (m, 2H), 4.16-4.10 (m, 1H), 3.94-3.93 (m, 3H), 3.81-3.76 (m, 1H), 3.63-3.61 (m, 1H), 3.54-3.50 (m, 1H), 1.33-1.30 (m, 3H).

### Step E: Preparation of (3R,4R)-4-[1-methyl-5-(trifluoromethyl)-1H-pyrazol-3-yl]-2-oxo-3-pyrrolidinecarboxylic acid

A mixture of ethyl (3R,4R)-4-[1-methyl-5-(trifluoromethyl)-1H-pyrazol-3-yl]-2-oxo-3-pyrrolidinecarboxylate (i.e. the product of Step D, 5.2 g) and aqueous sodium hydroxide (50 wt %, 4.1 g) in ethanol (50 mL) was stirred at ambient temperature for 3 h. The reaction mixture was then diluted with water (50 mL) and washed with diethyl ether (2×50 mL). The aqueous phase was acidified with concentrated hydrochloric acid to pH 2 and extracted with ethyl acetate (3×50 mL). The combined ethyl acetate extracts were washed with brine, dried over magnesium sulfate and concentrated under reduced pressure to afford the title compound as a white solid (3.5 g) which was used without further purification.

¹H NMR (500 MHz, CDCl₃) δ 6.66 (s, 1H), 6.34 (br s, 1H), 4.08-4.03 (m, 1H), 3.94 (m, 3H), 3.82-3.78 (m, 1H), 3.72-3.67 (m, 2H).

### Step F: Preparation of (3R,4R)-1-methyl-4-[1-methyl-5-(trifluoromethyl)-1H-pyrazol-3-yl]-2-oxo-3-pyrrolidinecarboxylic acid

A solution of (3R,4R)-4-[1-methyl-5-(trifluoromethyl)-1H-pyrazol-3-yl]-2-oxo-3-pyrrolidinecarboxylic acid (i.e. the product of Step E, 3.5 g) in tetrahydrofuran (10 mL) was added dropwise to an ice bath cooled suspension of potassium tert-butoxide (1 M in tetrahydrofuran, 30.3 mL), maintaining the temperature below 5° C. during addition. The resulting thick solution was stirred at 0° C. for 10 min. Iodomethane (1.34 mL) was added via syringe and the reaction was stirred at ambient temperature for an additional 4 h. The solvent was removed under reduced pressure and the resulting solid was taken up with water (50 mL) and saturated aqueous sodium bicarbonate solution (30 mL) and extracted with diethyl ether (2×50 mL). The aqueous phase was acidified with concentrated hydrochloric acid to pH 2 and extracted with ethyl acetate (3×50 mL). The combined ethyl acetate extracts were washed with brine, dried over magnesium sulfate and concentrated under reduced pressure to afford the title compound as an amber oil (3.5 g).

¹H NMR (500 MHz, CDCl₃) δ 6.68 (s, 1H), 3.98-3.88 (m, 4H), 3.77-3.67 (m, 3H), 2.99 (m, 3H).

### Step G: Preparation of (3S,4R)—N-(2,3-difluorophenyl)-1-methyl-4-[1-methyl-5-(trifluoromethyl)-1H-pyrazol-3-yl]-2-oxo-3-pyrrolidinecarboxamide

A mixture of (3R,4R)-1-methyl-4-[1-methyl-5-(trifluoromethyl)-1H-pyrazol-3-yl]-2-oxo-3-pyrrolidinecarboxylic acid (i.e. the product of Step F, 3.5 g), triethylamine (5 mL) and 2,3-difluoroaniline (1.3 mL) in dichloromethane (40 mL) was cooled with an ice bath and then treated with propylphosphonic anhydride (50 wt % in ethyl acetate, 13.0 g). The resulting mixture was stirred at ambient temperature for 18 h then concentrated under reduced pressure. The crude material was chromatographed on silica gel, eluting with a gradient of 0-50% ethyl acetate in hexanes to provide an oily solid (2.3 g). This material was then triturated with hot hexanes to afford the tittle compound as a white solid (2.1 g).

¹H NMR (500 MHz, CDCl₃) δ 10.16 (br s, 1H), 8.06-8.03 (m, 1H), 7.03-7.00 (m, 1H), 6.91-6.86 (m, 1H), 6.69 (s, 1H), 4.12-4.06 (m, 1H), 3.94 (m, 3H), 3.79-3.66 (m, 3H), 2.98 (m, 3H).

By the procedures described herein together with methods known in the art, the following compounds of Tables 1 to 16 can be prepared. The following abbreviations are used in the Tables which follow: i means iso, Me means methyl, Et means ethyl, Pr means propyl, i-Pr means isopropyl, Ph means phenyl, OMe means methoxy, OEt means ethoxy, SMe means methylthio, S(O)Me means methylsulfinyl, and S(O)₂Me means methylsulfonyl.

TABLE 1

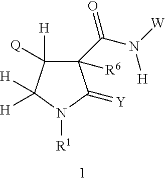

R¹ is Me, R⁶ is H, W is Ph(2-F); Y is O; and Q is;

| | |
|---|---|
| pyrazol-3-yl(1-Me, 5-CF₃) | pyrazol-3-yl(1-Me, 5-I) |
| pyrazol-3-yl 1-Et, 5-CF₃) | pyrazol-3-yl(1-Et, 5-Cl) |
| pyrazol-3-yl(1-i-Pr, 5-CF₃) | pyrazol-3-yl(1-CH₂CF₃, 5-Cl) |
| pyrazol-3-yl(1-CH₂CF₃, 5-CF₃) | pyrazol-3-yl(1-Me, 4,5-di-Cl) |
| pyrazol-3-yl(1-CHF₂, 5-CF₃) | pyrazol-4-yl(1-Me, 5-CF₃) |
| pyrazol-3-yl(1-Me, 5-CF₂CF₃) | pyrazol-4-yl(1,5-dimethyl) |
| pyrazol-3-yl(1-Me, 5-CH₂CF₃) | pyrazol-4-yl(1-Me, 5-OCF₂H) |
| pyrazol-3-yl(1-Me, 4-Cl, 5-CF₃) | pyrazol-4-yl(1,5-dimethyl, 3-Cl) |
| pyrazol-3-yl(1-Me, 4-Br, 5-CF₃) | indazol-3-yl(1-Me) |
| pyrazol-3-yl(1-Me, 4-Me, 5-CF₃) | indazol-3-yl(1-Et) |
| pyrazol-3-yl(1-Me, 5-OCF₂H) | indazol-3-yl(1-i-Pr) |
| pyrazol-3-yl(1-Me, 5-OCF₃) | indazol-3-yl(1-CF₂H) |
| pyrazol-3-yl(1-Me, 5-OCF₂CF₂H) | indazol-3-yl(1-CH₂CF₃) |
| pyrazol-3-yl(1-Me, 5-OCH₂CF₃) | indazol-3-yl(1-Me, 4-F) |
| pyrazol-3-yl(1-Me, 4-Cl, 5-OCF₂H) | indazol-3-yl(1-Me, 4-Cl) |
| pyrazol-3-yl(1-Me, 4-Br, 5-OCF₂H) | indazol-3-yl(1-Me, 4-Br) |
| pyrazol-3-yl(1,5-dimethyl) | indazol-3-yl(1-Me, 5-F) |
| pyrazol-3-yl (1, 5-dimethyl-4-Cl) | indazol-3-yl(1-Me, 5-Cl) |
| pyrazol-3-yl(1-Me, 5-Et) | indazol-3-yl(1-Me, 6-F) |
| pyrazol-3-yl(1-Me, 5-i-Pr) | indazol-3-yl(1-Me, 6-Cl) |
| pyrazol-3-yl(1-Me, 5-OMe) | indazol-3-yl(1-Me, 7-F) |
| pyrazol-3-yl(1-Me, 4-Cl, 5-OMe) | indazol-3-yl(1-Me, 7-Cl) |
| pyrazol-3-yl(1-Me, 5-OEt) | indazol-3-yl(1-Me, 4,7-di-Fl) |
| pyrazol-3-yl(1-Me, 5-O-i-Pr) | indazol-3-yl(1-Me, 4,7-di-Cl |
| pyrazol-3-yl(1-Me, 5-Cl) | indazol-3-yl(1-Me, 4,6-di-F) |
| pyrazol-3-yl(1-Me, 5-F) | indazol-3-yl(1-Me, 5,6-di-Cl) |
| pyrazol-3-yl(1-Me, 5-Br) | |

Table 2 is constructed in the same manner as Table 1 except that the Row Heading “R¹ is Me, R⁶ is H, Y is O, W is Ph(2-F); and Q is” is replaced with the Row Heading listed for Table 2 below (i.e. W is Ph(2,3-di-F); and Q is”). Therefore the first entry in Table 2 is a compound of Formula 1 wherein R¹ is Me, R⁶ is H, Y is O, W is Ph(2,3-di-F); Q is pyrazol-3-yl(1-Me, 5-CF₃). Tables 3 through 16 are constructed similarly.

| Table | Row Heading |
|---|---|
| 2 | R¹ is Me, R⁶ is H, Y is O, W is Ph(2,3-di-F); and Q is |
| 3 | R¹ is Me, R⁶ is H, Y is O, W is Ph(2,4-di-F); and Q is |
| 4 | R¹ is Me, R⁶ is H, Y is O, W is Ph(2,3,4-tri-F); and Q is |
| 5 | R¹ is Me, R⁶ is H, Y is O, W is Ph(2-CF₃); and Q is |
| 6 | R¹ is Me, R⁶ is H, Y is O, W is Ph(2-Me); and Q is |
| 7 | R¹ is Me, R⁶ is H, Y is O, W is Ph(2-NO₂); and Q is |
| 8 | R¹ is Me, R⁶ is H, Y is O, W is Ph(2-Cl); and Q is |
| 9 | R¹ is Me, R⁶ is H, Y is O, W is Ph(2-SO₂Me); and Q is |
| 10 | R¹ is Me, R⁶ is H, Y is O, W is Ph(2-F,3-Cl); and Q is |
| 11 | R¹ is Me, R⁶ is H, Y is O, W is Ph(2-SOMe); and Q is |
| 12 | R¹ is Me, R⁶ is H, Y is O, W is Ph(2-SMe); and Q is |
| 13 | R¹ is Me, R⁶ is H, Y is O, W is Ph(2-Me,3-F); and Q is |
| 14 | R¹ is Me, R⁶ is H, Y is O, W is 3-Pyridinyl(2,6-di-F); and Q is |
| 15 | R¹ is Me, R⁶ is H, Y is O, W is 3-Pyridinyl(2-F); and Q is |
| 16 | R¹ is Me, R⁶ is H, Y is O, W is 2-Pyridinyl(6-F); and Q is |

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill).

Granules and pellets can be prepared by spraying the active material onto preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 13 | 98.5% |
| silica aerogel | 0.5% |
| synthetic amorphous fine silica | 1.0% |

### Example B

Wettable Powder

| | |
|---|---|
| Compound 13 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 13 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 13 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 13 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 13 | 5.0% |
| polyvinylpyrrolidone-vinyl acetate copolymer | 30.0% |
| alkylpolyglycoside | 30.0% |
| glyceryl monooleate | 15.0% |
| Water | 20.0% |

### Example G

Suspension Concentrate

| | |
|---|---|
| Compound 13 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| l,2-benzisothiazolin-3-one | 0.1% |
| Water | 53.7% |

### Example H

Emulsion in Water

| | |
|---|---|
| Compound 13 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| l,2-benzisothiazolin-3-one | 0.1% |
| aromatic petroleum based hydrocarbon | 20.0 |
| Water | 58.7% |

### Example I

Oil Dispersion

| | |
|---|---|
| Compound 13 | 25% |
| polyoxyethylene sorbitol hexaoleate | 15% |
| organically modified bentonite clay | 2.5% |
| fatty acid methyl ester | 57.5% |

The present disclosure also includes Examples A through I above except “Compound 13” is replaced with “Compund 1”, “Compound 2”, “Compound 3”, “Compound 4”, “Compound 5”, “Compound 6”, “Compound 7”, “Compound 8”, “Compound 9”, “Compound 10”, “Compound 11”, “Compound 12”, “Compound 14”, “Compound 15”, “Compound 16”, “Compound 17”, “Compound 18”, “Compound 19”, “Compound 20”, “Compound 21”, “Compound 22”, “Compound 23”, “Compound 24”, “Compound 25”, “Compound 26”, “Compound 27”, “Compound 28”, “Compound 29”, “Compound 30”, “Compound 31” and “Compound 32” above as described in Index Table A.

Test results indicate that the compounds of the present invention are highly active preemergent and/or postemergent herbicides and/or plant growth regulants. The compounds of the invention generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.001 to 20 kg/ha with a preferred range of about 0.004 to 1 kg/ha. One skilled in the art can easily determine the herbicidally effective amount necessary for the desired level of weed control.

In one common embodiment, a compound of the invention is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Although most typically, compounds of the invention are used to control undesired vegetation, contact of desired vegetation in the treated locus with compounds of the invention may result in super-additive or synergistic effects with genetic traits in the desired vegetation, including traits incorporated through genetic modification. For example, resistance to phytophagous insect pests or plant diseases, tolerance to biotic/abiotic stresses or storage stability may be greater than expected from the genetic traits in the desired vegetation.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its esters (e.g., methyl, ethyl) and salts (e.g., sodium, potassium), aminopyralid, amitrole, ammonium sulfamate, anilofos, asulam, atrazine, azimsulfuron, beflubutamid, beflebutamid-M, benazolin, benazolin-ethyl, bencarbazone, benfluralin, benfuresate, bensulfuron-methyl, bensulide, bentazone, benzobicyclon, benzofenap, bicyclopyrone, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyrimorate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenquinotrione, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, fluthiacet-methyl, fomesafen, foramsulfuron, fosamine-ammonium, glufosinate, glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, imazamethabenz-methyl, imazamox, imazapic, imazapyr, imazaquin, imazaquin-ammonium, imazethapyr, imazethapyr-ammonium, imazosulfuron, indanofan, indaziflam, iofensulfuron, iodosulfuron-methyl, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tritosulfuron, vernolate, 3-(2-chloro-3,6-difluorophenyl)-4-hydroxy-1-methyl-1,5-naphthyridin-2(1H)-one, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, methyl 4-amino-3-chloro-6-(4-chloro-2-fluoro-3-methoxyphenyl)-5-fluoro-2-pyridinecarboxylate, 2-methyl-3-(methylsulfonyl)-N-(1-methyl-1H-tetrazol-5-yl)-4-(trifluoromethyl)benzamide and 2-methyl-N-(4-methyl-1,2,5-oxadiazol-3-yl)-3-(methylsulfinyl)-4-(trifluoromethyl)benzamide. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the mixing partners are typically used in the amounts similar to amounts customary when the mixture partners are used alone. More particularly in mixtures, active ingredients are often applied at an application rate between one-half and the full application rate specified on product labels for use of active ingredient alone. These amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the invention with at least one other herbicidal active ingredient. Of particular note is such a combination where the other herbicidal active ingredient has different site of action from the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners such as allidochlor, benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone naphthalic anhydride (1,8-naphthalic anhydride), oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, and 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of the invention cans also be mixed with: (1) polynucleotides including but not limited to DNA, RNA, and/or chemically modified nucleotides influencing the amount of a particular target through down regulation, interference, suppression or silencing of the genetically derived transcript that render a herbicidal effect; or (2) polynucleotides including but not limited to DNA, RNA, and/or chemically modified nucleotides influencing the amount of a particular target through down regulation, interference, suppression or silencing of the genetically derived transcript that render a safening effect.

Of note is a composition comprising a compound of the invention (in a herbicidally effective amount), at least one additional active ingredient selected from the group consisting of other herbicides and herbicide safeners (in an effective amount), and at least one component selected from the group consisting of surfactants, solid diluents and liquid diluents.

Preferred for better control of undesired vegetation (e.g., lower use rate such as from synergism, broader spectrum of weeds controlled, or enhanced crop safety) or for preventing the development of resistant weeds are mixtures of a compound of this invention with a herbicide selected from the group consisting of atrazine, azimsulfuron, beflubutamid, S-beflubutamide, benzisothiazolinone, bixlozone, carfentrazone-ethyl, chlorimuron-ethyl, chlorsulfuron-methyl, clomazone, clopyralid potassium, cloransulam-methyl, 2-[(2,5-dichlorophenyl)methyl]-4,4-dimethyl-3-isoxazolidinone, ethametsulfuron-methyl, flumetsulam, 4-(4-fluorophenyl)-6-[(2-hydroxy-6-oxo-1-cyclohexen-1-yl)carbonyl]-2-methyl-1,2,4-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, S-metolachlor, sulfentrazone, thifensulfuron-methyl, triflusulfuron-methyl and tribenuron-methyl.

Table A1 lists specific combinations of a Component (a) with Component (b) illustrative of the mixtures, compositions and methods of the present invention. Compound 13 in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 13 in Index Table A) with 2,4-D is typically applied in a weight ratio between 1:192-6:1. The remaining lines of Table A1 are to be construed similarly.

TABLE A1

| Component (a) | | Typical | More Typical | Most Typical |
|---|---|---|---|---|
| (Compound #) | Component (b) | Weight Ratio | Weight Ratio | Weight Ratio |
| 13 | 2,4-D | 1:192-6:1 | 1:64-2:1 | 1:24-1:3 |
| 13 | Acetochlor | 1:768-2:1 | 1:256-1:2 | 1:96-1:11 |
| 13 | Acifluorfen | 1:96-12:1 | 1:32-4:1 | 1:12-1:2 |
| 13 | Aclonifen | 1:857-2:1 | 1:285-1:3 | 1:107-1:12 |
| 13 | Alachlor | 1:768-2:1 | 1:256-1:2 | 1:96-1:11 |
| 13 | Ametryn | 1:384-3:1 | 1:128-1:1 | 1:48-1:6 |
| 13 | Amicarbazone | 1:192-6:1 | 1:64-2:1 | 1:24-1:3 |
| 13 | Amidosulfuron | 1:6-168:1 | 1:2-56:1 | 1:1-11:1 |
| 13 | Aminocyclopyrachlor | 1:48-24:1 | 1:16-8:1 | 1:6-2:1 |
| 13 | Aminopyralid | 1:20-56:1 | 1:6-19:1 | 1:2-4:1 |
| 13 | Amitrole | 1:768-2:1 | 1:256-1:2 | 1:96-1:11 |
| 13 | Anilofos | 1:96-12:1 | 1:32-4:1 | 1:12-1:2 |
| 13 | Asulam | 1:960-2:1 | 1:320-1:3 | 1:120-1:14 |
| 13 | Atrazine | 1:192-6:1 | 1:64-2:1 | 1:24-1:3 |
| 13 | Azimsulfuron | 1:6-168:1 | 1:2-56:1 | 1:1-11:1 |
| 13 | Beflubutamid | 1:342-4:1 | 1:114-2:1 | 1:42-1:5 |
| 13 | Beflubutamid-M | 1:171-4:0.5 | 1:62-2:0.5 | 1:21-1: |
| 13 | Benfuresate | 1:617-2:1 | 1:205-1:2 | 1:77-1:9 |
| 13 | Bensulfuron-methyl | 1:25-45:1 | 1:8-15:1 | 1:3-3:1 |
| 13 | Bentazone | 1:192-6:1 | 1:64-2:1 | 1:24-1:3 |
| 13 | Benzobicyclon | 1:85-14:1 | 1:28-5:1 | 1:10-1:2 |
| 13 | Benzofenap | 1:257-5:1 | 1:85-2:1 | 1:32-1:4 |
| 13 | Bicyclopyrone | 1:42-27:1 | 1:14-9:1 | 1:5-2:1 |
| 13 | Bifenox | 1:257-5:1 | 1:85-2:1 | 1:32-1:4 |
| 13 | Bispyribac-sodium | 1:10-112:1 | 1:3-38:1 | 1:1-7:1 |
| 13 | Bixlozone | 1:384-3:1 | 1:128-1:1 | 1:48-1:6 |
| 13 | Bromacil | 1:384-3:1 | 1:128-1:1 | 1:48-1:6 |
| 13 | Bromobutide | 1:384-3:1 | 1:128-1:1 | 1:48-1:6 |
| 13 | Bromoxynil | 1:96-12:1 | 1:32-4:1 | 1:12-1:2 |
| 13 | Butachlor | 1:768-2:1 | 1:256-1:2 | 1:96-1:11 |
| 13 | Butafenacil | 1:42-27:1 | 1:14-9:1 | 1:5-2:1 |
| 13 | Butylate | 1:1542-1:2 | 1:514-1:5 | 1:192-1:22 |
| 13 | Carfenstrole | 1:192-6:1 | 1:64-2:1 | 1:24-1:3 |
| 13 | Carfentrazone-ethyl | 1:128-9:1 | 1:42-3:1 | 1:16-1:2 |
| 13 | Chlorimuron-ethyl | 1:8-135:1 | 1:2-45:1 | 1:1-9:1 |
| 13 | Chlorotoluron | 1:768-2:1 | 1:256-1:2 | 1:96-1:11 |
| 13 | Chlorsulfuron | 1:6-168:1 | 1:2-56:1 | 1:1-11:1 |
| 13 | Cincosulfuron | 1:17-68:1 | 1:5-23:1 | 1:2-5:1 |
| 13 | Cinidon-ethyl | 1:384-3:1 | 1:128-1:1 | 1:48-1:6 |
| 13 | Cinmethylin | 1:34-34:1 | 1:11-12:1 | 1:4-3:1 |
| 13 | Clacyfos | 1:34-34:1 | 1:11-12:1 | 1:4-3:1 |
| 13 | Clethodim | 1:48-24:1 | 1:16-8:1 | 1:6-2:1 |
| 13 | Clodinafop-propargyl | 1:20-56:1 | 1:6-19:1 | 1:2-4:1 |
| 13 | Clomazone | 1:384-3:1 | 1:128-1:1 | 1:48-1:6 |
| 13 | Clomeprop | 1:171-7:1 | 1:57-3:1 | 1:21-1:3 |
| 13 | Clopyralid | 1:192-6:1 | 1:64-2:1 | 1:24-1:3 |
| 13 | Cloransulam-methyl | 1:12-96:1 | 1:4-32:1 | 1:1-6:1 |
| 13 | Cumyluron | 1:384-3:1 | 1:128-1:1 | 1:48-1:6 |
| 13 | Cyanazine | 1:384-3:1 | 1:128-1:1 | 1:48-1:6 |
| 13 | Cyclopyrimorate | 1:17-68:1 | 1:5-23:1 | 1:2-5:1 |
| 13 | Cyclosulfamuron | 1:17-68:1 | 1:5-23:1 | 1:2-5:1 |
| 13 | Cycloxydim | 1:96-12:1 | 1:32-4:1 | 1:12-1:2 |
| 13 | Cyhalofop | 1:25-45:1 | 1:8-15:1 | 1:3-3:1 |
| 13 | Daimuron | 1:192-6:1 | 1:64-2:1 | 1:24-1:3 |
| 13 | Desmedipham | 1:322-4:1 | 1:107-2:1 | 1:40-1:5 |
| 13 | Dicamba | 1:192-6:1 | 1:64-2:1 | 1:24-1:3 |
| 13 | Dichlobenil | 1:1371-1:2 | 1:457-1:4 | 1:171-1:20 |
| 13 | Dichlorprop | 1:925-2:1 | 1:308-1:3 | 1:115-1:13 |
| 13 | Diclofop-methyl | 1:384-3:1 | 1:128-1:1 | 1:48-1:6 |
| 13 | Diclosulam | 1:10-112:1 | 1:3-38:1 | 1:1-7:1 |
| 13 | Difenzoquat | 1:288-4:1 | 1:96-2:1 | 1:36-1:4 |
| 13 | Diflufenican | 1:857-2:1 | 1:285-1:3 | 1:107-1:12 |
| 13 | Diflufenzopyr | 1:12-96:1 | 1:4-32:1 | 1:1-6:1 |
| 13 | Dimethachlor | 1:768-2:1 | 1:256-1:2 | 1:96-1:11 |
| 13 | Dimethametryn | 1:192-6:1 | 1:64-2:1 | 1:24-1:3 |
| 13 | Dimethenamid-P | 1:384-3:1 | 1:128-1:1 | 1:48-1:6 |
| 13 | Dithiopyr | 1:192-6:1 | 1:64-2:1 | 1:24-1:3 |
| 13 | Diuron | 1:384-3:1 | 1:128-1:1 | 1:48-1:6 |
| 13 | EPTC | 1:768-2:1 | 1:256-1:2 | 1:96-1:11 |
| 13 | Esprocarb | 1:1371-1:2 | 1:457-1:4 | 1:171-1:20 |
| 13 | Ethalfluralin | 1:384-3:1 | 1:128-1:1 | 1:48-1:6 |
| 13 | Ethametsulfuron-methyl | 1:17-68:1 | 1:5-23:1 | 1:2-5:1 |
| 13 | Ethoxyfen | 1:8-135:1 | 1:2-45:1 | 1:1-9:1 |
| 13 | Ethoxysulfuron | 1:20-56:1 | 1:6-19:1 | 1:2-4:1 |
| 13 | Etobenzanid | 1:257-5:1 | 1:85-2:1 | 1:32-1:4 |
| 13 | Fenoxaprop-ethyl | 1:120-10:1 | 1:40-4:1 | 1:15-1:2 |
| 13 | Fenoxasulfone | 1:85-14:1 | 1:28-5:1 | 1:10-1:2 |
| 13 | Fenquinotrione | 1:17-68:1 | 1:5-23:1 | 1:2-5:1 |
| 13 | Fentrazamide | 1:17-68:1 | 1:5-23:1 | 1:2-5:1 |
| 13 | Flazasulfuron | 1:17-68:1 | 1:5-23:1 | 1:2-5:1 |
| 13 | Florasulam | 1:2-420:1 | 1:1-140:1 | 2:1-27:1 |
| 13 | Fluazifop-butyl | 1:192-6:1 | 1:64-2:1 | 1:24-1:3 |
| 13 | Flucarbazone | 1:8-135:1 | 1:2-45:1 | 1:1-9:1 |
| 13 | Flucetosulfuron | 1:8-135:1 | 1:2-45:1 | 1:1-9:1 |
| 13 | Flufenacet | 1:257-5:1 | 1:85-2:1 | 1:32-1:4 |
| 13 | Flumetsulam | 1:24-48:1 | 1:8-16:1 | 1:3-3:1 |
| 13 | Flumiclorac-pentyl | 1:10-112:1 | 1:3-38:1 | 1:1-7:1 |
| 13 | Flumioxazin | 1:25-45:1 | 1:8-15:1 | 1:3-3:1 |
| 13 | Fluometuron | 1:384-3:1 | 1:128-1:1 | 1:48-1:6 |
| 13 | Flupyrsulfuron-methyl | 1:3-336:1 | 1:1-112:1 | 2:1-21:1 |
| 13 | Fluridone | 1:384-3:1 | 1:128-1:1 | 1:48-1:6 |
| 13 | Fluroxypyr | 1:96-12:1 | 1:32-4:1 | 1:12-1:2 |
| 13 | Flurtamone | 1:857-2:1 | 1:285-1:3 | 1:107-1:12 |
| 13 | Fluthiacet-methyl | 1:48-42:1 | 1:16-14:1 | 1:3-3:1 |
| 13 | Fomesafen | 1:96-12:1 | 1:32-4:1 | 1:12-1:2 |
| 13 | Foramsulfuron | 1:13-84:1 | 1:4-28:1 | 1:1-6:1 |
| 13 | Glufosinate | 1:288-4:1 | 1:96-2:1 | 1:36-1:4 |
| 13 | Glyphosate | 1:288-4:1 | 1:96-2:1 | 1:36-1:4 |
| 13 | Halosulfuron-methyl | 1:17-68:1 | 1:5-23:1 | 1:2-5:1 |
| 13 | Halauxifen | 1:20-56:1 | 1:6-19:1 | 1:2-4:1 |
| 13 | Halauxifen methyl | 1:20-56:1 | 1:6-19:1 | 1:2-4:1 |
| 13 | Haloxyfop-methyl | 1:34-34:1 | 1:11-12:1 | 1:4-3:1 |
| 13 | Hexazinone | 1:192-6:1 | 1:64-2:1 | 1:24-1:3 |
| 13 | Hydantocidin | 1:1100-16:1 | 1:385-8:1 | 1:144-4:1 |
| 13 | Imazamox | 1:13-84:1 | 1:4-28:1 | 1:1-6:1 |
| 13 | Imazapic | 1:20-56:1 | 1:6-19:1 | 1:2-4:1 |
| 13 | Imazapyr | 1:85-14:1 | 1:28-5:1 | 1:10-1:2 |
| 13 | Imazaquin | 1:34-34:1 | 1:11-12:1 | 1:4-3:1 |
| 13 | Imazethabenz-methyl | 1:171-7:1 | 1:57-3:1 | 1:21-1:3 |
| 13 | Imazethapyr | 1:24-48:1 | 1:8-16:1 | 1:3-3:1 |
| 13 | Imazosulfuron | 1:27-42:1 | 1:9-14:1 | 1:3-3:1 |
| 13 | Indanofan | 1:342-4:1 | 1:114-2:1 | 1:42-1:5 |
| 13 | Indaziflam | 1:25-45:1 | 1:8-15:1 | 1:3-3:1 |
| 13 | Iodosulfuron-methyl | 1:3-336:1 | 1:1-112:1 | 2:1-21:1 |
| 13 | Ioxynil | 1:192-6:1 | 1:64-2:1 | 1:24-1:3 |
| 13 | Ipfencarbazone | 1:85-14:1 | 1:28-5:1 | 1:10-1:2 |
| 13 | Isoproturon | 1:384-3:1 | 1:128-1:1 | 1:48-1:6 |
| 13 | Isoxaben | 1:288-4:1 | 1:96-2:1 | 1:36-1:4 |
| 13 | Isoxaflutole | 1:60-20:1 | 1:20-7:1 | 1:7-2:1 |
| 13 | Lactofen | 1:42-27:1 | 1:14-9:1 | 1:5-2:1 |
| 13 | Lenacil | 1:384-3:1 | 1:128-1:1 | 1:48-1:6 |
| 13 | Linuron | 1:384-3:1 | 1:128-1:1 | 1:48-1:6 |
| 13 | MCPA | 1:192-6:1 | 1:64-2:1 | 1:24-1:3 |
| 13 | MCPB | 1:288-4:1 | 1:96-2:1 | 1:36-1:4 |
| 13 | Mecoprop | 1:768-2:1 | 1:256-1:2 | 1:96-1:11 |
| 13 | Mefenacet | 1:384-3:1 | 1:128-1:1 | 1:48-1:6 |
| 13 | Mefluidide | 1:192-6:1 | 1:64-2:1 | 1:24-1:3 |
| 13 | Mesosulfuron-methyl | 1:5-224:1 | 1:1-75:1 | 1:1-14:1 |
| 13 | Mesotrione | 1:42-27:1 | 1:14-9:1 | 1:5-2:1 |
| 13 | Metamifop | 1:42-27:1 | 1:14-9:1 | 1:5-2:1 |
| 13 | Metazachlor | 1:384-3:1 | 1:128-1:1 | 1:48-1:6 |
| 13 | Metazosulfuron | 1:25-45:1 | 1:8-15:1 | 1:3-3:1 |
| 13 | Methabenzthiazuron | 1:768-2:1 | 1:256-1:2 | 1:96-1:11 |
| 13 | Metolachlor | 1:768-2:1 | 1:256-1:2 | 1:96-1:11 |
| 13 | Metosulam | 1:8-135:1 | 1:2-45:1 | 1:1-9:1 |
| 13 | Metribuzin | 1:192-6:1 | 1:64-2:1 | 1:24-1:3 |
| 13 | Metsulfuron-methyl | 1:2-560:1 | 1:1-187:1 | 3:1-35:1 |
| 13 | Molinate | 1:1028-2:1 | 1:342-1:3 | 1:128-1:15 |
| 13 | Napropamide | 1:384-3:1 | 1:128-1:1 | 1:48-1:6 |
| 13 | Napropamide-M | 1:192-6:1 | 1:64-2:1 | 1:24-1:3 |
| 13 | Naptalam | 1:192-6:1 | 1:64-2:1 | 1:24-1:3 |
| 13 | Nicosulfuron | 1:12-96:1 | 1:4-32:1 | 1:1-6:1 |
| 13 | Norflurazon | 1:1152-1:1 | 1:384-1:3 | 1:144-1:16 |
| 13 | Orbencarb | 1:1371-1:2 | 1:457-1:4 | 1:171-1:20 |
| 13 | Ortho sulfamuron | 1:20-56:1 | 1:6-19:1 | 1:2-4:1 |
| 13 | Oryzalin | 1:514-3:1 | 1:171-1:2 | 1:64-1:8 |
| 13 | Oxadiargyl | 1:384-3:1 | 1:128-1:1 | 1:48-1:6 |
| 13 | Oxadiazon | 1:548-3:1 | 1:182-1:2 | 1:68-1:8 |
| 13 | Oxasulfuron | 1:27-42:1 | 1:9-14:1 | 1:3-3:1 |
| 13 | Oxaziclomefone | 1:42-27:1 | 1:14-9:1 | 1:5-2:1 |
| 13 | Oxyfluorfen | 1:384-3:1 | 1:128-1:1 | 1:48-1:6 |
| 13 | Paraquat | 1:192-6:1 | 1:64-2:1 | 1:24-1:3 |
| 13 | Pendimethalin | 1:384-3:1 | 1:128-1:1 | 1:48-1:6 |
| 13 | Penoxsulam | 1:10-112:1 | 1:3-38:1 | 1:1-7:1 |
| 13 | Penthoxamid | 1:384-3:1 | 1:128-1:1 | 1:48-1:6 |
| 13 | Pentoxazone | 1:102-12:1 | 1:34-4:1 | 1:12-1:2 |
| 13 | Phenmedipham | 1:102-12:1 | 1:34-4:1 | 1:12-1:2 |
| 13 | Picloram | 1:96-12:1 | 1:32-4:1 | 1:12-1:2 |
| 13 | Picolinafen | 1:34-34:1 | 1:11-12:1 | 1:4-3:1 |
| 13 | Pinoxaden | 1:25-45:1 | 1:8-15:1 | 1:3-3:1 |
| 13 | Pretilachlor | 1:192-6:1 | 1:64-2:1 | 1:24-1:3 |
| 13 | Primisulfuron-methyl | 1:8-135:1 | 1:2-45:1 | 1:1-9:1 |
| 13 | Prodiamine | 1:384-3:1 | 1:128-1:1 | 1:48-1:6 |
| 13 | Profoxydim | 1:42-27:1 | 1:14-9:1 | 1:5-2:1 |
| 13 | Prometryn | 1:384-3:1 | 1:128-1:1 | 1:48-1:6 |
| 13 | Propachlor | 1:1152-1:1 | 1:384-1:3 | 1:144-1:16 |
| 13 | Propanil | 1:384-3:1 | 1:128-1:1 | 1:48-1:6 |
| 13 | Propaquizafop | 1:48-24:1 | 1:16-8:1 | 1:6-2:1 |
| 13 | Propoxycarbazone | 1:17-68:1 | 1:5-23:1 | 1:2-5:1 |
| 13 | Propyrisulfuron | 1:17-68:1 | 1:5-23:1 | 1:2-5:1 |
| 13 | Propyzamide | 1:384-3:1 | 1:128-1:1 | 1:48-1:6 |
| 13 | Prosulfocarb | 1:1200-1:2 | 1:400-1:4 | 1:150-1:17 |
| 13 | Prosulfuron | 1:6-168:1 | 1:2-56:1 | 1:1-11:1 |
| 13 | Pyraclonil | 1:42-27:1 | 1:14-9:1 | 1:5-2:1 |
| 13 | Pyraflufen-ethyl | 1:5-224:1 | 1:1-75:1 | 1:1-14:1 |
| 13 | Pyrasulfotole | 1:13-84:1 | 1:4-28:1 | 1:1-6:1 |
| 13 | Pyrazolynate | 1:857-2:1 | 1:285-1:3 | 1:107-1:12 |
| 13 | Pyrazosulfuron-ethyl | 1:10-112:1 | 1:3-38:1 | 1:1-7:1 |
| 13 | Pyrazoxyfen | 1:5-224:1 | 1:1-75:1 | 1:1-14:1 |
| 13 | Pyribenzoxim | 1:10-112:1 | 1:3-38:1 | 1:1-7:1 |
| 13 | Pyributicarb | 1:384-3:1 | 1:128-1:1 | 1:48-1:6 |
| 13 | Pyridate | 1:288-4:1 | 1:96-2:1 | 1:36-1:4 |
| 13 | Pyriftalid | 1:10-112:1 | 1:3-38:1 | 1:1-7:1 |
| 13 | Pyriminobac-methyl | 1:20-56:1 | 1:6-19:1 | 1:2-4:1 |
| 13 | Pyrimisulfan | 1:17-68:1 | 1:5-23:1 | 1:2-5:1 |
| 13 | Pyrithiobac | 1:24-48:1 | 1:8-16:1 | 1:3-3:1 |
| 13 | Pyroxasulfone | 1:85-14:1 | 1:28-5:1 | 1:10-1:2 |
| 13 | Pyroxsulam | 1:5-224:1 | 1:1-75:1 | 1:1-14:1 |
| 13 | Quinclorac | 1:192-6:1 | 1:64-2:1 | 1:24-1:3 |
| 13 | Quizalofop-ethyl | 1:42-27:1 | 1:14-9:1 | 1:5-2:1 |
| 13 | Rimsulfuron | 1:13-84:1 | 1:4-28:1 | 1:1-6:1 |
| 13 | Saflufenacil | 1:25-45:1 | 1:8-15:1 | 1:3-3:1 |
| 13 | Sethoxydim | 1:96-12:1 | 1:32-4:1 | 1:12-1:2 |
| 13 | Simazine | 1:384-3:1 | 1:128-1:1 | 1:48-1:6 |
| 13 | Sulcotrione | 1:120-10:1 | 1:40-4:1 | 1:15-1:2 |
| 13 | Sulfentrazone | 1:147-8:1 | 1:49-3:1 | 1:18-1:3 |
| 13 | Sulfometuron-methyl | 1:34-34:1 | 1:11-12:1 | 1:4-3:1 |
| 13 | Sulfosulfuron | 1:8-135:1 | 1:2-45:1 | 1:1-9:1 |
| 13 | Tebuthiuron | 1:384-3:1 | 1:128-1:1 | 1:48-1:6 |
| 13 | Tefuryltrione | 1:42-27:1 | 1:14-9:1 | 1:5-2:1 |
| 13 | Tembotrione | 1:31-37:1 | 1:10-13:1 | 1:3-3:1 |
| 13 | Tepraloxydim | 1:25-45:1 | 1:8-15:1 | 1:3-3:1 |
| 13 | Terbacil | 1:288-4:1 | 1:96-2:1 | 1:36-1:4 |
| 13 | Terbuthylazine | 1:857-2:1 | 1:285-1:3 | 1:107-1:12 |
| 13 | Terbutryn | 1:192-6:1 | 1:64-2:1 | 1:24-1:3 |
| 13 | Thenylchlor | 1:85-14:1 | 1:28-5:1 | 1:10-1:2 |
| 13 | Thiazopyr | 1:384-3:1 | 1:128-1:1 | 1:48-1:6 |
| 13 | Thiencarbazone | 1:3-336:1 | 1:1-112:1 | 2:1-21:1 |
| 13 | Thifensulfuron-methyl | 1:5-224:1 | 1:1-75:1 | 1:1-14:1 |
| 13 | Tiafenacil | 1:17-68:1 | 1:5-23:1 | 1:2-5:1 |
| 13 | Thiobencarb | 1:768-2:1 | 1:256-1:2 | 1:96-1:11 |
| 13 | Tolpyralate | 1:31-37:1 | 1:10-13:1 | 1:3-3:1 |
| 13 | Topramzone | 1:6-168:1 | 1:2-56:1 | 1:1-11:1 |
| 13 | Tralkoxydim | 1:68-17:1 | 1:22-6:1 | 1:8-2:1 |
| 13 | Triafamone | 1:2-420:1 | 1:1-140:1 | 2:1-27:1 |
| 13 | Triallate | 1:768-2:1 | 1:256-1:2 | 1:96-1:11 |
| 13 | Triasulfuron | 1:5-224:1 | 1:1-75:1 | 1:1-14:1 |
| 13 | Triaziflam | 1:171-7:1 | 1:57-3:1 | 1:21-1:3 |
| 13 | Tribenuron-methyl | 1:3-336:1 | 1:1-112:1 | 2:1-21:1 |
| 13 | Triclopyr | 1:192-6:1 | 1:64-2:1 | 1:24-1:3 |
| 13 | Trifloxysulfuron | 1:2-420:1 | 1:1-140:1 | 2:1-27:1 |
| 13 | Trifludimoxazin | 1:25-45:1 | 1:8-15:1 | 1:3-3:1 |
| 13 | Trifluralin | 1:288-4:1 | 1:96-2:1 | 1:36-1:4 |
| 13 | Triflusulfuron-methyl | 1:17-68:1 | 1:5-23:1 | 1:2-5:1 |
| 13 | Tritosulfuron | 1:13-84:1 | 1:4-28:1 | 1:1-6:1 |

Table A2 is constructed the same as Table A1 above except that entries below the “Component (a)” column heading are replaced with the respective Component (a) Column Entry shown below. Compound 1 in the Component (a) column is identified in Index Table A. Thus, for example, in Table A2 the entries below the “Component (a)” column heading all recite “Compound 2” (i.e. Compound 2 identified in Index Table A). and the first line below the column headings in Table A2 specifically discloses a mixture of Compound 2 with 2,4-D. Tables A3 through A31 are constructed similarly.

| Table Number | Component (a) Column Entries |
|---|---|
| A2 | Compound 2 |
| A3 | Compound 3 |
| A4 | Compound 4 |
| A5 | Compound 5 |
| A6 | Compound 6 |
| A7 | Compound 7 |
| A8 | Compound 8 |
| A9 | Compound 9 |
| A10 | Compound 10 |
| A11 | Compound 11 |
| A12 | Compound 12 |
| A13 | Compound 14 |
| A14 | Compound 15 |
| A15 | Compound 16 |
| A16 | Compound 17 |
| A17 | Compound 18 |
| A18 | Compound 19 |
| A19 | Compound 20 |
| A20 | Compound 21 |
| A21 | Compound 22 |
| A22 | Compound 23 |
| A23 | Compound 24 |
| A24 | Compound 25 |
| A25 | Compound 26 |
| A26 | Compound 27 |
| A27 | Compound 28 |
| A28 | Compound 29 |
| A29 | Compound 30 |
| A30 | Compound 31 |
| A31 | Compound 32 |

Preferred for better control of undesired vegetation (e.g., lower use rate such as from synergism, broader spectrum of weeds controlled, or enhanced crop safety) or for preventing the development of resistant weeds are mixtures of a compound of this invention with a herbicide selected from the group consisting of chlorsulfuron, ethametsulfuron, chlorimuron-ethyl, mesotrione, thifensulfuron-methyl, flupyrsulfuron-methyl, tribenuron-methyl, metsulfuron-methyl, triflusulfuron-methyl, pyroxasulfone, pinoxaden, tembotrione, pyroxsulam, metolachlor and S-metolachlor.

The following Tests demonstrate the control efficacy of the compounds of this invention against specific weeds. The weed control afforded by the compounds is not limited, however, to these species. See Index Tables A and B for compound descriptions. The following abbreviations are used in the Index Table which follows: Et is ethyl. (R) or (S) denotes the absolute chirality of the asymmetric carbon center. “rac.” means a racemic mixture. “Stereo (3,4)” describes the stereochemistry at the 3- and 4-positions of the pyrrolidinone ring. The abbreviation “Cmpd. No.” stands for “Compound Number”. The abbreviation “Ex.” stands for “Example” and is followed by a number indicating in which Synthesis Example the compound is prepared. Mass spectra are reported with an estimated precision within ±0.5 Da as the molecular weight of the highest isotopic abundance parent ion (M+1) formed by addition of H⁺ (molecular weight of 1) to the molecule observed by using atmospheric pressure chemical ionization (AP+).

INDEX TABLE A

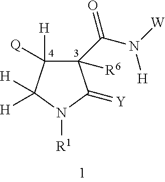

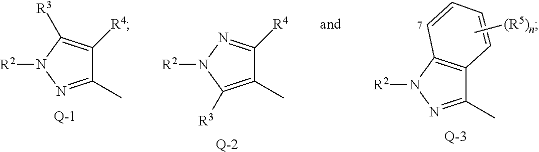

| Cmpd. | | | | | | | | | Stereo | M.P. |
|---|---|---|---|---|---|---|---|---|---|---|
| No. | Q | R¹ | R² | R³ | R⁴ | (R⁵)ₙ | Y | W | (3,4) | (M.S.) |
| 1 | Q-1 | H | CH₃ | t-Bu | H | — | O | 2-pyr(6-F) | S,R | * |
| 2 | Q-3 | CH₃ | CH₃ | — | — | 7-F | O | Ph(2,3-di-F) | S,R | * |
| 3 | Q-1 | CH₃ | CH₃ | CF₃ | H | — | O | Ph(2,3-di-F) | rac | * |
| 4 | Q-3 | CH₃ | Et | — | — | 6-F | O | Ph(2,3-di-F) | S,R | * |
| 5 | Q-3 | CH₃ | CH₃ | — | — | 7-Cl | O | Ph(2,3-di-F) | S,R | * |
| 6 | Q-1 | CH₃ | CH₃ | CF₃ | H | — | O | Ph(2,4-di-F) | rac. | * |
| 7 | Q-3 | CH₃ | Et | — | — | 4-F | O | Ph(2,3-di-F) | S,R | * |
| 8 | Q-3 | CH₃ | Et | — | — | 5-F | O | Ph(2,3-di-F) | S,R | * |
| 9 | Q-1 | CH₃ | CH₃ | CF₃ | H | — | O | Ph(2-F) | rac. | * |
| 10 | Q-3 | CH₃ | Et | — | — | 7-F | O | Ph(2,3-di-F) | rac. | * |
| 11 | Q-3 | CH₃ | Et | — | — | 7-F | O | 3-pyr(2,6-di-F) | S,R | * |
| 12 | Q-1 | CH₃ | CH₃ | CF₃ | H | — | O | Ph(2,3-di-F) | R,R | * |
| 13 | Q-1 | CH₃ | CH₃ | CF₃ | H | — | O | Ph(2,3-di-F) | S,R | (403 (+))** |
| (Ex. 1) | | | | | | | | | | |
| 14 | Q-3 | CH₃ | Et | — | — | 7-F | O | Ph(2,3-di-F) | S,S | * |
| 15 | Q-3 | CH₃ | Et | — | — | 7-F | O | Ph(2,3-di-F) | R,R | * |
| 16 | Q-1 | CH₃ | CH₃ | CF₃ | H | — | O | Ph(3-F,2-OCF₃) | rac. | * |
| 17 | Q-1 | CH₃ | CH₃ | CF₃ | H | — | O | Ph(2,3,4-tri-F) | rac. | * |
| 18 | Q-1 | CH₃ | CH₃ | CF₃ | H | — | O | Ph(3-F,2-OCH₃) | rac. | * |
| 19 | Q-1 | CH₃ | CH₃ | CF₃ | H | — | O | Ph(3-F,2-CF₃) | rac. | * |
| 20 | Q-1 | CH₃ | CH₃ | CF₃ | H | — | O | Ph(2-CH₂CH₃) | rac. | * |
| 21 | Q-3 | CH₃ | CH₃ | — | — | 7-F | O | Ph(2,3-di-F) | rac. | * |
| 22 | Q-1 | CH₃ | CH₃ | CF₃ | H | — | O | 3-pyr(2,6-di-F) | rac. | * |
| 23 | Q-1 | CH₃ | CH₃ | CF₃ | H | — | O | Ph(3-F,2-0CHF₂) | rac. | * |
| 24 | Q-1 | CH₃ | CH₃ | CF₃ | H | — | O | 2-pyr(6-F) | rac. | * |
| 25 | Q-2 | CH₃ | CH₃ | CF₃ | H | — | O | Ph(2,3-di-F) | rac. | * |
| 26 | Q-1 | CH₃ | CH₃ | CF₃ | H | — | O | Ph(4-F,2-CF₂CH₃) | rac. | * |
| 27 | Q-1 | CH₃ | CH₃ | Cl | H | — | O | Ph(2,3-di-F) | rac. | * |
| 28 | Q-1 | CH₃ | CH₃ | Cl | Br | — | O | Ph(2,3-di-F) | rac. | * |
| 29 | Q-1 | CH₃ | CH₃ | Cl | H | — | O | Ph(2,4-di-F) | rac. | * |
| 30 | Q-1 | CH₃ | CH₃ | Cl | Br | — | O | Ph(2,4-di-F) | rac. | * |
| 31 | Q-1 | CH₃ | CH₃ | Cl | H | — | O | Ph(2,3,4-tri-F) | rac. | * |
| 32 | Q-1 | CH₃ | CH₃ | Cl | Br | — | O | Ph(2,3,4-tri-F) | rac. | * |

*See Index Table B for ¹H NMR data.
**See Synthesis Example for ¹H NMR data.

INDEX TABLE B

| Cmpd. No. | ¹H NMR (ppm) |
|---|---|
| 1 | δ 9.81 (br.s, 1H), 8.07 (dd, 1H), 7.75 (q, 1H), 6.63 (dd, 1H), 6.53 (br.s, 1H), 5.98 (s, 1H), |
| | 4.17 (q, 1H), 3.92 (s, 3H), 3.78 (q, 1H), 3.76 (dt, 1H), 3.57 (t, 1H), 1.36 (s, 9H). |
| 2 | δ 9.96-10.06 (m, 1H), 8.02 (ddt, J = 8.34, 6.66, 1.54, 1.54 Hz, 1H), 7.55-7.62 (m, 1H), |
| | 7.03-7.07 (m, 2H), 6.96-7.02 (m, 2H), 6.83-6.91 (m, 2H), 4.56 (td, J = 8.93, 7.83 Hz, 1H), |
| | 4.20 (d, J = 0.98 Hz, 3H), 4.10 (d, J = 8.44 Hz, 1H), 3.73-3.91 (m, 2H), 3.01 (d, J = 0.73 Hz, |
| | 4H). |
| 3 | δ 10.15 (br s, 1H), 8.04 (tdd, J = 1.6, 6.6, 8.3 Hz, 1H), 7.02 (ddt, J = 2.1, 5.9, 8.3 Hz, 1H), |
| | 6.93-6.85 (m, 1H), 6.69 (s, 1H), 4.09 (q, 1H), 3.94 (s, 3H), 3.81-3.65 (m, 3H), 2.98 (d, |
| | 3H) |
| 4 | δ 9.86-10.05 (m, 1H), 7.99-8.05 (m, 1H), 7.82 (dd, J = 9.05, 5.01 Hz, 1H), 6.98-7.04 (m, |
| | 2H), 6.84-6.97 (m, 2H), 4.58 (q, J = 8.60 Hz, 1H), 4.32 (q, J = 7.21 Hz, 2H), 4.05 (d, J = 8.31 |
| | Hz, 1H), 3.81-3.89 (m, 2H), 3.02 (d, J = 0.73 Hz, 3H), 1.48 (t, J = 7.27 Hz, 3H). |
| 5 | δ 9.97 (br s, 1H), 7.98-8.04 (m, 1H), 7.71-7.76 (m, 1H), 7.32-7.40 (m, 2H), 7.04-7.11 |
| | (m, 2H), 6.94-7.04 (m, 2H), 6.81-6.94 (m, 2H), 4.52-4.61 (m, 1H), 4.36 (s, 3H), 4.11 (d, |
| | J = 8.68 Hz, 1H), 3.73-3.91 (m, 3H), 3.01 (d, J = 0.73 Hz, 3H). |
| 6 | δ 9.98 (br s, 1H), 8.22 (dt, J = 6.0, 8.9 Hz, 1H), 6.90-6.80 (m, 2H), 6.69 (s, 1H), 4.09 (q, |
| | 1H), 3.94 (d, 3H), 3.80-3.65 (m, 3H), 2.97 (d, J = 0.7 Hz, 3H). |
| 7 | δ 10.01 (s, 1H), 8.04 (s, 1H), 8.01-7.93 (m, 1H), 7.23 (s, 1H), 7.05-6.96 (m, 1H), 6.93- |
| | 6.83 (m, 1H), 6.76 (dd, J = 0.7, 10.7 Hz, 1H), 4.43 (q, J = 7.3 Hz, 2H), 4.24 (q, 1H), 3.85 |
| | (t, J = 9.8 Hz, 1H), 3.71 (d, J = 8.9 Hz, 1H), 3.53 (dd, J = 7.8, 10.2 Hz, 1H), 3.03 (s, 3H), |
| | 1.53 (t, J = 7.3 Hz, 3H). |
| 8 | δ 10.01 (brs, 1H), 8.08-8.01 (m, 1H), 7.50 (dd, J = 1.9, 8.7 Hz, 1H), 7.31 (d, J = 4.0 Hz, |
| | 1H), 7.17 (td, J = 2.4 Hz, 1H), 7.05-6.97 (m, 1H), 6.92-6.82 (m, 1H), 4.53 (q, J = 8.4 Hz, |
| | 1H), 4.37 (q, J = 7.2 Hz, 2H), 4.07 (d, J = 8.6 Hz, 1H), 3.84 (d, J = 8.6 Hz, 2H), 3.02 (s, |
| | 3H), 1.49 (t, J = 7.3 Hz, 3H). |
| 9 | δ 10.04 (br s, 1H), 8.31-8.25 (m, 1H), 7.13-7.00 (m, 3H), 6.69 (s, 1H), 4.11 (q, 1H), 3.94 |
| | (s, 3H), 3.80-3.65 (m, 3H), 2.98 (d, 3H). |
| 10 | δ 10.05-9.90 (m, 1H), 8.03 (tdd, J = 1.6, 6.6, 8.3 Hz, 1H), 7.64-7.56 (m, 1H), 7.10-6.94 |
| | (m, 3H), 6.93-6.82 (m, 1H), 4.62-1.50 (m, 3H), 4.10 (d, J = 8.6 Hz, 1H), 3.89-3.79 (m, |
| | 2H), 3.02 (d, J = 0.7 Hz, 3H), 1.49 (t, J = 7.0 Hz, 4H). |
| 11 | δ 10.02 (brs, 1H), 8.84-8.74 (m, 1H), 7.48 (dd, J = 1.9, 8.7 Hz, 1H), 7.33 (dd, J = 3.9, 9.0 |
| | Hz, 1H), 7.18 (dt, J = 2.3, 8.9 Hz, 1H), 6.79 (dd, J = 2.9, 8.6 Hz, 1H), 4.49 (q, J = 8.6 Hz, |
| | 1H), 4.38 (q, J = 7.2 Hz, 2H), 4.08 (d, J = 8.9 Hz, 1H), 3.85 (d, J = 8.8 Hz, 2H), 3.02 (s, |
| | 3H), 1.49 (t, J = 7.3 Hz, 3H). |
| 12 | Enantiomer of Cmpd. No. 13. δ 10.15 (br s, 1H), 8.04 (dd, J = 6.6, 8.3 Hz, 1H), 7.06- |
| | 6.99 (m, 1H), 6.89 (br dd, J = 1.1, 8.6 Hz, 1H), 6.69 (s, 1H), 4.09 (q, 1H), 3.94 (s, 3H), |
| | 3.78 (d, J = 9.5 Hz, 1H), 3.76-3.65 (m, 2H), 2.98 (d, 3H). |
| 13 | δ 10.15 (br s, 1H), 8.04 (dd, J = 6.6, 8.3 Hz, 1H), 7.06-6.99 (m, 1H), 6.89 (br dd, J = 1.1, |
| | 8.6 Hz, 1H), 6.69 (s, 1H), 4.09 (q, 1H), 3.94 (s, 3H), 3.78 (d, J = 9.5 Hz, 1H), 3.76-3.65 |
| | (m, 2H), 2.98 (d, 3H). |
| 14 | δ 10.00 (br s, 1H), 8.03 (tdd, J = 1.6, 6.6, 8.3 Hz, 1H), 7.65-7.57 (m, 1H), 7.10-6.96 (m, |
| | 3H), 6.87 (dddd, J = 1.5, 7.3, 8.5, 9.8 Hz, 1H), 4.59-4.51 (m, 3H), 4.10 (d, J = 8.7 Hz, |
| | 1H), 3.91-3.79 (m, 2H), 3.01 (d, J = 0.7 Hz, 3H), 1.49 (t, J = 7.2 Hz, 3H). |
| 15 | δ 10.00 (br s, 1H), 8.08-7.98 (m, 1H), 7.65-7.56 (m, 1H), 7.10-6.95 (m, 3H), 6.94-6.83 |
| | (m, 1H), 4.62-4.51 (m, 3H), 4.10 (d, J = 8.6 Hz, 1H), 3.90-3.79 (m, 2H), 3.02 (d, J = 0.6 |
| | Hz, 3H), 1.49 (t, J = 7.1 Hz, 3H). |
| 16 | δ 10.40 (s, 1H), 8.17 (td, J = 1.5, 8.5 Hz, 1H), 7.26-7.19 (m, 1H), 6.92 (ddd, J = 1.4, 8.4, |
| | 9.7 Hz, 1H), 6.69 (s, 1H), 4.07 (q, J = 9.0 Hz, 1H), 3.94 (s, 3H), 3.77 (d, 1H), 3.74-3.64 |
| | (m, 2H), 2.98 (s, 3H). |
| 17 | δ 10.08 (br s, 1H), 8.01-7.94 (m, 1H), 6.92 (ddt, J = 2.4, 7.7, 9.7 Hz, 1H), 6.68 (s, 1H), |
| | 4.07 (q, 1H), 3.94 (s, 3H), 3.77 (d, 1H), 3.75-3.65 (m, 2H), 2.98 (d, 3H). |
| 18 | δ 10.21 (s, 1H), 8.13 (td, J = 1.3, 8.4 Hz, 1H), 6.96 (dt, J = 5.7, 8.3 Hz, 1H), 6.81 (ddd, J = |
| | 1.5, 8.4, 11.1 Hz, 1H), 6.68 (s, 1H), 4.13 (q, J = 9.0 Hz, 1H), 4.03 (d, J = 1.7 Hz, 3H), |
| | 3.94 (d, 3H), 3.78-3.63 (m, 3H), 2.97 (d, J = 0.7 Hz, 3H). |
| 19 | δ 10.16 (br s, 1H), 7.99 (d, J = 8.3 Hz, 1H), 7.46 (dt, J = 6.0, 8.4 Hz, 1H), 7.00-6.92 (m, |
| | 1H), 6.68 (s, 1H), 4.09 (q, J = 8.9 Hz, 1H), 3.94 (s, 3H), 3.79-3.66 (m, 3H), 2.98 (d, 3H) |
| 20 | δ 9.73 (s, 1H), 8.05 (d, 1H), 7.34-7.27 (m, 1H), 7.22-7.16 (m, 1H), 7.10-7.05 (m, 1H), |
| | 6.72 (s, 1H), 4.17-4.07 (m, 1H), 3.94 (s, 3H), 3.77-3.66 (m, 3H), 2.97 (d, 3H), 2.77-2.65 |
| | (m, 2H), 1.27 (t, 3H). |
| 21 | δ 10.02 (br s, 1H), 8.02 (tdd, J = 1.6, 6.6, 8.3 Hz, 1H), 7.62-7.55 (m, 1H), 7.09-6.96 (m, |
| | 3H), 6.92-6.82 (m, 1H), 4.56 (dt, J = 7.9, 8.9 Hz, 1H), 4.20 (d, J = 0.9 Hz, 3H), 4.10 (d, J = |
| | 8.4 Hz, 1H), 3.89-3.75 (m, 2H), 3.01 (d, J = 0.7 Hz, 3H). |
| 22 | δ 10.17 (br s, 1H), 8.83-8.76 (m, 1H), 6.80 (dd, J = 2.9, 8.6 Hz, 1H), 6.67 (s, 1H), 4.07 |
| | (q, J = 8.9 Hz, 1H), 3.95 (d, 3H), 3.83-3.65 (m, 3H), 2.98 (d, 3H). |
| 23 | δ 10.29 (s, 1H), 8.17 (td, J = 1.3, 8.4 Hz, 1H), 7.17 (dt, J = 5.9, 8.5 Hz, 1H), 6.89 (ddd, J |
| | 1.3, 8.5, 10.0 Hz, 1H), 6.68 (s, 1H), 6.67 (t [large F coupling], 1H), 4.09 (q, J = 9.0 Hz, |
| | 1H), 3.94 (s, 3H), 3.78 (d, J = 9.5 Hz, 1H), 3.75-3.63 (m, 2H), 2.98 (m, 3H). |
| 24 | δ 10.04 (s, 1H), 8.01 (dd, J = 1.8, 7.9 Hz, 1H), 7.75 (q, J = 8.1 Hz, 1H), 6.65 (s, 1H), 6.64 |
| | (dd, 1H), 4.12 (q, J = 9.0 Hz, 1H), 3.94 (s, 3H), 3.77-3.61 (m, 3H), 2.96 (s, 3H). |
| 25 | δ 10.05 (br s, 1H), 8.04-7.97 (m, 1H), 7.46 (s, 1H), 7.01 (ddt, J = 2.1, 5.9, 8.3 Hz, 1H), |
| | 6.93-6.84 (m, 1H), 4.21 (q, J = 8.8 Hz, 1H), 4.00 (s, 3H), 3.75 (t, J = 9.5 Hz, 1H), 3.64 |
| | (d, J = 9.4 Hz, 1H), 3.27 (dd, J = 8.1, 9.9 Hz, 1H), 2.97 (s, 3H). |
| 26 | δ 9.75 (br s, 1H), 8.11 (dd, J = 5.1, 9.0 Hz, 1H), 7.21 (dd, J = 2.9, 9.2 Hz, 1H), 7.13-7.06 |
| | (m, 1H), 6.67 (s, 1H), 4.13 (q, J = 8.9 Hz, 1H), 3.94 (s, 3H), 3.76-3.64 (m, 3H), 2.97 (s, |
| | 3H), 1.98 (t, 3H). |
| 27 | δ 10.14 (s, 1H), 8.09-7.97 (m, 1H), 7.08-6.97 (m, 1H), 6.92-6.82 (m, 1H), 4.10-3.97 (m, |
| | 1H), 3.88-3.75 (m, 1H), 3.80 (s, 3H), 3.74-3.60 (m, 2H), 2.95 (s, 3H). |
| 28 | δ 10.05 (brs, 1H), 8.09-8.01 (m, 1H), 7.04-6.96 (m, 1H), 6.91-6.82 (m, 1H), 4.23-4.10 |
| | (m, 2H), 3.83 (s, 3H), 3.85-3.77 (m, 1H), 3.41-3.34 (m, 1H), 2.96 (s, 3H). |
| 29 | δ 9.96 (brs, 1H), 8.28-8.18 (m, 1H), 6.91-6.77 (m, 2H), 6.27 (s, 1H), 4.05 (q, J = 9.0 Hz, |
| | 1H), 3.83-3.60 (m, 3H), 3.79 (s, 3H), 2.96 (s, 3H). |
| 30 | δ 9.88 (brs, 1H), 8.28-8.17 (m, 1H), 6.91-6.77 (m, 2H), 4.19-4.13 (m, 2H), 3.85-3.75 |
| | (m, 1H), 3.84 (s, 3H), 3.41-3.34 (m, 1H), 2.96 (s, 3H). |
| 31 | δ 10.06 (s, 1H), 8.03-7.93 (m, 1H), 6.98-6.85 (m, 1H), 6.27 (s, 1H), 4.03 (q, 1H), 3.83- |
| | 3.60 (m, 3H), 3.80 (s, 3H), 2.97 (s, 3H). |
| 32 | δ 9.97 (br s, 1H), 8.03-7.93(m, 1H), 6.95-6.84 (m, 1H), 4.21-4.09 (m, 2H), 3.87-3.76 |
| | (m, 1H), 3.83 (s, 3H), 3.45-3.33 (m, 1H), 2.96 (s, 3H). |

### Biological Examples of the Invention

### Test A

### Pre-Emergence Protocol

Seeds of corn (Zea mays, ZEAMX), giant foxtail (Setaria faberi, SETFA), barnyardgrass (Echinochloa crus-galli, ECHCG), ivy-leaved morning glory (Ipomoea hederacea, IPOHE), redroot pigweed (Amaranthus retroflexus, AMARE) and velvetleaf (Abutilon theophrasti, ABUTH) were sown in standard soil in pots. After cultivation for one day under controlled conditions in a glasshouse (at 24/16° C., day/night; 14 h light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 15.625, 62.5 or 250 g/ha of test compound.

The test plants were then grown under controlled conditions in a glasshouse (at 24/16° C., day/night; 14 h light; 65% humidity) and watered twice daily. After 13 d the test was evaluated (100=total damage to plant; 0=no damage to plant). Results are shown below.

### Post-Emergence Protocol

Seeds of corn (Zea mays, ZEAMX), giant foxtail (Setaria faberi, SETFA), barnyardgrass (Echinochloa crus-galli, ECHCG), ivy-leaved morning glory (Ipomoea hederacea, IPOHE)), redroot pigweed (Amaranthus retroflexus, AMARE) and velvetleaf (Abutilon theophrasti, ABUTH) were sown in standard soil in pots. After cultivation for 8 d under controlled conditions in a glasshouse (at 24/16° C., day/night; 14 h light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 15.625, 62.5 or 250 g/ha of test compound.

The test plants were then grown on under controlled conditions in a glasshouse (at 24/16° C., day/night; 14 h light; 65% humidity) and watered twice daily. After 13 d the test was evaluated (100=total damage to plant; 0=no damage to plant). Results are shown below.

TABLE A

| | | | | | | |
|---|---|---|---|---|---|---|
| Cmpd. | | | | | | |
| No. | AMARE | ABUTH | SETFA | ECHCG | ZEAMX | IPOHE |

Pre-Emergence at 250 g/ha

| | | | | | | |
|---|---|---|---|---|---|---|
| 2 | 0 | 0 | 90 | 90 | 10 | 0 |
| 3 | 70 | 10 | 90 | 100 | 90 | 30 |
| 4 | 40 | 0 | 90 | 100 | 80 | 70 |
| 5 | 30 | 10 | 80 | 80 | 0 | 0 |
| 6 | 50 | 70 | 90 | 90 | 60 | 10 |
| 7 | 0 | 0 | 90 | 90 | 90 | 80 |
| 8 | 0 | 0 | 90 | 90 | 70 | 20 |
| 9 | 20 | 0 | 90 | 90 | 20 | 0 |
| 10 | 10 | 0 | 90 | 90 | 70 | 0 |
| 11 | 0 | 0 | 90 | 90 | 20 | 10 |
| 12 | 10 | 0 | 0 | 10 | 20 | 20 |
| 14 | 0 | 0 | 90 | 90 | 70 | 70 |
| 15 | 0 | 0 | 0 | 0 | 0 | 0 |
| 16 | 0 | 0 | 90 | 90 | 30 | 50 |
| 17 | 20 | 10 | 90 | 100 | 90 | 20 |
| 18 | 0 | 0 | 90 | 90 | 80 | 20 |
| 19 | 0 | 0 | 90 | 90 | 20 | 10 |
| 20 | 70 | 0 | 90 | 90 | 20 | 0 |
| 22 | 20 | 0 | 90 | 90 | 40 | 0 |
| 23 | 0 | 0 | 90 | 90 | 40 | 20 |
| 24 | 0 | 0 | 90 | 100 | 80 | 40 |
| 25 | 0 | 40 | 100 | 100 | 20 | 0 |
| 26 | 0 | 0 | 90 | 90 | 90 | 70 |
| 27 | 0 | 0 | 90 | 90 | 10 | 10 |
| 28 | 0 | 0 | 90 | 90 | 0 | 0 |
| 29 | 0 | 0 | 80 | 80 | 10 | 0 |
| 30 | 10 | 0 | 10 | 20 | 0 | 10 |
| 31 | 0 | 0 | 80 | 80 | 20 | 10 |
| 32 | 0 | 0 | 90 | 70 | 10 | 10 |

Post-Emergence at 250 g/ha

| | | | | | | |
|---|---|---|---|---|---|---|
| 2 | 0 | 0 | 80 | 90 | 40 | 0 |
| 3 | 20 | 0 | 90 | 90 | 80 | 0 |
| 4 | 20 | 20 | 90 | 90 | 90 | 20 |
| 5 | 0 | 0 | 80 | 80 | 0 | 0 |
| 6 | 10 | 60 | 70 | 70 | 60 | 10 |
| 7 | 0 | 0 | 80 | 80 | 80 | 0 |
| 8 | 0 | 0 | 80 | 80 | 80 | 30 |
| 9 | 0 | 0 | 90 | 90 | 50 | 0 |
| 10 | 0 | 10 | 90 | 90 | 70 | 30 |
| 11 | 0 | 0 | 80 | 90 | 40 | 0 |
| 12 | 0 | 0 | 0 | 0 | 0 | 0 |
| 14 | 0 | 0 | 90 | 80 | 80 | 50 |
| 15 | 0 | 0 | 0 | 0 | 0 | 0 |
| 16 | 0 | 0 | 70 | 70 | 0 | 0 |
| 17 | 50 | 0 | 80 | 80 | 80 | 0 |
| 18 | 0 | 0 | 70 | 70 | 80 | 50 |
| 19 | 30 | 0 | 80 | 80 | 10 | 0 |
| 20 | 20 | 0 | 80 | 80 | 0 | 0 |
| 22 | 0 | 0 | 80 | 80 | 80 | 30 |
| 23 | 0 | 0 | 80 | 80 | 80 | 30 |
| 24 | 0 | 0 | 80 | 80 | 40 | 50 |
| 25 | 0 | 0 | 80 | 80 | 30 | 40 |
| 26 | 0 | 0 | 90 | 90 | 80 | 50 |
| 27 | 30 | 0 | 70 | 70 | 10 | 20 |
| 28 | 50 | 0 | 70 | 70 | 0 | 0 |
| 29 | 40 | 0 | 60 | 60 | 0 | 10 |
| 30 | 10 | 0 | 50 | 50 | 0 | 0 |
| 31 | 20 | 0 | 80 | 80 | 0 | 40 |
| 32 | 0 | 0 | 80 | 80 | 10 | 10 |

Pre-Emergence at 62.5 g/ha

| | | | | | | |
|---|---|---|---|---|---|---|
| 1 | 0 | 0 | 0 | 0 | 0 | 0 |
| 2 | 0 | 0 | 90 | 90 | 0 | 0 |
| 3 | 20 | 0 | 90 | 100 | 60 | 0 |
| 4 | 20 | 0 | 90 | 90 | 40 | 10 |
| 5 | 0 | 0 | 30 | 20 | 0 | 0 |
| 6 | 20 | 70 | 90 | 90 | 20 | 0 |
| 7 | 0 | 0 | 80 | 80 | 30 | 20 |
| 8 | 0 | 0 | 90 | 90 | 40 | 20 |
| 9 | 0 | 0 | 90 | 90 | 0 | 0 |
| 10 | 0 | 10 | 90 | 90 | 70 | 30 |
| 11 | 0 | 0 | 90 | 90 | 0 | 0 |
| 12 | 0 | 0 | 0 | 0 | 0 | 0 |
| 14 | 0 | 0 | 90 | 80 | 10 | 40 |
| 15 | 0 | 0 | 0 | 0 | 0 | 0 |
| 16 | 0 | 0 | 80 | 80 | 0 | 0 |
| 17 | 10 | 0 | 90 | 100 | 70 | 0 |
| 18 | 0 | 0 | 90 | 90 | 60 | 10 |
| 19 | 0 | 0 | 90 | 90 | 0 | 0 |
| 20 | 60 | 0 | 80 | 80 | 0 | 0 |
| 22 | 10 | 0 | 90 | 90 | 20 | 0 |
| 23 | 0 | 0 | 90 | 90 | 20 | 10 |
| 24 | 0 | 0 | 90 | 100 | 30 | 0 |
| 25 | 0 | 10 | 90 | 100 | 0 | 0 |
| 26 | 0 | 0 | 90 | 90 | 70 | 30 |
| 27 | 0 | 0 | 80 | 90 | 10 | 0 |
| 28 | 0 | 0 | 80 | 70 | 0 | 0 |
| 29 | 0 | 0 | 30 | 70 | 10 | 0 |
| 30 | 0 | 0 | 0 | 10 | 0 | 0 |
| 31 | 0 | 0 | 80 | 80 | 10 | 0 |
| 32 | 0 | 0 | 60 | 50 | 10 | 0 |

Post-Emergence at 62.5 g/ha

| | | | | | | |
|---|---|---|---|---|---|---|
| 1 | 0 | 0 | 0 | 0 | 0 | 0 |
| 2 | 0 | 0 | 70 | 90 | 0 | 0 |
| 3 | 10 | 0 | 90 | 90 | 50 | 0 |
| 4 | 20 | 0 | 90 | 90 | 50 | 0 |
| 5 | 0 | 0 | 50 | 60 | 0 | 0 |
| 6 | 10 | 70 | 70 | 70 | 20 | 20 |
| 7 | 0 | 0 | 70 | 80 | 40 | 20 |
| 8 | 0 | 0 | 70 | 70 | 60 | 0 |
| 9 | 0 | 0 | 90 | 90 | 0 | 0 |
| 10 | 0 | 0 | 80 | 90 | 10 | 0 |
| 11 | 0 | 0 | 80 | 90 | 10 | 0 |
| 12 | 0 | 0 | 0 | 0 | 0 | 0 |
| 14 | 0 | 0 | 80 | 80 | 70 | 30 |
| 15 | 0 | 0 | 0 | 0 | 0 | 0 |
| 16 | 0 | 0 | 60 | 50 | 0 | 0 |
| 17 | 20 | 0 | 70 | 70 | 50 | 0 |
| 18 | 0 | 0 | 70 | 70 | 30 | 20 |
| 19 | 0 | 0 | 70 | 70 | 0 | 0 |
| 20 | 10 | 0 | 70 | 60 | 0 | 0 |
| 22 | 0 | 0 | 70 | 70 | 40 | 10 |
| 23 | 0 | 0 | 80 | 80 | 20 | 10 |
| 24 | 0 | 0 | 70 | 80 | 10 | 20 |
| 25 | 0 | 0 | 70 | 70 | 20 | 40 |
| 26 | 0 | 0 | 80 | 70 | 60 | 20 |
| 27 | 40 | 0 | 50 | 50 | 0 | 10 |
| 28 | 40 | 0 | 30 | 50 | 0 | 0 |
| 29 | 30 | 0 | 20 | 50 | 0 | 0 |
| 30 | 0 | 0 | 20 | 20 | 0 | 0 |
| 31 | 10 | 0 | 80 | 80 | 0 | 30 |
| 32 | 0 | 0 | 60 | 40 | 0 | 0 |

Pre-Emergence at 15.625 g/ha

| | | | | | | |
|---|---|---|---|---|---|---|
| 1 | 0 | 0 | 0 | 0 | 0 | 0 |
| 2 | 10 | 0 | 90 | 90 | 0 | 0 |
| 3 | 20 | 0 | 90 | 100 | 0 | 0 |
| 4 | 0 | 0 | 80 | 90 | 0 | 0 |
| 5 | 0 | 0 | 0 | 0 | 0 | 0 |
| 6 | 20 | 80 | 80 | 80 | 10 | 0 |
| 7 | 0 | 0 | 70 | 70 | 20 | 20 |
| 8 | 0 | 0 | 80 | 80 | 20 | 20 |
| 9 | 0 | 0 | 70 | 80 | 50 | 50 |
| 10 | 0 | 0 | 80 | 50 | 0 | 0 |
| 11 | 0 | 0 | 80 | 20 | 0 | 0 |
| 12 | 0 | 0 | 0 | 0 | 0 | 0 |
| 14 | 0 | 0 | 60 | 60 | 20 | 30 |
| 16 | 0 | 0 | 0 | 0 | 0 | 0 |
| 17 | 0 | 0 | 90 | 90 | 60 | 0 |
| 18 | 0 | 0 | 90 | 90 | 30 | 10 |
| 19 | 0 | 0 | 80 | 70 | 0 | 0 |
| 20 | 50 | 0 | 50 | 50 | 0 | 0 |
| 22 | 0 | 0 | 80 | 70 | 0 | 0 |
| 23 | 0 | 0 | 70 | 70 | 10 | 0 |
| 24 | 0 | 0 | 70 | 90 | 0 | 0 |
| 25 | 0 | 0 | 80 | 90 | 0 | 0 |
| 26 | — | — | — | — | — | — |
| 27 | 0 | 0 | 60 | 70 | 0 | 0 |
| 28 | 0 | 0 | 20 | 30 | 0 | 0 |
| 29 | 0 | 0 | 20 | 30 | 0 | 0 |
| 30 | 0 | 0 | 0 | 0 | 0 | 0 |
| 31 | 0 | 0 | 40 | 60 | 0 | 0 |
| 32 | 0 | 0 | 20 | 30 | 0 | 0 |

Post-Emergence at 15.625 g/ha

| | | | | | | |
|---|---|---|---|---|---|---|
| 1 | 0 | 0 | 0 | 0 | 0 | 0 |
| 2 | 0 | 0 | 0 | 0 | 10 | 0 |
| 3 | 0 | 0 | 80 | 90 | 0 | 0 |
| 4 | 0 | 0 | 90 | 80 | 20 | 0 |
| 5 | 0 | 0 | 10 | 0 | 0 | 0 |
| 6 | 0 | 60 | 0 | 70 | 10 | 40 |
| 7 | 0 | 0 | 60 | 60 | 20 | 20 |
| 8 | 0 | 0 | 70 | 60 | 20 | 20 |
| 9 | 0 | 0 | 40 | 60 | 20 | 10 |
| 10 | 0 | 0 | 50 | 60 | 0 | 0 |
| 11 | 0 | 0 | 60 | 50 | 10 | 0 |
| 12 | 0 | 0 | 0 | 0 | 0 | 0 |
| 14 | 0 | 0 | 80 | 80 | 70 | 10 |
| 15 | 0 | 0 | 0 | 0 | 0 | 0 |
| 16 | 0 | 0 | 0 | 0 | 0 | 0 |
| 17 | 0 | 0 | 60 | 60 | 10 | 0 |
| 18 | 0 | 0 | 50 | 50 | 0 | 10 |
| 19 | 0 | 0 | 70 | 60 | 0 | 0 |
| 20 | 10 | 0 | 30 | 20 | 0 | 0 |
| 22 | 0 | 0 | 50 | 60 | 20 | 0 |
| 23 | 0 | 0 | 60 | 50 | 0 | 0 |
| 24 | 0 | 0 | 20 | 40 | 0 | 10 |
| 25 | 0 | 0 | 30 | 30 | 0 | 10 |
| 26 | — | — | — | — | — | — |
| 27 | 30 | 0 | 10 | 20 | 0 | 10 |
| 28 | 30 | 0 | 10 | 30 | 0 | 0 |
| 29 | 10 | 0 | 10 | 20 | 0 | 0 |
| 30 | 0 | 0 | 10 | 0 | 0 | 0 |
| 31 | 0 | 0 | 40 | 50 | 0 | 20 |
| 32 | 0 | 0 | 20 | 10 | 0 | 0 |

### Test B

Seeds of plant species selected from blackgrass (Alopecurus myosuroides), Italian ryegrass (Lolium multiflorum), wheat, winter (winter wheat, Triticum aestivum), corn (Zea mays), large (Lg) crabgrass (Digitaria sanguinalis), giant foxtail (Setaria faberi), johnsongrass (Sorghum halepense), ragweed (common ragweed, Ambrosia elatior), soybean (Glycine max), barnyardgrass (Echinochloa crus-galli), waterhemp (common waterhemp, Amaranthus rudis), palmer pigweed (Amaranthus palmeri), and surinam grass (Brachiaria decumbens), were planted into a silt loam soil and treated preemergence with test chemicals formulated in a non-phytotoxic solvent mixture which included a surfactant.

Treated plants and controls were maintained in a greenhouse for 19 to 21 days, after which time all species were compared to controls and visually evaluated. Plant response ratings, summarized in Table B, are based on a scale of 0 to 100 where 0 is no effect and 100 is complete control.

TABLE B

| | Compound |
|---|---|
| 125 g ai/ha | 13 |
| Preemergenee | |
| Barnyardgrass | 97 |
| Blackgrass | 18 |
| Corn | 48 |
| Crabgrass, Large | 100 |
| Foxtail, Giant | 98 |
| Johnsongrass | 55 |
| Pigweed, Palmer | 0 |
| Ragweed | 0 |
| Ryegrass, Italian | 92 |
| Soybean | 8 |
| Surinam Grass | 93 |
| Waterhemp | 15 |
| Wheat | 63 |
| 62 g ai/ha | 13 |
| Preemergence | |
| Barnyardgrass | 95 |
| Blackgrass | 13 |
| Corn | 13 |
| Crabgrass, Large | 100 |
| Foxtail, Giant | 98 |
| Johnsongrass | 15 |
| Pigweed, Palmer | 0 |
| Ragweed | 0 |
| Ryegrass, Italian | 65 |
| Soybean | 0 |
| Surinam Grass | 95 |
| Waterhemp | 0 |
| Wheat | 40 |
| 31 g ai/ha | 13 |
| Preemergenee | |
| Barnyardgrass | 95 |
| Blackgrass | 0 |
| Corn | 5 |
| Crabgrass, Large | 100 |
| Foxtail, Giant | 95 |
| Johnsongrass | 8 |
| Pigweed, Palmer | 0 |
| Ragweed | 0 |
| Ryegrass, Italian | 25 |
| Soybean | 0 |
| Surinam Grass | 78 |
| Waterhemp | 0 |
| Wheat | 20 |
| 16 g ai/ha | 13 |
| Preemergence | |
| Barnyardgrass | 85 |
| Blackgrass | 0 |
| Corn | 3 |
| Crabgrass, Large | 100 |
| Foxtail, Giant | 90 |
| Johnsongrass | 0 |
| Pigweed, Palmer | 0 |
| Ragweed | 0 |
| Ryegrass, Italian | 0 |
| Soybean | 0 |
| Surinam Grass | 65 |
| Waterhemp | 0 |
| Wheat | 3 |

### Test C

Seeds of plant species selected from corn (Zea mays), soybean (Glycine max), velvetleaf (Abutilon theophrasti), palmer pigweed (Amaranthus palmeri), waterhemp (common waterhemp, Amaranthus rudis), surinam grass (Brachiaria decumbens), large (Lg) crabgrass (Digitaria sanguinalis), fall panicum (Panicum dichotomiflorum), giant foxtail (Setaria faberi), ragweed (common ragweed, Ambrosia elatior), barnyardgrass (Echinochloa crus-galli), E. Black Nightshade (Solanum ptycanthum), and horseweed (Conyza canadensis), were planted into a silt loam soil and treated preemergence with test chemicals formulated in a non-phytotoxic solvent mixture which included a surfactant.

Treated plants and controls were maintained in a greenhouse for 19 to 21 days, after which time all species were compared to controls and visually evaluated. Plant response ratings, summarized in Table C, are based on a scale of 0 to 100 where 0 is no effect and 100 is complete control.

TABLE C

| | Compound |
|---|---|
| 125 g ai/ha | 13 |
| Preemergenee | |
| Barnyardgrass | 97 |
| Corn | 15 |
| Crabgrass, Large | 98 |
| Foxtail, Giant | 95 |
| Horseweed | 65 |
| Nightshade | 63 |
| Panicum | 48 |
| Pigweed, Palmer | 28 |
| Ragweed | 0 |
| Soybean | 0 |
| Surinam Grass | 99 |
| Velvetleaf | 0 |
| Waterhemp | 20 |
| 62 g ai/ha | 13 |
| Preemergenee | |
| Barnyardgrass | 97 |
| Corn | 0 |
| Crabgrass, Large | 98 |
| Foxtail, Giant | 95 |
| Horseweed | 45 |
| Nightshade | 0 |
| Panicum | 53 |
| Pigweed, Palmer | 0 |
| Ragweed | 0 |
| Soybean | 0 |
| Surinam Grass | 89 |
| Velvetleaf | 0 |
| Waterhemp | 20 |
| 31 g ai/ha | 13 |
| Preemergenee | |
| Barnyardgrass | 68 |
| Corn | 0 |
| Crabgrass, Large | 98 |
| Foxtail, Giant | 88 |
| Horseweed | 30 |
| Nightshade | 0 |
| Panicum | 0 |
| Pigweed, Palmer | 0 |
| Ragweed | 0 |
| Soybean | 0 |
| Surinam Grass | 90 |
| Velvetleaf | 0 |
| Waterhemp | 0 |
| 16 g ai/ha | 13 |
| Preemergenee | |
| Barnyardgrass | 63 |
| Corn | 0 |
| Crabgrass, Large | 98 |
| Foxtail, Giant | 73 |
| Horseweed | 0 |
| Nightshade | 0 |
| Panicum | 0 |
| Pigweed, Palmer | 0 |
| Ragweed | 0 |
| Soybean | 0 |
| Surinam Grass | 58 |
| Velvetleaf | 0 |
| Waterhemp | 0 |
| 8 g ai/ha | 13 |
| Preemergenee | |
| Barnyardgrass | 28 |
| Corn | 0 |
| Crabgrass, Large | 89 |
| Foxtail, Giant | 55 |
| Horseweed | 0 |
| Nightshade | 0 |
| Panicum | 0 |
| Pigweed, Palmer | 0 |
| Ragweed | 0 |
| Soybean | 0 |
| Surinam Grass | 30 |
| Velvetleaf | 0 |
| Waterhemp | 0 |
| 4 g ai/ha | 13 |
| Preemergenee | |
| Barnyardgrass | 0 |
| Corn | 0 |
| Crabgrass, Large | 70 |
| Foxtail, Giant | 0 |
| Horseweed | 0 |
| Nightshade | 0 |
| Panicum | 0 |
| Pigweed, Palmer | 0 |
| Ragweed | 0 |
| Soybean | 0 |
| Surinam Grass | 30 |
| Velvetleaf | 0 |
| Waterhemp | 0 |

### Test D

A formulation containing 50 g/L of the test chemical was prepared by dissolving the active ingredient in a mixture of organic solvents and emulsifier, details of which are provided in the table. This formulation was then mixed with a small, variable amount of acetone to aid dissolution, before addition of a 0.2% v/v aqueous solution of an adjuvant, as the aqueous diluent, to form an aqueous spray solution which contains a predetermined concentration of the active ingredient.

| | CAS Registry | Amount |
|---|---|---|
| Chemical description | number | (% w/w) |
| Castor oil ethoxylate | 61791-12-6 | 10.6 |
| 1-Methyl-2-pyrrolidone | 872-50-4 | 42.2 |
| Dipropylene glycol | 34590-94-8 | 42.2 |
| monomethyl ether | | |

This aqueous spray solution was then sprayed onto plants including maize and wheat that had been seed treated with a herbicide safener, after one day's cultivation (for pre-emergence) or after about 12 days' cultivation (for post-emergence). The plants were grown from seeds sown in standard soil, placed in a glasshouse under controlled conditions (at 24/18° C. or 20/16° C., day/night; 16 h light; 65% humidity). After spray application the plants were then grown in a glasshouse under the same conditions and watered twice daily. After 15 d for post-emergence and 20 DAA for pre-emergence the test was evaluated (100=total damage to plant; 0=no damage to plant).

| EPPO Code | Species | Scientific Name |
|---|---|---|
| TRZAW | Wheat, Winter | Triticum aestivum L. |
| HORVW | Barley, Winter | Hordeum vulgare L. |
| AVEFA | Oats, Wild | Avena fatua L. |
| SINAR | Mustard, Wild | Sinapis arvensis L. |
| BEAVA | Sugarbeet | Beta vulgaris L. |
| ALOMY | Blackgrass | Alopecurus myosuroides HUDS. |
| BRSNN | Rapeseed | Brassica napus L. |
| BROTE | Bromegrass, Downy | Bromus tectorum L. |
| GALAP | Catchweed Bedstraw or Clevers | Galium aparine L. |
| LOLPE | Ryegrass, Perennial | Lolium perenne L. |
| STEME | Chickweed, Common | Stellaria media (L.) VILL./CYR. |
| VERPE | Speedwell, Birdseye | Veronica persica POIR. |
| KCHSC | Kochia | Bassia scoparia |
| CHEAL | Lambsquarters, Common | Chenopodium album L. |
| POAAN | Bluegrass, Annual | Poa annua L. |
| POLCO | Buckwheat, Wild | Polygonum convolvulus L. |
| GLXMA | Soybean | Glycine max (L.) MERR. |
| ZEAMX | Corn | Zea mays L. |
| IPOHE | Morningglory, Ivyleaf | Ipomoea hederacea (L.) JACQ. |
| BIDPI | Beggarticks, Hairy | Bidens pilosa L. |
| CYPES | Nutsedge, Yellow | Cyperus esculentus L. |
| SORVU | Sorghum | Sorghum vulgare PERS. |
| BRAPL | Alexandergrass | Brachiaria plantaginea |
| EPHHL | Poinsettia, Wild | Euphorbia heterophylla L. |
| ORYSA | Rice | Oryza sativa L. |
| ECHCG | Barnyardgrass | Echinochloa crus-galli (L.) P. BEAUV. |
| SETFA | Foxtail, Giant | Setaria faberi HERRM. |
| ABUTH | Velvetleaf | Abutilon theophrasti MEDIK. |
| DIGSA | Crabgrass, Large | Digitaria sanguinalis (L.) SCOP. |
| AMARE | Pigweed, Redroot | Amaranthus retroflexus L. |
| PANMI | Millet | Panicum miliaceum L. |
| SIDSP | Sida, Prickly | Sida spinosa L. |
| ELEIN | Goosegrass | Eleusine indica (L.) GAERTN. |

TABLE D1
“Cereals” Preemergent

| Cmpd. No. | Rates | TRZAW | HORVW | AVEFA | SINAR | BEAVA | ALOMY | BRSNN | BROTE |
|---|---|---|---|---|---|---|---|---|---|
| 2 | 500 | 80 | 20 | 10 | 0 | 10 | 40 | 20 | 20 |
| 2 | 250 | 20 | 10 | 0 | 0 | 0 | 20 | 10 | 20 |
| 2 | 130 | 10 | 0 | 0 | 0 | 0 | 0 | 0 | 10 |
| 2 | 60 | 0 | 0 | 0 | 0 | 0 | 0 | 0 | 0 |
| 2 | 30 | 0 | 0 | 0 | 0 | 0 | 0 | 0 | 0 |
| 2 | 15 | 0 | 0 | 0 | 0 | 0 | 0 | 0 | 0 |
| 3 | 500 | 60 | 20 | 30 | 0 | 0 | 20 | 0 | 0 |
| 3 | 250 | 0 | 0 | 0 | 0 | 0 | 0 | 0 | 0 |
| 3 | 130 | 0 | 0 | 0 | 0 | 0 | 0 | 0 | 0 |
| 3 | 60 | 0 | 0 | 0 | 0 | 0 | 0 | 0 | 0 |
| 3 | 30 | 0 | 0 | 0 | 0 | 0 | 0 | 0 | 0 |
| 3 | 15 | 0 | 0 | 0 | 0 | 0 | 0 | 0 | 0 |
| 4 | 500 | 0 | 20 | 10 | 30 | 0 | 40 | 10 | 10 |
| 4 | 250 | 0 | 0 | 0 | 0 | 0 | 0 | 0 | 0 |
| 4 | 130 | 0 | 0 | 0 | 0 | 0 | 0 | 0 | 0 |
| 4 | 60 | 0 | 0 | 0 | 0 | 0 | 0 | 0 | 0 |
| 4 | 30 | 0 | 0 | 0 | 0 | 0 | 0 | 0 | 0 |
| 4 | 15 | 0 | 0 | 0 | 0 | 0 | 0 | 0 | 0 |
| 6 | 500 | 10 | 20 | 20 | 10 | 0 | 40 | 0 | 0 |
| 6 | 250 | 0 | 10 | 0 | 0 | 0 | 0 | 0 | 0 |
| 6 | 130 | 0 | 0 | 0 | 0 | 0 | 0 | 0 | 0 |
| 6 | 60 | 0 | 0 | 0 | 0 | 0 | 0 | 0 | 0 |
| 6 | 30 | 0 | 0 | 0 | 0 | 0 | 0 | 0 | 0 |
| 6 | 15 | 0 | 0 | 0 | 0 | 0 | 0 | 0 | 0 |
| 7 | 500 | 0 | 0 | 50 | 0 | 0 | 0 | 0 | 0 |
| 7 | 250 | 0 | 0 | 20 | 0 | 0 | 0 | 0 | 0 |
| 7 | 130 | 0 | 0 | 10 | 0 | 0 | 0 | 0 | 0 |
| 7 | 60 | 0 | 0 | 0 | 0 | 0 | 0 | 0 | 0 |
| 7 | 30 | 0 | 0 | 0 | 0 | 0 | 0 | 0 | 0 |
| 7 | 15 | 0 | 0 | 0 | 0 | 0 | 0 | 0 | 0 |
| 8 | 500 | 0 | 10 | 10 | 0 | 0 | 10 | 10 | 0 |
| 8 | 250 | 0 | 0 | 0 | 0 | 0 | 0 | 0 | 0 |
| 8 | 130 | 0 | 0 | 0 | 0 | 0 | 0 | 0 | 0 |
| 8 | 60 | 0 | 0 | 0 | 0 | 0 | 0 | 0 | 0 |
| 8 | 30 | 0 | 0 | 0 | 0 | 0 | 0 | 0 | 0 |
| 8 | 15 | 0 | 0 | 0 | 0 | 0 | 0 | 0 | 0 |
| 9 | 500 | 0 | 0 | 20 | 0 | 0 | 0 | 0 | 0 |
| 9 | 250 | 0 | 0 | 10 | 0 | 0 | 0 | 0 | 0 |
| 9 | 130 | 0 | 0 | 0 | 0 | 0 | 0 | 0 | 0 |
| 9 | 60 | 0 | 0 | 0 | 0 | 0 | 0 | 0 | 0 |
| 9 | 30 | 0 | 0 | 0 | 0 | 0 | 0 | 0 | 0 |
| 9 | 15 | 0 | 0 | 0 | 0 | 0 | 0 | 0 | 0 |
| 14 | 500 | 90 | 90 | 60 | 0 | 10 | 70 | 10 | 50 |
| 14 | 250 | 60 | 60 | 30 | 0 | 0 | 10 | 0 | 10 |
| 14 | 130 | 0 | 10 | 10 | 0 | 0 | 0 | 0 | 0 |
| 14 | 60 | 0 | 0 | 0 | 0 | 0 | 0 | 0 | 0 |
| 14 | 30 | 0 | 0 | 0 | 0 | 0 | 0 | 0 | 0 |
| 14 | 15 | 0 | 0 | 0 | 0 | 0 | 0 | 0 | 0 |
| 17 | 500 | 90 | 90 | 80 | 0 | 10 | 80 | 20 | 60 |
| 17 | 250 | 60 | 40 | 70 | 0 | 0 | 60 | 0 | 40 |
| 17 | 130 | 50 | 10 | 60 | 0 | 0 | 50 | 0 | 0 |
| 17 | 60 | 0 | 0 | 0 | 0 | 0 | 0 | 0 | 0 |
| 17 | 30 | 0 | 0 | 0 | 0 | 0 | 0 | 0 | 0 |
| 17 | 15 | 0 | 0 | 0 | 0 | 0 | 0 | 0 | 0 |
| 18 | 500 | 30 | 40 | 40 | 10 | 0 | 60 | 20 | 40 |
| 18 | 250 | 20 | 0 | 30 | 0 | 0 | 30 | 10 | 30 |
| 18 | 130 | 0 | 0 | 0 | 0 | 0 | 0 | 0 | 10 |
| 18 | 60 | 0 | 0 | 0 | 0 | 0 | 0 | 0 | 0 |
| 18 | 30 | 0 | 0 | 0 | 0 | 0 | 0 | 0 | 0 |
| 18 | 15 | 0 | 0 | 0 | 0 | 0 | 0 | 0 | 0 |
| 19 | 500 | 60 | 20 | 70 | 0 | 0 | 70 | 20 | 30 |
| 19 | 250 | 10 | 0 | 10 | 0 | 0 | 20 | 0 | 10 |
| 19 | 130 | 0 | 0 | 0 | 0 | 0 | 0 | 0 | 0 |
| 19 | 60 | 0 | 0 | 0 | 0 | 0 | 0 | 0 | 0 |
| 19 | 30 | 0 | 0 | 0 | 0 | 0 | 0 | 0 | 0 |
| 19 | 15 | 0 | 0 | 0 | 0 | 0 | 0 | 0 | 0 |
| 22 | 500 | 10 | 0 | 0 | 0 | 0 | 20 | 0 | 0 |
| 22 | 250 | 0 | 0 | 0 | 0 | 0 | 0 | 0 | 0 |
| 22 | 130 | 0 | 0 | 0 | 0 | 0 | 0 | 0 | 0 |
| 22 | 60 | 0 | 0 | 0 | 0 | 0 | 0 | 0 | 0 |
| 22 | 30 | 0 | 0 | 0 | 0 | 0 | 0 | 0 | 0 |
| 22 | 15 | 0 | 0 | 0 | 0 | 0 | 0 | 0 | 0 |

| Cmpd. No. | Rates | GALAP | LOLPE | STEME | VERPE | KCHSC | CHEAL | POAAN | POLCO |
|---|---|---|---|---|---|---|---|---|---|
| 2 | 500 | 0 | 70 | 60 | 0 | 0 | 0 | 90 | 60 |
| 2 | 250 | 0 | 60 | 0 | 0 | 0 | 0 | 50 | 20 |
| 2 | 130 | 0 | 30 | 0 | 0 | 0 | 0 | 10 | 20 |
| 2 | 60 | 0 | 10 | 0 | 0 | 0 | 0 | 0 | 0 |
| 2 | 30 | 0 | 0 | 0 | 0 | 0 | 0 | 0 | 0 |
| 2 | 15 | 0 | 0 | 0 | 0 | 0 | 0 | 0 | 0 |
| 3 | 500 | 0 | 50 | 30 | 60 | 0 | 0 | 60 | 0 |
| 3 | 250 | 0 | 0 | 0 | 0 | 0 | 0 | 50 | 0 |
| 3 | 130 | 0 | 0 | 0 | 0 | 0 | 0 | 40 | 0 |
| 3 | 60 | 0 | 0 | 0 | 0 | 0 | 0 | 20 | 0 |
| 3 | 30 | 0 | 0 | 0 | 0 | 0 | 0 | 20 | 0 |
| 3 | 15 | 0 | 0 | 0 | 0 | 0 | 0 | 0 | 0 |
| 4 | 500 | 0 | 0 | 0 | 0 | 0 | 0 | 90 | 20 |
| 4 | 250 | 0 | 0 | 0 | 0 | 0 | 0 | 80 | 0 |
| 4 | 130 | 0 | 0 | 0 | 0 | 0 | 0 | 50 | 0 |
| 4 | 60 | 0 | 0 | 0 | 0 | 0 | 0 | 40 | 0 |
| 4 | 30 | 0 | 0 | 0 | 0 | 0 | 0 | 0 | 0 |
| 4 | 15 | 0 | 0 | 0 | 0 | 0 | 0 | 0 | 0 |
| 6 | 500 | 20 | 30 | 10 | 20 | 10 | 20 | 70 | 0 |
| 6 | 250 | 10 | 20 | 10 | 10 | 0 | 10 | 20 | 0 |
| 6 | 130 | 0 | 0 | 0 | 0 | 0 | 0 | 10 | 0 |
| 6 | 60 | 0 | 0 | 0 | 0 | 0 | 0 | 0 | 0 |
| 6 | 30 | 0 | 0 | 0 | 0 | 0 | 0 | 0 | 0 |
| 6 | 15 | 0 | 0 | 0 | 0 | 0 | 0 | 0 | 0 |
| 7 | 500 | 0 | 0 | 0 | 0 | 0 | 0 | 0 | 0 |
| 7 | 250 | 0 | 0 | 0 | 0 | 0 | 0 | 0 | 0 |
| 7 | 130 | 0 | 0 | 0 | 0 | 0 | 0 | 0 | 0 |
| 7 | 60 | 0 | 0 | 0 | 0 | 0 | 0 | 0 | 0 |
| 7 | 30 | 0 | 0 | 0 | 0 | 0 | 0 | 0 | 0 |
| 7 | 15 | 0 | 0 | 0 | 0 | 0 | 0 | 0 | 0 |
| 8 | 500 | 0 | 0 | 0 | 0 | 0 | 0 | 60 | 0 |
| 8 | 250 | 0 | 0 | 0 | 0 | 0 | 0 | 0 | 0 |
| 8 | 130 | 0 | 0 | 0 | 0 | 0 | 0 | 0 | 0 |
| 8 | 60 | 0 | 0 | 0 | 0 | 0 | 0 | 0 | 0 |
| 8 | 30 | 0 | 0 | 0 | 0 | 0 | 0 | 0 | 0 |
| 8 | 15 | 0 | 0 | 0 | 0 | 0 | 0 | 0 | 0 |
| 9 | 500 | 10 | 20 | 80 | 0 | 0 | 0 | 50 | 0 |
| 9 | 250 | 0 | 30 | 10 | 0 | 0 | 0 | 10 | 0 |
| 9 | 130 | 0 | 0 | 0 | 0 | 0 | 0 | 10 | 0 |
| 9 | 60 | 0 | 0 | 0 | 0 | 0 | 0 | 0 | 0 |
| 9 | 30 | 0 | 0 | 0 | 0 | 0 | 0 | 0 | 0 |
| 9 | 15 | 0 | 0 | 0 | 0 | 0 | 0 | 0 | 0 |
| 14 | 500 | 0 | 80 | 60 | 20 | 0 | 50 | 80 | 80 |
| 14 | 250 | 0 | 50 | 10 | 0 | 0 | 0 | 80 | 50 |
| 14 | 130 | 0 | 40 | 0 | 0 | 0 | 0 | 20 | 20 |
| 14 | 60 | 0 | 30 | 0 | 0 | 0 | 0 | 0 | 10 |
| 14 | 30 | 0 | 0 | 0 | 0 | 0 | 0 | 0 | 0 |
| 14 | 15 | 0 | 0 | 0 | 0 | 0 | 0 | 0 | 0 |
| 17 | 500 | 20 | 80 | 60 | 90 | 30 | 70 | 90 | 30 |
| 17 | 250 | 0 | 80 | 30 | 60 | 10 | 60 | 80 | 20 |
| 17 | 130 | 0 | 40 | 10 | 30 | 0 | 50 | 70 | 0 |
| 17 | 60 | 0 | 20 | 0 | 0 | 0 | — | 30 | 0 |
| 17 | 30 | 0 | 0 | 0 | 0 | 0 | 0 | 20 | 0 |
| 17 | 15 | 0 | 0 | 0 | 0 | 0 | 0 | 0 | 0 |
| 18 | 500 | 20 | 70 | 10 | 50 | 0 | 30 | 90 | 0 |
| 18 | 250 | 30 | 50 | 20 | 60 | 0 | 20 | 70 | 0 |
| 18 | 130 | 10 | 20 | 10 | 20 | 0 | 20 | 60 | 0 |
| 18 | 60 | 0 | 10 | 0 | 10 | 0 | 0 | 40 | 0 |
| 18 | 30 | 0 | 0 | 0 | 0 | 0 | 0 | 0 | 0 |
| 18 | 15 | 0 | 0 | 0 | 0 | 0 | 0 | 0 | 0 |
| 19 | 500 | 0 | 80 | 60 | 70 | 0 | 20 | 80 | 10 |
| 19 | 250 | 0 | 30 | 10 | 60 | 0 | 10 | 80 | 0 |
| 19 | 130 | 0 | 0 | 0 | 0 | 0 | 0 | 20 | 0 |
| 19 | 60 | 0 | 0 | 0 | 0 | 0 | 0 | 10 | 0 |
| 19 | 30 | 0 | 0 | 0 | 0 | 0 | 0 | 0 | 0 |
| 19 | 15 | 0 | 0 | 0 | 0 | 0 | 0 | 0 | 0 |
| 22 | 500 | 0 | 30 | 40 | 100 | 0 | 40 | 60 | 20 |
| 22 | 250 | 0 | 20 | 30 | 30 | 0 | 80 | 40 | 10 |
| 22 | 130 | 0 | 10 | 10 | 20 | 0 | 10 | 20 | 0 |
| 22 | 60 | 0 | 0 | 10 | 0 | 0 | 20 | 0 | 0 |
| 22 | 30 | 0 | 0 | 0 | 0 | 0 | 0 | 0 | 0 |
| 22 | 15 | 0 | 0 | 0 | 0 | 0 | 0 | 0 | 0 |

| Cmpd. No. | Rates | TRZAW | HORVW | AVEFA | SINAR | BEAVA | ALOMY | BRSNN | BROTE |
|---|---|---|---|---|---|---|---|---|---|
| 24 | 500 | 10 | 20 | 10 | 0 | 0 | 10 | 0 | 0 |
| 24 | 250 | 0 | 0 | 0 | 0 | 0 | 0 | 0 | 0 |
| 24 | 130 | 0 | 0 | 0 | 0 | 0 | 0 | 0 | 0 |
| 24 | 60 | 0 | 0 | 0 | 0 | 0 | 0 | 0 | 0 |
| 24 | 30 | 0 | 0 | 0 | 0 | 0 | 0 | 0 | 0 |
| 24 | 15 | 0 | 0 | 0 | 0 | 0 | 0 | 0 | 0 |
| 25 | 500 | 0 | 0 | 0 | 0 | 0 | 0 | 20 | 10 |
| 25 | 250 | 0 | 0 | 0 | 0 | 0 | 0 | 0 | 0 |
| 25 | 130 | 0 | 0 | 0 | 0 | 0 | 0 | 0 | 0 |
| 25 | 60 | 0 | 0 | 0 | 0 | 0 | 0 | 0 | 0 |
| 25 | 30 | 0 | 0 | 0 | 0 | 0 | 0 | 0 | 0 |
| 25 | 15 | 0 | 0 | 0 | 0 | 0 | 0 | 0 | 0 |
| 26 | 500 | 30 | 30 | 90 | 0 | 0 | 70 | 10 | 10 |
| 26 | 250 | 70 | 40 | 50 | 0 | 0 | 70 | 0 | — |
| 26 | 130 | 10 | 10 | 10 | 0 | 0 | 10 | 0 | 0 |
| 26 | 60 | 0 | 0 | 0 | 0 | 0 | 0 | 0 | 0 |
| 26 | 30 | 0 | 0 | 0 | 0 | 0 | 0 | 0 | 0 |
| 26 | 15 | 0 | 0 | 0 | 0 | 0 | 0 | 0 | 0 |
| 31 | 500 | 0 | 20 | 10 | 20 | 0 | 0 | 0 | 10 |
| 31 | 250 | 0 | 0 | 0 | 0 | 0 | 0 | 0 | 0 |
| 31 | 130 | 0 | 0 | 0 | 0 | 0 | 0 | 0 | 0 |
| 31 | 60 | 0 | 0 | 0 | 0 | 0 | 0 | 0 | 0 |
| 31 | 30 | 0 | 0 | 0 | 0 | 0 | 0 | 0 | 0 |
| 31 | 15 | 0 | 0 | 0 | 0 | 0 | 0 | 0 | 0 |

| Cmpd. No. | Rates | GALAP | LOLPE | STEME | VERPE | KCHSC | CHEAL | POAAN |
|---|---|---|---|---|---|---|---|---|
| 24 | 500 | 0 | 10 | 70 | 60 | 10 | 50 | 40 |
| 24 | 250 | 0 | 0 | 10 | 50 | 0 | 40 | 20 |
| 24 | 130 | 0 | 0 | 0 | 0 | 0 | 0 | 0 |
| 24 | 60 | 0 | 0 | 0 | 0 | 0 | 0 | 0 |
| 24 | 30 | 0 | 0 | 0 | 0 | 0 | 0 | 0 |
| 24 | 15 | 0 | 0 | 0 | 0 | 0 | 0 | 0 |
| 25 | 500 | 0 | 0 | 50 | 10 | 0 | 50 | 0 |
| 25 | 250 | 0 | 0 | 0 | 0 | 0 | 20 | 0 |
| 25 | 130 | 0 | 0 | 0 | 0 | 0 | 0 | 0 |
| 25 | 60 | 0 | 0 | 0 | 0 | 0 | 0 | 0 |
| 25 | 30 | 0 | 0 | 0 | 0 | 0 | 0 | 0 |
| 25 | 15 | 0 | 0 | 0 | 0 | 0 | 0 | 0 |
| 26 | 500 | 20 | 90 | 70 | 80 | 0 | 10 | 100 |
| 26 | 250 | 0 | 90 | 40 | 10 | 0 | 10 | 60 |
| 26 | 130 | 0 | 30 | 10 | 0 | 0 | 0 | 30 |
| 26 | 60 | 0 | 0 | 0 | 0 | 0 | 0 | 0 |
| 26 | 30 | 0 | 0 | 0 | 0 | 0 | 0 | 0 |
| 26 | 15 | 0 | 0 | 0 | 0 | 0 | 0 | 0 |
| 31 | 500 | 0 | 0 | 10 | 50 | 0 | 20 | 10 |
| 31 | 250 | 0 | 0 | 0 | 30 | 0 | 0 | 0 |
| 31 | 130 | 0 | 0 | 0 | 0 | 0 | 0 | 0 |
| 31 | 60 | 0 | 0 | 0 | 0 | 0 | 0 | 0 |
| 31 | 30 | 0 | 0 | 0 | 0 | 0 | 0 | 0 |
| 31 | 15 | 0 | 0 | 0 | 0 | 0 | 0 | 0 |

TABLE D2
“Cereals” Postemergent

| Cmpd. No. | Rates | TRZAW | HORVW | AVEFA | SINAR | BEAVA | ALOMY | BRSNN | BROTE |
|---|---|---|---|---|---|---|---|---|---|
| 2 | 500 | 60 | 30 | 40 | — | 10 | 30 | 40 | 30 |
| 2 | 250 | 20 | 20 | 10 | — | 20 | 10 | 50 | 0 |
| 2 | 130 | 20 | 0 | 0 | — | 10 | 0 | 40 | 0 |
| 3 | 500 | 50 | 30 | 30 | 10 | 10 | 20 | 0 | 0 |
| 3 | 250 | 0 | 0 | 0 | 0 | 10 | 0 | 10 | 0 |
| 3 | 130 | 0 | 0 | 0 | 0 | 0 | 0 | 10 | 0 |
| 6 | 500 | 20 | 30 | 10 | 40 | 0 | 0 | 10 | 10 |
| 6 | 250 | 10 | 10 | 0 | — | 0 | 0 | 0 | 0 |
| 6 | 130 | 0 | 0 | 0 | — | 0 | 0 | 0 | 0 |
| 14 | 500 | 80 | 70 | 80 | — | 20 | 70 | 40 | 30 |
| 14 | 250 | 70 | 70 | 70 | — | 30 | 60 | 50 | 10 |
| 14 | 130 | 10 | 20 | 30 | — | 30 | 40 | 40 | 0 |
| 17 | 500 | 70 | 80 | 60 | 60 | 20 | 70 | 20 | 20 |
| 17 | 250 | 40 | 40 | 30 | — | 20 | 50 | 20 | 10 |
| 17 | 130 | 0 | 10 | 0 | — | 10 | 30 | 0 | 0 |
| 18 | 500 | 40 | 50 | 40 | 40 | 0 | 40 | 20 | 0 |
| 18 | 250 | 10 | 30 | 20 | — | 0 | 30 | 10 | 0 |
| 18 | 130 | 0 | 0 | 0 | — | 0 | 10 | 10 | 0 |
| 19 | 500 | 20 | 40 | 40 | 20 | 10 | 30 | 10 | 0 |
| 19 | 250 | 0 | 0 | 10 | — | 10 | 0 | 20 | 0 |
| 19 | 130 | 0 | 0 | 0 | — | 0 | 0 | 0 | 0 |
| 22 | 500 | 10 | 20 | 10 | 20 | 10 | 10 | 10 | 0 |
| 22 | 250 | 0 | 10 | 0 | — | 0 | 0 | 10 | 0 |
| 22 | 130 | 0 | 0 | 0 | — | 0 | 0 | 10 | 0 |
| 24 | 500 | 0 | 0 | 0 | 0 | 0 | 0 | 0 | 0 |
| 24 | 250 | 0 | 0 | 0 | 0 | 0 | 0 | 0 | 0 |
| 24 | 130 | 0 | 0 | 0 | 0 | 0 | 0 | 0 | 0 |
| 26 | 500 | 40 | 40 | 60 | 10 | 0 | 40 | 0 | 0 |
| 26 | 250 | 0 | 0 | 10 | 0 | 0 | 0 | 0 | 0 |
| 26 | 130 | 0 | 0 | 0 | 0 | 0 | 0 | 0 | 0 |

| Cmpd. No. | Rates | GALAP | LOLPE | STEME | VERPE | KCHSC | CHEAL | POAAN | POLCO |
|---|---|---|---|---|---|---|---|---|---|
| 2 | 500 | 0 | 0 | 0 | 0 | 0 | 20 | 80 | 40 |
| 2 | 250 | 10 | 0 | 0 | 10 | 0 | 0 | 70 | 20 |
| 2 | 130 | 0 | 0 | 0 | 0 | 0 | — | 20 | 0 |
| 3 | 500 | 0 | 0 | 10 | 60 | 0 | 0 | 50 | 0 |
| 3 | 250 | 0 | 0 | 0 | 0 | 0 | 0 | 20 | 0 |
| 3 | 130 | 0 | 0 | 0 | 0 | 0 | 0 | 0 | 0 |
| 6 | 500 | 20 | 0 | 10 | 20 | 10 | 0 | 10 | 0 |
| 6 | 250 | 10 | 0 | 0 | 10 | 0 | 0 | 0 | 0 |
| 6 | 130 | 0 | 0 | 0 | 0 | 0 | 0 | 0 | 0 |
| 14 | 500 | 20 | 50 | 70 | 60 | 20 | 40 | 90 | 40 |
| 14 | 250 | 10 | 20 | 50 | 50 | 20 | — | 90 | 30 |
| 14 | 130 | 0 | 0 | — | 50 | 10 | 50 | 80 | 20 |
| 17 | 500 | 30 | 60 | 30 | 60 | 20 | 50 | 90 | 10 |
| 17 | 250 | 40 | 30 | 0 | 70 | 0 | 60 | 80 | 0 |
| 17 | 130 | 0 | 0 | 0 | 30 | 0 | 0 | 60 | 0 |
| 18 | 500 | 40 | 10 | 20 | 40 | 0 | 20 | 70 | 0 |
| 18 | 250 | 0 | 0 | 0 | 0 | 0 | 0 | 50 | 0 |
| 18 | 130 | 0 | 0 | 0 | 0 | 0 | 0 | 0 | 0 |
| 19 | 500 | 20 | 10 | 0 | 10 | 0 | 40 | 40 | 0 |
| 19 | 250 | 0 | 0 | 0 | 0 | 0 | 0 | 30 | 0 |
| 19 | 130 | 0 | 0 | 0 | 0 | 0 | 0 | 0 | 0 |
| 22 | 500 | 10 | 0 | 30 | 60 | 0 | 30 | 0 | 10 |
| 22 | 250 | 40 | 0 | 0 | 50 | 0 | 20 | 0 | 0 |
| 22 | 130 | 0 | 0 | 0 | 0 | 0 | 0 | 0 | 0 |
| 24 | 500 | 20 | 0 | 0 | 20 | 0 | 40 | 0 | 0 |
| 24 | 250 | 0 | 0 | 0 | 0 | 0 | 10 | 0 | 0 |
| 24 | 130 | 0 | 0 | 0 | 0 | 0 | 0 | 0 | 0 |
| 26 | 500 | 60 | 60 | 0 | 80 | 0 | 10 | 80 | 0 |
| 26 | 250 | 0 | 0 | 0 | 0 | 0 | 0 | 20 | 0 |
| 26 | 130 | 0 | 0 | 0 | 0 | 0 | 0 | 30 | 0 |

TABLE D3
“Maize” Preemergent

| Cmpd. No. | Rates | GLXMA | ZEAMX | IPOHE | BIDPI | CYPES | SORVU | BRAPL | EPHHL |
|---|---|---|---|---|---|---|---|---|---|
| 2 | 500 | 0 | 10 | 0 | 0 | 0 | 0 | 90 | 10 |
| 2 | 250 | 0 | 0 | 0 | 0 | 0 | 0 | 90 | 0 |
| 2 | 130 | 0 | 0 | 0 | 0 | 0 | 0 | 80 | 0 |
| 2 | 60 | 0 | 0 | 0 | 0 | 0 | 0 | 80 | 0 |
| 2 | 30 | 0 | 0 | 0 | 0 | 0 | 0 | 70 | 0 |
| 2 | 15 | 0 | 0 | 0 | 0 | 0 | 0 | 0 | 0 |
| 3 | 500 | 10 | 0 | 0 | 0 | 10 | 0 | 100 | 10 |
| 3 | 250 | 0 | 0 | 0 | 0 | 0 | 0 | 100 | 10 |
| 3 | 130 | 0 | 0 | 0 | 0 | 0 | 0 | 100 | 0 |
| 3 | 60 | 0 | 0 | 0 | 0 | 0 | 0 | 90 | 0 |
| 3 | 30 | 0 | 0 | 0 | 0 | 0 | 0 | 50 | 0 |
| 3 | 15 | 0 | 0 | 0 | 0 | 0 | 0 | 20 | 0 |
| 4 | 500 | 0 | 10 | 10 | 0 | 0 | 50 | 90 | 10 |
| 4 | 250 | 0 | 0 | 0 | 0 | 0 | 40 | 90 | 0 |
| 4 | 130 | 0 | 0 | 0 | 0 | 0 | 10 | 80 | 0 |
| 4 | 60 | 0 | 0 | 0 | 0 | 0 | 10 | 60 | 0 |
| 4 | 30 | 0 | 0 | 0 | 0 | 0 | 0 | 40 | 0 |
| 4 | 15 | 0 | 0 | 0 | 0 | 0 | 0 | 40 | 0 |
| 6 | 500 | 0 | 0 | 0 | 0 | 80 | 0 | 90 | 0 |
| 6 | 250 | 0 | 0 | 0 | 0 | 10 | 0 | 80 | 0 |
| 6 | 130 | 0 | 0 | 0 | 0 | 0 | 0 | 80 | 0 |
| 6 | 60 | 0 | 0 | 0 | 0 | 0 | 0 | 60 | 0 |
| 6 | 30 | 0 | 0 | 0 | 0 | 0 | 0 | 50 | 0 |
| 6 | 15 | 0 | 0 | 0 | 0 | 0 | 0 | 0 | 0 |
| 7 | 500 | 0 | 10 | 0 | 0 | 20 | 80 | 100 | 40 |
| 7 | 250 | 0 | 0 | 0 | 0 | 0 | 70 | 90 | 10 |
| 7 | 130 | 0 | 0 | 0 | 0 | 0 | 20 | 80 | — |
| 7 | 60 | 0 | 0 | 0 | 0 | 0 | 0 | 80 | 0 |
| 7 | 30 | 0 | 0 | 0 | 0 | 0 | 0 | 60 | 0 |
| 7 | 15 | 0 | 0 | 0 | 0 | 0 | 0 | 0 | 0 |
| 8 | 500 | 0 | 10 | 0 | 0 | 0 | 30 | 100 | 70 |
| 8 | 250 | 0 | 0 | 0 | 0 | 0 | 20 | 90 | 0 |
| 8 | 130 | 0 | 0 | 0 | 0 | 0 | 10 | 80 | 0 |
| 8 | 60 | 0 | 0 | 0 | 0 | 0 | — | 70 | 0 |
| 8 | 30 | 0 | 0 | 0 | 0 | 0 | — | 70 | 0 |
| 8 | 15 | 0 | 0 | 0 | 0 | 0 | 0 | 20 | 0 |
| 9 | 500 | 0 | 0 | 0 | 0 | 0 | 0 | 100 | 10 |
| 9 | 250 | 0 | 0 | 0 | 0 | 0 | 0 | 100 | — |
| 9 | 130 | 0 | 0 | 0 | 0 | 0 | 0 | 90 | 0 |
| 9 | 60 | 0 | 0 | 0 | 0 | 0 | 0 | 30 | 0 |
| 9 | 30 | 0 | 0 | 0 | 0 | 0 | 0 | 10 | 0 |
| 9 | 15 | 0 | 0 | 0 | 0 | 0 | 0 | 0 | 0 |
| 14 | 500 | 0 | 50 | 0 | 0 | 0 | 30 | 90 | 20 |
| 14 | 250 | 0 | 20 | 0 | 0 | 0 | 0 | 90 | 10 |
| 14 | 130 | 0 | 10 | 0 | 0 | 0 | 0 | 90 | 0 |
| 14 | 60 | 0 | 0 | 0 | 0 | 0 | 0 | 60 | 0 |
| 14 | 30 | 0 | 0 | 0 | 0 | 0 | 0 | 40 | 0 |
| 14 | 15 | 0 | 0 | 0 | 0 | 0 | 0 | 30 | 0 |
| 17 | 500 | 0 | 30 | 0 | 0 | 0 | 0 | 90 | 10 |
| 17 | 250 | 0 | 10 | 0 | 0 | 0 | 0 | 80 | 0 |
| 17 | 130 | 0 | 0 | 0 | 0 | 0 | 0 | 80 | 0 |
| 17 | 60 | 0 | 0 | 0 | 0 | 0 | 0 | 80 | 0 |
| 17 | 30 | 0 | 0 | 0 | 0 | 0 | 0 | 60 | 0 |
| 17 | 15 | 0 | 0 | 0 | 0 | 0 | 0 | 20 | 0 |
| 18 | 500 | 0 | 0 | 0 | 0 | 0 | 10 | 90 | 20 |
| 18 | 250 | 0 | 0 | 0 | 0 | 0 | 0 | 80 | 10 |
| 18 | 130 | 0 | 0 | 0 | 0 | 0 | 0 | 80 | 0 |
| 18 | 60 | 0 | 0 | 0 | 0 | 0 | 0 | 70 | 0 |
| 18 | 30 | 0 | 0 | 0 | 0 | 0 | 0 | 60 | 0 |
| 18 | 15 | 0 | 0 | 0 | 0 | 0 | 0 | 10 | 0 |
| 19 | 500 | 0 | 0 | 0 | 0 | 0 | 0 | 90 | 0 |
| 19 | 250 | 0 | 0 | 0 | 0 | 0 | 0 | 80 | 0 |
| 19 | 130 | 0 | 0 | 0 | 0 | 0 | 0 | 80 | 0 |
| 19 | 60 | 0 | 0 | 0 | 0 | 0 | 0 | 70 | 0 |
| 19 | 30 | 0 | 0 | 0 | 0 | 0 | 0 | 40 | 0 |
| 19 | 15 | 0 | 0 | 0 | 0 | 0 | 0 | 0 | 0 |
| 22 | 500 | 0 | 0 | 0 | 0 | 0 | 0 | 90 | 0 |
| 22 | 250 | 0 | 0 | 0 | 0 | 0 | 0 | 90 | 0 |
| 22 | 130 | 0 | 0 | 0 | 0 | 0 | 0 | 80 | 0 |
| 22 | 60 | 0 | 0 | 0 | 0 | 0 | 0 | 70 | 0 |
| 22 | 30 | 0 | 0 | 0 | 0 | 0 | 0 | 20 | 0 |
| 22 | 15 | 0 | 0 | 0 | 0 | 0 | 0 | 0 | 0 |

| Cmpd. No. | Rates | ORYSA | ECHCG | SETFA | ABUTH | DIGSA | AMARE | PANMI | SIDSP | ELEIN |
|---|---|---|---|---|---|---|---|---|---|---|
| 2 | 500 | 0 | 90 | 90 | 20 | 90 | 0 | 0 | 0 | 0 |
| 2 | 250 | 0 | 90 | 80 | 0 | 90 | 0 | 0 | 0 | 0 |
| 2 | 130 | 0 | 90 | 80 | 0 | 90 | 0 | 0 | 0 | 0 |
| 2 | 60 | 0 | 80 | 80 | 0 | 90 | 0 | 0 | 0 | 0 |
| 2 | 30 | 0 | 50 | 80 | 0 | 90 | 0 | 0 | 0 | 0 |
| 2 | 15 | 0 | 50 | 0 | 0 | 0 | 0 | 0 | 0 | 0 |
| 3 | 500 | 0 | 100 | 90 | 0 | 100 | — | 0 | 60 | 0 |
| 3 | 250 | 0 | 100 | 90 | 0 | 100 | — | 0 | 60 | 0 |
| 3 | 130 | 0 | 90 | 90 | 0 | 100 | — | 0 | 0 | 0 |
| 3 | 60 | 0 | 90 | 80 | 0 | 90 | — | 0 | — | 0 |
| 3 | 30 | 0 | 90 | 30 | 0 | 90 | — | 0 | 0 | 0 |
| 3 | 15 | 0 | 20 | 0 | 0 | 20 | — | 0 | 0 | 0 |
| 4 | 500 | 0 | 90 | 90 | 0 | 90 | — | 40 | 0 | 10 |
| 4 | 250 | 0 | 90 | 90 | 0 | 90 | — | 20 | — | 10 |
| 4 | 130 | 0 | 90 | 80 | 0 | 90 | — | 0 | — | — |
| 4 | 60 | 0 | 80 | 70 | 0 | 80 | — | 0 | 0 | — |
| 4 | 30 | 0 | 40 | 70 | 0 | 60 | — | 0 | 0 | 0 |
| 4 | 15 | 0 | 0 | 10 | 0 | 0 | — | 0 | 0 | 0 |
| 6 | 500 | 0 | 100 | 90 | 0 | 100 | 0 | 0 | | 0 |
| 6 | 250 | 0 | 90 | 90 | 0 | 90 | 0 | 0 | | 0 |
| 6 | 130 | 0 | 80 | 80 | 0 | 90 | 0 | 0 | | 0 |
| 6 | 60 | 0 | 20 | 10 | 0 | 90 | 0 | 0 | | 0 |
| 6 | 30 | 0 | 20 | 0 | 0 | 30 | 0 | 0 | | 0 |
| 6 | 15 | 0 | 0 | 0 | 0 | 0 | 0 | 0 | | 0 |
| 7 | 500 | 0 | 100 | 100 | 10 | 100 | 0 | 20 | 10 | 70 |
| 7 | 250 | 0 | 100 | 90 | 0 | 100 | 0 | 0 | 0 | 50 |
| 7 | 130 | 0 | 100 | 90 | 0 | 100 | 0 | 0 | 0 | 60 |
| 7 | 60 | 0 | 90 | 80 | 0 | 90 | 0 | 0 | 0 | 10 |
| 7 | 30 | 0 | 90 | 80 | 0 | 80 | 0 | 0 | 0 | 0 |
| 7 | 15 | 0 | 0 | 0 | 0 | 0 | 0 | 0 | 0 | 0 |
| 8 | 500 | 0 | 100 | 100 | 0 | 100 | 0 | 30 | 0 | 0 |
| 8 | 250 | 0 | 90 | 100 | 0 | 100 | 0 | 0 | 0 | 0 |
| 8 | 130 | 0 | 90 | 90 | 0 | 100 | 0 | 0 | 0 | 0 |
| 8 | 60 | 0 | 90 | 70 | 0 | 100 | 0 | 0 | 0 | 0 |
| 8 | 30 | 0 | 90 | 80 | 0 | 90 | 0 | 0 | 0 | 0 |
| 8 | 15 | 0 | 30 | 10 | 0 | 10 | 0 | 0 | 0 | 0 |
| 9 | 500 | 0 | 90 | 90 | 10 | 100 | 0 | 0 | 0 | 0 |
| 9 | 250 | 0 | 90 | 70 | 10 | 90 | — | 0 | 0 | 0 |
| 9 | 130 | 0 | 90 | 50 | 0 | 100 | — | 0 | 0 | 0 |
| 9 | 60 | 0 | 80 | 0 | 0 | 80 | — | 0 | 0 | 0 |
| 9 | 30 | 0 | 20 | 0 | 0 | 80 | 0 | 0 | 0 | 0 |
| 9 | 15 | 0 | 0 | 0 | 0 | 0 | 0 | 0 | 0 | 0 |
| 14 | 500 | 0 | 90 | 90 | 20 | 90 | 0 | 80 | 80 | 0 |
| 14 | 250 | 0 | 90 | 90 | 10 | 90 | 0 | 30 | 80 | 0 |
| 14 | 130 | 0 | 90 | 80 | 0 | 90 | 0 | 0 | 30 | 0 |
| 14 | 60 | 0 | 70 | 80 | 0 | 90 | 0 | 0 | 30 | 0 |
| 14 | 30 | 0 | 50 | 70 | 0 | 80 | 0 | 0 | 10 | 0 |
| 14 | 15 | 0 | 30 | 30 | 0 | 60 | 0 | 0 | 10 | 0 |
| 17 | 500 | 0 | 100 | 90 | 10 | 90 | 0 | 0 | | 0 |
| 17 | 250 | 0 | 100 | 90 | 0 | 90 | 0 | 0 | | 0 |
| 17 | 130 | 0 | 90 | 90 | 0 | 90 | 0 | 0 | | 0 |
| 17 | 60 | 0 | 90 | 90 | 0 | 90 | 0 | 0 | | 0 |
| 17 | 30 | 0 | 90 | 80 | 0 | 90 | 0 | 0 | | 0 |
| 17 | 15 | 0 | 0 | 0 | 0 | 30 | 0 | 0 | | 0 |
| 18 | 500 | 0 | 90 | 90 | 30 | 100 | 0 | 0 | | 0 |
| 18 | 250 | 0 | 90 | 90 | 10 | 90 | 0 | 0 | | 0 |
| 18 | 130 | 0 | 90 | 90 | 0 | 90 | 0 | 0 | | 0 |
| 18 | 60 | 0 | 70 | 80 | 0 | 90 | 0 | 0 | | 0 |
| 18 | 30 | 0 | 30 | 0 | 0 | 80 | 0 | 0 | | 0 |
| 18 | 15 | 0 | 0 | 0 | 0 | 0 | 0 | 0 | | 0 |
| 19 | 500 | 0 | 90 | 90 | 10 | 100 | 0 | 0 | | 0 |
| 19 | 250 | 0 | 90 | 90 | 0 | 90 | 0 | 0 | | 0 |
| 19 | 130 | 0 | 90 | 90 | 0 | 90 | 0 | 0 | | 0 |
| 19 | 60 | 0 | 80 | 80 | 0 | 90 | 0 | 0 | | 0 |
| 19 | 30 | 0 | 20 | 60 | 0 | 90 | 0 | 0 | | 0 |
| 19 | 15 | 0 | 0 | 0 | 0 | 70 | 0 | 0 | | 0 |
| 22 | 500 | 0 | 100 | 90 | 0 | 100 | 0 | 0 | | 0 |
| 22 | 250 | 0 | 90 | 60 | 0 | 100 | 0 | 0 | | 0 |
| 22 | 130 | 0 | 80 | 30 | 0 | 80 | 0 | 0 | | 0 |
| 22 | 60 | 0 | 60 | 0 | 0 | 80 | 0 | 0 | | 0 |
| 22 | 30 | 0 | 0 | 0 | 0 | 20 | 0 | 0 | | 0 |
| 22 | 15 | 0 | 0 | 0 | 0 | 0 | 0 | 0 | | 0 |

| Cmpd. No. | Rates | GLXMA | ZEAMX | IPOHE | BIDPI | CYPES | SORVU | BRAPL | EPHHL |
|---|---|---|---|---|---|---|---|---|---|
| 24 | 500 | 0 | 0 | 0 | 0 | 0 | 0 | 90 | 10 |
| 24 | 250 | 0 | 0 | 0 | 0 | 0 | 0 | 80 | 0 |
| 24 | 130 | 0 | 0 | 0 | 0 | 0 | 0 | 80 | 0 |
| 24 | 60 | 0 | 0 | 0 | 0 | 0 | 0 | 30 | 0 |
| 24 | 30 | 0 | 0 | 0 | 0 | 0 | 0 | 20 | 0 |
| 24 | 15 | 0 | 0 | 0 | 0 | 0 | 0 | 0 | 0 |
| 25 | 500 | 0 | 0 | 0 | 0 | 0 | 0 | 90 | 0 |
| 25 | 250 | 0 | 0 | 0 | 0 | 0 | 0 | 90 | 0 |
| 25 | 130 | 0 | 0 | 0 | 0 | 0 | 0 | 80 | 0 |
| 25 | 60 | 0 | 0 | 0 | 0 | 0 | 0 | 30 | 0 |
| 25 | 30 | 0 | 0 | 0 | 0 | 0 | 0 | 0 | 0 |
| 25 | 15 | 0 | 0 | 0 | 0 | 0 | 0 | 0 | 0 |
| 26 | 500 | 0 | 0 | 0 | 0 | 10 | 0 | 80 | 0 |
| 26 | 250 | 0 | 0 | 0 | 0 | 0 | 0 | 80 | 0 |
| 26 | 130 | 0 | 0 | 0 | 0 | 0 | 0 | 80 | 0 |
| 26 | 60 | 0 | 0 | 0 | 0 | 0 | 0 | 80 | 0 |
| 26 | 30 | 0 | 0 | 0 | 0 | 0 | 0 | 70 | 0 |
| 26 | 15 | 0 | 0 | 0 | 0 | 0 | 0 | 0 | 0 |
| 31 | 500 | 0 | 0 | 0 | 0 | 0 | 0 | 90 | 0 |
| 31 | 250 | 0 | 0 | 0 | 0 | 0 | 0 | 80 | 0 |
| 31 | 130 | 0 | 0 | 0 | 0 | 0 | 0 | 80 | 0 |
| 31 | 60 | 0 | 0 | 0 | 0 | 0 | 0 | 60 | 0 |
| 31 | 30 | 0 | 0 | 0 | 0 | 0 | 0 | 10 | 0 |
| 31 | 15 | 0 | 0 | 0 | 0 | 0 | 0 | 0 | 0 |

| Cmpd. No. | Rates | ORYSA | ECHCG | SETFA | ABUTH | DIGSA | AMARE | PANMI |
|---|---|---|---|---|---|---|---|---|
| 24 | 500 | 0 | 90 | 90 | 20 | 90 | 10 | 0 |
| 24 | 250 | 0 | 90 | 60 | 20 | 90 | 0 | 0 |
| 24 | 130 | 0 | 70 | 30 | 0 | 90 | 0 | 0 |
| 24 | 60 | 0 | 50 | 0 | 0 | 80 | 0 | 0 |
| 24 | 30 | 0 | 10 | 0 | 0 | 30 | 0 | 0 |
| 24 | 15 | 0 | 0 | 0 | 0 | 0 | 0 | 0 |
| 25 | 500 | 0 | 90 | 90 | 0 | 90 | 0 | 0 |
| 25 | 250 | 0 | 90 | 60 | 0 | 90 | 0 | 0 |
| 25 | 130 | 0 | 90 | 60 | 0 | 90 | 0 | 0 |
| 25 | 60 | 0 | 60 | 10 | 0 | 80 | 0 | 0 |
| 25 | 30 | 0 | 20 | 0 | 0 | 60 | 0 | 0 |
| 25 | 15 | 0 | 0 | 0 | 0 | 0 | 0 | 0 |
| 26 | 500 | 0 | 90 | 90 | 0 | 90 | 60 | 0 |
| 26 | 250 | 0 | 60 | 90 | 0 | 90 | 70 | 0 |
| 26 | 130 | 0 | 20 | 90 | 0 | 90 | 0 | 0 |
| 26 | 60 | 0 | 30 | 70 | 0 | 90 | 0 | 0 |
| 26 | 30 | 0 | 10 | 20 | 0 | 90 | 0 | 0 |
| 26 | 15 | 0 | 0 | 0 | 0 | 20 | 0 | 0 |
| 31 | 500 | 0 | 90 | 90 | 0 | 90 | 0 | 0 |
| 31 | 250 | 0 | 80 | 20 | 0 | 90 | 0 | 0 |
| 31 | 130 | 0 | 30 | 30 | 0 | 90 | 0 | 0 |
| 31 | 60 | 0 | 30 | 10 | 0 | 50 | 0 | 0 |
| 31 | 30 | 0 | 0 | 0 | 0 | 10 | 0 | 0 |
| 31 | 15 | 0 | 0 | 0 | 0 | 0 | 0 | 0 |

TABLE D4
“Maize” Postemergent

| Cmpd. No. | Rates | GLXMA | ZEAMX | IPOHE | BIDPI | CYPES | SORVU | BRAPL | EPHHL |
|---|---|---|---|---|---|---|---|---|---|
| 2 | 500 | 10 | 20 | 30 | 10 | 0 | 0 | 80 | 40 |
| 2 | 250 | 0 | 0 | 20 | 0 | 0 | 0 | 80 | 10 |
| 2 | 130 | 0 | 0 | 10 | 0 | 0 | 0 | 70 | 0 |
| 3 | 500 | 20 | 10 | 0 | 0 | 10 | 0 | 70 | 0 |
| 3 | 250 | 10 | 0 | 0 | 0 | 0 | 0 | 60 | 0 |
| 3 | 130 | 10 | 0 | 0 | 0 | 0 | 0 | 40 | 0 |
| 6 | 500 | 0 | 20 | 20 | 0 | 0 | 0 | 70 | 30 |
| 6 | 250 | 0 | 10 | 10 | 0 | 0 | 0 | 60 | 20 |
| 6 | 130 | 0 | 0 | 0 | 0 | 0 | 0 | 50 | 10 |
| 14 | 500 | 0 | 20 | 20 | 30 | 0 | 20 | 80 | 50 |
| 14 | 250 | 0 | 0 | 10 | 20 | 0 | 0 | 70 | 30 |
| 14 | 130 | 0 | 0 | 0 | 10 | 0 | 0 | 80 | 20 |
| 17 | 500 | 10 | 0 | 10 | 0 | 10 | 0 | 80 | 30 |
| 17 | 250 | 0 | 0 | 0 | 0 | 0 | 0 | 80 | 20 |
| 17 | 130 | 0 | 0 | 0 | 0 | 0 | 0 | 70 | 10 |
| 18 | 500 | 0 | 10 | 30 | 20 | 0 | 0 | 80 | 20 |
| 18 | 250 | 0 | 0 | 20 | 0 | 0 | 0 | 70 | 10 |
| 18 | 130 | 0 | 0 | 0 | 0 | 0 | 0 | 60 | 0 |
| 19 | 500 | 20 | 30 | 20 | 10 | 0 | 20 | 80 | 30 |
| 19 | 250 | 0 | 0 | 0 | 0 | 0 | 0 | 70 | 10 |
| 19 | 130 | 0 | 0 | 0 | 0 | 0 | 0 | 60 | 0 |
| 22 | 500 | 40 | 20 | 50 | 20 | 10 | 0 | 70 | 30 |
| 22 | 250 | 10 | 0 | 30 | 0 | 0 | 0 | 50 | 20 |
| 22 | 130 | 0 | 0 | 10 | 0 | 0 | 0 | 0 | 0 |
| 24 | 500 | 10 | 20 | 10 | 0 | 10 | 0 | 50 | 20 |
| 24 | 250 | 0 | 0 | 0 | 0 | 0 | 0 | 40 | 0 |
| 24 | 130 | 0 | 0 | 0 | 0 | 0 | 0 | 0 | 0 |
| 26 | 500 | 0 | 10 | 0 | 0 | 0 | 0 | 80 | 10 |
| 26 | 250 | 0 | 0 | 0 | 0 | 0 | 0 | 60 | 10 |
| 26 | 130 | 0 | 0 | 0 | 0 | 0 | 0 | 50 | 0 |

| Cmpd. No. | Rates | ORYSA | ECHCG | SETFA | ABUTH | DIGSA | AMARE | PANMI | SIDSP | ELEIN |
|---|---|---|---|---|---|---|---|---|---|---|
| 2 | 500 | 10 | 80 | 80 | 0 | 70 | 30 | 0 | 0 | 0 |
| 2 | 250 | 0 | 80 | 70 | — | 70 | 10 | 0 | 0 | 0 |
| 2 | 130 | 0 | — | 70 | — | 60 | 0 | 0 | 0 | 0 |
| 3 | 500 | 20 | 80 | 60 | 0 | 60 | 10 | 0 | 0 | 0 |
| 3 | 250 | 0 | 60 | 0 | 0 | 10 | 0 | 0 | 0 | 0 |
| 3 | 130 | 0 | 40 | 0 | 0 | 0 | 0 | 0 | 0 | 0 |
| 6 | 500 | 0 | 70 | 70 | 20 | 70 | 20 | 0 | — | 0 |
| 6 | 250 | 0 | 50 | 20 | 10 | 60 | — | 0 | — | 0 |
| 6 | 130 | 0 | 0 | 0 | 0 | 0 | 0 | 0 | — | 0 |
| 14 | 500 | 0 | 80 | 70 | 10 | 70 | 30 | 0 | 30 | 0 |
| 14 | 250 | 0 | 80 | 70 | 0 | 70 | 0 | 0 | 0 | 0 |
| 14 | 130 | 0 | — | 70 | 0 | 60 | 0 | 0 | 0 | 0 |
| 17 | 500 | 10 | 80 | 70 | 20 | 80 | 40 | 0 | — | 20 |
| 17 | 250 | 0 | 80 | 70 | 0 | 80 | 20 | 0 | — | 10 |
| 17 | 130 | 0 | 80 | 60 | 0 | 70 | — | 0 | — | 0 |
| 18 | 500 | 0 | 70 | 70 | 30 | 70 | 20 | 10 | — | 0 |
| 18 | 250 | 0 | 60 | 30 | 0 | 60 | 10 | 10 | — | 0 |
| 18 | 130 | 0 | 0 | 0 | 0 | 30 | 0 | 0 | — | 0 |
| 19 | 500 | 10 | 70 | 70 | 20 | 80 | 20 | 40 | — | 60 |
| 19 | 250 | 0 | 20 | 50 | 10 | 70 | 10 | 10 | — | 10 |
| 19 | 130 | 0 | 10 | 0 | 0 | 60 | 0 | 0 | — | 10 |
| 22 | 500 | 0 | 50 | 20 | 30 | 60 | 0 | 40 | — | 40 |
| 22 | 250 | 0 | 40 | 0 | 10 | 60 | 0 | 20 | — | 10 |
| 22 | 130 | 0 | 0 | 0 | 0 | 40 | 0 | 70 | — | 10 |
| 24 | 500 | 10 | 0 | 10 | 10 | 0 | 0 | 10 | — | 0 |
| 24 | 250 | 0 | 0 | 0 | 0 | 0 | 0 | 0 | — | 0 |
| 24 | 130 | 0 | 0 | 0 | 0 | 0 | 0 | 0 | — | 0 |
| 26 | 500 | 0 | 10 | 60 | 0 | 60 | 0 | 0 | — | 10 |
| 26 | 250 | 0 | 0 | 60 | 0 | 10 | 0 | 0 | — | 0 |
| 26 | 130 | 0 | 0 | 0 | 0 | 0 | 0 | 0 | — | 0 |

| Cmpd. No. | Rates | GLXMA | ZEAMX | IPOHE | BIDPI | CYPES | SORVU | BRAPL | EPHHL |
|---|---|---|---|---|---|---|---|---|---|
| 7 | 500 | 0 | 10 | 0 | 0 | 0 | 60 | 80 | 40 |
| 7 | 250 | 0 | 0 | 0 | 0 | 0 | 0 | 80 | 20 |
| 7 | 130 | 0 | 0 | 0 | 0 | 0 | 0 | 0 | 0 |
| 8 | 500 | 0 | 0 | 0 | 0 | 0 | 0 | 70 | 20 |
| 8 | 250 | 0 | 0 | 0 | 0 | 0 | 0 | 70 | 30 |
| 8 | 130 | 0 | 0 | 0 | 0 | 0 | 0 | 10 | — |

| Cmpd. No. | Rates | ORYSA | ECHCG | SETFA | ABUTH | DIGSA | AMARE | PANMI | SIDSP |
|---|---|---|---|---|---|---|---|---|---|
| 7 | 500 | 0 | 70 | 70 | 20 | 80 | 10 | 0 | 30 |
| 7 | 250 | 0 | 70 | 70 | 10 | 80 | 0 | 0 | — |
| 7 | 130 | 0 | 20 | 0 | 0 | 0 | 0 | 0 | — |
| 8 | 500 | 10 | 80 | 80 | 0 | 80 | 0 | 0 | 10 |
| 8 | 250 | 0 | 80 | 60 | 0 | 80 | 0 | 0 | 0 |
| 8 | 130 | 0 | 0 | 0 | 0 | 0 | 0 | 0 | 0 |

What is claimed is:

1. A compound selected from Formula 1, including all stereoisomers, N-oxides, and salts thereof: 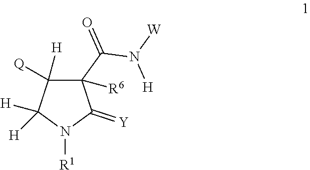
wherein Q is selected from the group consisting of 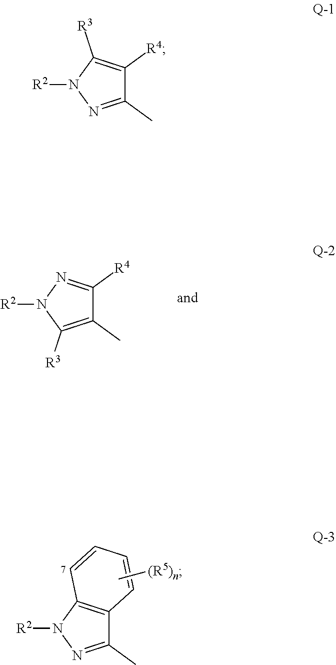
R¹ is H, C₁-C₆ alkyl, C₁-C₆ haloalkyl, C₃-C₇ cycloalkyl or C₄-C₈ cycloalkylalkyl;
R² is C₁-C₆ alkyl or C₁-C₆ haloalkyl;
R³ is halogen, C₁-C₆ alkyl, C₁-C₆ haloalkyl, C₁-C₆ alkoxy or C₁—C haloalkoxy;
Y is O or S;
R⁴ is H, halogen, C₁-C₄ alkyl or C₁-C₄ haloalkyl;
R⁵ is halogen, C₁-C₄ alkyl or C₁-C₄ haloalkyl;
n is 1, 2, 3 or 4;
R⁶ is H, halogen, hydroxy, C₁-C₄ alkoxy, C₁-C₄ haloalkyl or C₁-C₄ alkyl;
W is phenyl or pyridyl, each phenyl or pyridyl optionally substituted with up to 5 R⁹; and
each R⁹ is independently halogen, cyano, nitro, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ nitroalkyl, C₂-C₄ nitroalkenyl, C₂-C₄ alkoxyalkyl, C₂-C₄ haloalkoxyalkyl, C₃-C₄ cycloalkyl, C₃-C₄ halocycloalkyl, cyclopropylmethyl, methylcyclopropyl, C₁-C₄ alkoxy, C₁-C₄ haloalkoxy, C₂-C₄ alkenyloxy, C₂-C₄ haloalkenyloxy, C₃-C₄ alkynyloxy, C₃-C₄ haloalkynyloxy, C₃-C₄ cycloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, hydroxy, formyl, C₂-C₄ alkylcarbonyl, C₂-C₄ alkylcarbonyloxy, C₁-C₄ alkylsulfonyloxy, C₁-C₄ haloalkylsulfonyloxy, amino, C₁-C₄ alkylamino, C₂-C₄ dialkylamino, formylamino, C₂-C₄ alkylcarbonylamino, —SF₅, —SCN, C₃-C₄ trialkylsilyl, trimethylsilylmethyl or trimethylsilylmethoxy;
provided the compound is other than a compound of Formula 1 wherein Q is Q-1; R¹ is H; R² is CH₃; R³ is C(CH₃)₃; R⁴ is H; R⁶ is H; Y is O, W is phenyl substituted with R⁹ at the 2-position; and R⁹ is F.

2. The compound of claim 1 wherein
R¹ is H, C₁-C₆ alkyl, C₁-C₆ haloalkyl or C₄-C₈ cycloalkylalkyl;
R² is C₁-C₃ alkyl or C₁-C₃ haloalkyl;
R³ is halogen, C₁-C₆ alkyl or C₁-C₆ haloalkyl;
Y is O;
R⁴ is H or Cl;
R⁵ is F, Cl or Br;
n is 1, 2 or 3;
R⁶ is H, halogen, hydroxy, C₁-C₄ alkoxy or C₁-C₄ alkyl;
W is phenyl, 3-pyridyl or 4-pyridyl, each phenyl, 3-pyridyl or 4-pyridyl optionally substituted with up to 4 R⁹; and
each R⁹ is independently halogen, cyano, nitro, C₁-C₄ alkyl, C₁-C₄ haloalkyl, C₂-C₄ alkoxyalkyl, C₂-C₄ haloalkoxyalkyl, C₃-C₄ cycloalkyl, C₃-C₄ halocycloalkyl, cyclopropylmethyl, methylcyclopropyl, C₁-C₄ alkoxy, C₁-C₄ haloalkoxy, C₃-C₄ cycloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, hydroxy, formyl, C₂-C₄ alkylcarbonyl, C₂-C₄ alkylcarbonyloxy, C₁-C₄ alkylsulfonyloxy, C₁-C₄ haloalkylsulfonyloxy.

3. The compound of claim 2 wherein
Q is selected from the group consisting of Q-1 and Q-2;
R¹ is H, C₁-C₄ alkyl or C₄-C₅ cycloalkylalkyl;
R² is C₁-C₂ alkyl or C₁-C₂ haloalkyl;
R³ is halogen, C₁-C₃ alkyl or C₁-C₃ haloalkyl;
R⁴ is H;
n is 1 or 2;
R⁶ is H, Cl, hydroxy, OCH₃ or CH₃;
W is phenyl or 3-pyridyl, each phenyl or 3-pyridyl optionally substituted with up to 3 R⁹; and
each R⁹ is independently halogen, cyano, nitro, C₁-C₄ alkyl, C₁-C₄ haloalkyl, C₁-C₄ alkoxy, C₁-C₄ alkylthio, C₁-C₄ alkylsulfinyl or C₁-C₄ alkylsulfonyl.

4. The compound of claim 3 wherein
Q is Q-1;
R¹ is H, CH₃ or cyclopropylmethyl;
R² is CH₃ or CH₂CF₃;
R³ is Cl, CH₃ or CF₃;
R⁶ is H;
W is phenyl substituted with up to 3 R⁹; and
each R⁹ is independently halogen, C₁-C₄ alkyl, C₁-C₄ haloalkyl or C₁-C₄ alkylsulfonyl.

5. The compound of claim 4 wherein
R¹ is H or CH₃;
R² is CH₃;
R³ is CH₃ or CF₃;
R⁶ is H;
each R⁹ is independently F or CF₃; and
R⁹ is at the ortho, meta, or para position of W (relative to the connection to the remainder of Formula 1).

6. The compound of claim 1 that is
(3S,4R)—N-(2,3-difluorophenyl)-1-methyl-4-[1-methyl-5-(trifluoromethyl)-1H-pyrazol-3-yl]-2-oxo-3-pyrrolidinecarboxamide.

7. A herbicidal composition comprising a compound of claim 1 and at least one component selected from the group consisting of surfactants, solid diluents and liquid diluents.

8. A herbicidal composition comprising a compound of claim 1, at least one additional active ingredient selected from the group consisting of other herbicides and herbicide safeners, and at least one component selected from the group consisting of surfactants, solid diluents and liquid diluents.

9. A herbicidal mixture comprising (a) a compound of claim 1, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransfererase (HST) inhibitors, (b14) cellulose biosynthesis inhibitors, (b15) other herbicides including mitotic disruptors, organic arsenicals, asulam, bromobutide, cinmethylin, cumyluron, dazomet, 2-[(2,5-dichlorophenyl)methyl]-4,4-dimethyl-3-isoxazolidinone, difenzoquat, dymron, etobenzanid, flurenol, fosamine, fosamine-ammonium, hydantocidin, metam, methyldymron, oleic acid, oxaziclomefone, pelargonic acid and pyributicarb, (b16) herbicide safeners and salts of compounds of (b1) through (b16).

10. A method for controlling the growth of undesired vegetation comprising contacting the vegetation or its environment with a herbicidally effective amount of a compound of claim 1.
